(19) United States
(12) Patent Application Publication
(10) Pub. No.: US 2025/0302992 A1
(43) Pub. Date: Oct. 2, 2025

(54) Polycistronic Expression of Gut Peptides

(71) Applicant: MeiraGTx UK II Limited, London (GB)

(72) Inventors: Xuecui Guo, Oyster Bay, NY (US); Alexandria Forbes, New York, NY (US)

(21) Appl. No.: 18/719,541

(22) PCT Filed: Dec. 15, 2022
(86) PCT No.: PCT/IB2022/000815

§ 371 Date: Jun. 13, 2024

Related U.S. Application Data
(60) Provisional application No. 63/361,399, filed on Dec. 15, 2021.

(51) Int. Cl. A61K 48/00; A61K 38/00; A61P 3/04; A61P 3/10; C07K 14/605; C12N 15/86

(52) U.S. Cl. CPC A61K 48/005; A61P 3/04; A61P 3/10; C07K 14/605; C12N 15/86; A61K 38/00; C07K 2319/02; C07K 2319/50; C12N 2750/14143; C12N 2800/22; C12N 2840/20

(57) ABSTRACT

Provided herein are monocistronic, bicistronic, and polycistronic expression constructs for the expression of gut peptides and pharmaceutical compositions comprising such expression constructs. Also provided are methods of using such expression constructs, for example, for inducing satiation in a subject in need thereof or for treating obesity in a subject in need thereof.

### CROSS REFERENCE TO RELATED APPLICATIONS

This application is a national stage patent application of International Patent Application No. PCT/IB2022/000815, filed on Dec. 15, 2022, which claims priority to U.S. Provisional Application No. 63/361,399, filed on Dec. 15, 2021; both of which are hereby incorporated by reference in their entireties.

### REFERENCE TO A SEQUENCE LISTING

This application contains a Sequence Listing, which has been submitted electronically in xml format and is hereby incorporated by reference in its entirety. Said xml copy, created on Dec. 19, 2024, is named SeqList2-162027-52301.xml and is 109,978 bytes in size.

### FIELD

The disclosure relates to compositions and methods in the field of molecular biology. Specifically, the disclosure relates to polycistronic expression constructs for the expression of peptides as well as methods of using these polycistronic expression constructs.

### BACKGROUND

Current treatments for obesity involve stimulant medications which are mildly effective and can have detrimental side effects, particularly if used long-term. Other current treatment methods involve invasive bariatric surgery which, while sometimes effective, can involve a variety of serious complications. More recently, satiation gut peptides (also referred to as satiation peptides or gut peptides) have been investigated as potential treatments for obesity.

Satiation gut peptides are chemical messengers that regulate gastrointestinal (GI) functions such as secretion, motility, absorption, digestion, and cell proliferation. These polypeptides are produced by endocrine cells in the stomach, pancreas, or intestine and act locally through autocrine or paracrine mechanisms, or at distant sites in a classical endocrine manner. Penetrating from plasma through the blood-brain barrier, they act by activating specific receptors in the satiety center of the hypothalamus, thus inducing satiation.

Acute supplemental therapy with satiation gut peptides reduces food intake and body weight in obese animal models as well as in lean and obese human subjects.

It is widely acknowledged that satiation gut peptides would not be effective through ingested oral administration since enzymes and acids in the gut would degrade them prior to reaching the blood. Accordingly, novel mechanisms for the expression of satiation gut peptides are urgently needed.

### SUMMARY

Provided herein are expression constructs for the expression of gut peptides and methods of using such expression constructs.

In one aspect, provided is a bicistronic expression construct encoding a polyprotein, wherein:
a. the polyprotein comprises a signal peptide, a first gut peptide, and a second gut peptide; and
b. the polyprotein encoding sequence comprises:
   i. a sequence encoding the signal peptide;
   ii. a sequence encoding the first gut peptide; and
   iii. a sequence encoding the second gut peptide.

In some embodiments, the first gut peptide and/or the second gut peptide comprises a sequence selected from human glucagon like peptide 1 (hGLP-1) peptide, human glucose dependent insulinotropic (hGIP) peptide, human oxyntomodulin (hOXM) peptide, peptide YY (PYY), human glucagon (hGlucagon) peptide, and amylin peptide. In embodiments, the hGLP-1 peptide is the hGLP-1₇₋₃₇ peptide. In embodiments, the hGIP peptide is the hGIP₁₋₄₂ peptide. In some embodiments, the first gut peptide and/or the second gut peptide comprises a sequence that is at least 80% identical to any one of SEQ ID NOS:1-5. In some embodiments, the first gut peptide and/or the second gut peptide comprises a sequence that is at least 90% identical to any one of SEQ ID NOS: 1-5. In some embodiments, the first gut peptide gut peptide and/or the second gut peptide comprises a sequence selected from SEQ ID NOS: 1-5.

In some embodiments, the sequence encoding the first gut peptide and/or the second gut peptide comprises a sequence that is at least 80% identical to any one of SEQ ID NOS:6-12. In some embodiments, the sequence encoding the first gut peptide gut peptide and/or the second gut peptide comprises a sequence that is at least 90% identical to any one of SEQ ID NOS:6-12. In some embodiments, the sequence encoding the first gut peptide gut peptide and/or the second gut peptide comprises a sequence that is selected from SEQ ID NOS:6-12.

In some embodiments, the first gut peptide and the second gut peptide are the same gut peptide. In some embodiments, the sequence encoding the first gut peptide and the sequence encoding the second gut peptide are different. In some embodiments, at least one of the sequence encoding the first gut peptide and the sequence encoding the second gut peptide is codon-optimized. In some embodiments, the sequence encoding the first gut peptide and the sequence encoding the second gut peptide are codon-optimized. In some embodiments, the first gut peptide and the second gut peptide is hGLP-1. In some embodiments, the first gut peptide and the second gut peptide each comprise a sequence that is at least 80% identical to SEQ ID NO:1. In some embodiments, the first gut peptide and the second gut peptide each comprise a sequence that is at least 90% identical to SEQ ID NO:1. In some embodiments, the first gut peptide and the second gut peptide each comprise SEQ ID NO:1.

In some embodiments, the sequence encoding the first gut peptide and the sequence encoding the second gut peptide each comprise a sequence that is at least 80% identical to a sequence selected from SEQ ID NOS:6-8. In some embodiments, the sequence encoding the first gut peptide and the sequence encoding the second gut peptide each comprise a sequence that is at least 90% identical to a sequence selected from SEQ ID NOS:6-8. In some embodiments, the sequences encoding the first and the second gut peptide are selected from SEQ ID NOS:6-8.

In some embodiments, the bicistronic expression construct comprises a sequence encoding a polypeptide that is at least 80% identical to SEQ ID NO:45 or SEQ ID NO:55. In some embodiments, the bicistronic expression construct comprises a sequence encoding a polypeptide that is at least 90% identical to SEQ ID NO:45 or SEQ ID NO:55. In some embodiments, the bicistronic expression construct encodes a polypeptide comprising SEQ ID NO:45 or SEQ ID NO:55.

In some embodiments, the bicistronic expression construct comprises a sequence that is at least 80% identical to SEQ ID NO:50 or SEQ ID NO:57. In some embodiments, the bicistronic expression construct comprises a sequence that is at least 90% identical to SEQ ID NO:50 or SEQ ID NO:57. In some embodiments, the bicistronic expression construct comprises SEQ ID NO:50 or SEQ ID NO:57.

In some embodiments, the first gut peptide and the second gut peptide are different gut peptides. In some embodiments, the first gut peptide and the second gut peptide are selected from the group consisting of hGLP-1 and hGIP. In embodiments, the hGLP-1 peptide is the hGLP-1₇₋₃₇ peptide. In embodiments, the hGIP peptide is the hGIP₁₋₄₂ peptide.

In some embodiments, the bicistronic expression construct encodes a sequence comprising a sequence that is at least 80% identical to any one of SEQ ID NOS:46-49 or SEQ ID NO:56. In some embodiments, the bicistronic expression construct encodes a sequence comprising a sequence that is at least 90% identical to any one of SEQ ID NOS: 46-49 or SEQ ID NO:56. In some embodiments, the bicistronic expression construct encodes a sequence comprising any one of SEQ ID NOS:46-49 or SEQ ID NO:56.

In some embodiments, the bicistronic expression construct comprises a sequence that is at least 80% identical to any one of SEQ ID NOS:51-54 or SEQ ID NO:58. In some embodiments, the bicistronic expression construct comprises a sequence that is at least 90% identical to any one of SEQ ID NOS:51-54 or SEQ ID NO:58. In some embodiments, the bicistronic expression construct comprises a sequence selected from SEQ ID NOS:51-54 or SEQ ID NO:58.

In one aspect, provided is a tricistronic expression construct encoding a polyprotein, wherein:
a. the polyprotein comprises a signal peptide, a first gut peptide, a second gut peptide, and a third gut peptide; and
b. the polyprotein encoding sequence comprises:
   i. a sequence encoding the signal peptide;
   ii. a sequence encoding the first gut peptide;
   iii. a sequence encoding the second gut peptide; and
   iv. a sequence encoding the third gut peptide.

In some embodiments, the first gut peptide, the second gut peptide, and/or the third gut peptide comprises a sequence selected from the group consisting of hGLP-1 peptide, hGIP peptide, hOXM peptide, peptide YY (PYY), hGlucagon peptide, and amylin peptide. In embodiments, the hGLP-1 peptide is the hGLP-1₇₋₃₇ peptide. In embodiments, the hGIP peptide is the hGIP₁₋₄₂ peptide. In some embodiments, the first gut peptide, the second gut peptide, and/or the third gut peptide comprises a sequence that is at least 80% identical to any one of SEQ ID NOS:1-5. In some embodiments, the first gut peptide, the second gut peptide, and/or the third gut peptide comprises a sequence that is at least 90% identical to any one of SEQ ID NOS:1-4. In some embodiments, the first gut peptide, the second gut peptide, and/or the third gut peptide comprises a sequence selected from SEQ ID NOS:1-5.

In some embodiments, the sequence encoding first gut peptide, the second gut peptide, and/or the third gut peptide comprises a sequence that is at least 80% identical to any one of SEQ ID NOS:6-12. In some embodiments, the sequence encoding the first gut peptide, the second gut peptide, and/or the third gut peptide comprises a sequence that is at least 90% identical to any one of SEQ ID NOS:6-12. In some embodiments, the sequence encoding first gut peptide, the second gut peptide, and/or the third gut peptide comprises a sequence that is selected from SEQ ID NOS:6-12.

In some embodiments, the first gut peptide, the second gut peptide, and the third gut peptide are the same gut peptide. In some embodiments, the sequence encoding the first gut peptide, the sequence encoding the second gut peptide, and the sequence encoding the third gut peptide are different. In some embodiments, at least one of the sequence encoding the first gut peptide, the sequence encoding the second gut peptide, and the sequence encoding the third gut peptide is codon-optimized. In some embodiments, the sequence encoding the sequence encoding the first gut peptide, the sequence encoding the second gut peptide, and the sequence encoding the third gut peptide are codon-optimized.

In some embodiments, the first gut peptide, the second gut peptide and the third gut peptide is hGLP-1.

In some embodiments, the tricistronic expression construct encodes a sequence comprising a sequence that is at least 80% identical to any one of SEQ ID NOS:59-61 or SEQ ID NO:75. In some embodiments, the tricistronic expression construct encodes a sequence comprising a sequence that is at least 90% identical to any one of SEQ ID NOS:59-61 or SEQ ID NO:75. In some embodiments, the tricistronic expression construct encodes a sequence comprising a sequence selected from SEQ ID NOS:59-61 or SEQ ID NO:75.

In some embodiments, the tricistronic expression construct comprises a sequence that is at least 80% identical to any one of SEQ ID NOS:67-69 or SEQ ID NO:78. In some embodiments, the tricistronic expression construct comprises a sequence that is at least 90% identical to any one of SEQ ID NOS:67-69 or SEQ ID NO:78. In some embodiments, the tricistronic expression construct comprises a sequence selected from SEQ ID NOS:67-69 or SEQ ID NO:78.

In some embodiments, the first gut peptide, and the second gut peptide are different gut peptides. In some embodiments, the first gut peptide, the second gut peptide, and the third gut peptide are different gut peptides.

In some embodiments, the first gut peptide, the second gut peptide, and the third gut peptide are selected from the group consisting of (1) hGLP-1 peptide, hOXM peptide, and PYY or (2) hGLP-1 peptide, hGlucagon peptide, and hGIP peptide. In embodiments, the hGLP-1 peptide is the hGLP-1₇₋₃₇ peptide. In embodiments, the hGIP peptide is the hGIP₁₋₄₂ peptide.

In some embodiments, the tricistronic expression construct encodes a sequence comprising a sequence that is at least 80% identical to any one of SEQ ID NOS:62-66 or SEQ ID NOS:76-77. In some embodiments, the tricistronic expression construct encodes a sequence comprising a sequence that is at least 90% identical to any one of SEQ ID NOS:62-66 or SEQ ID NOS:76-77. In some embodiments, the tricistronic expression construct encodes a sequence comprising any one of SEQ ID NOS:62-66 or SEQ ID NOS:76-77.

In some embodiments, the tricistronic expression construct comprises a sequence that is at least 80% identical to any one of SEQ ID NOS:70-74 or SEQ ID NOS:79-80. In some embodiments, the tricistronic expression construct comprises a sequence that is at least 90% identical to any one of SEQ ID NOS:70-74 or SEQ ID NOS:79-80. In some embodiments, the tricistronic expression construct comprises any one of SEQ ID NOS:70-74 or SEQ ID NOS:79-80.

In some embodiments, the bicistronic or the tricistronic expression construct encodes a polyprotein, wherein the polyprotein comprises a signal peptide. In some embodiments, the signal peptide is selected from the group consisting of an immunoglobulin M (IgM) signal peptide, human insulin (hInsul) signal peptide, murine Igh protein (mIgh) protein signal peptide, human growth hormone (hGH) signal peptide, murine erythropoietin (mEpo) signal peptide, murine growth hormone-releasing hormone (mGHRH) signal peptide, human albumin signal peptide, and human factor IX (FIX) signal peptide. In some embodiments, the signal peptide comprises a sequence that is at least 80% identical to any one of SEQ ID NOS:13-20. In some embodiments, the signal peptide comprises a sequence that is at least 90% identical to any one of SEQ ID NOS:13-20. In some embodiments, the signal peptide comprises a sequence selected from SEQ ID NOS:13-20. In some embodiments, the sequence encoding the signal peptide comprises a sequence that is at least 80% identical to any one of SEQ ID NOS:21-28. In some embodiments, the sequence encoding the signal peptide comprises a sequence that is at least 90% identical to any one of SEQ ID NOS:21-28. In some embodiments, the sequence encoding the signal peptide comprises a sequence selected from SEQ ID NOS:21-28.

In some embodiments, the bicistronic or the tricistronic expression construct further comprises a promoter sequence. In some embodiments, the promoter is a CMV or a CASI promoter.

In some embodiments, the bicistronic or the tricistronic expression construct encodes a polyprotein comprising a protease cleavage site positioned between the first gut peptide and the second gut peptide. In some embodiments, the tricistronic expression construct encodes a polyprotein wherein the polyprotein further comprises a protease cleavage site that allows release of the first gut peptide, second gut peptide, and/or the third peptide from the polyprotein.

In some embodiments, at least one of the protease cleavage sites is a furin cleavage site.

In some embodiments, the bicistronic expression construct or the tricistronic expression construct comprises a riboswitch comprising an aptamer, wherein the aptamer binds to a small molecule.

In some embodiments, the bicistronic expression construct or the tricistronic expression construct comprises a gene regulation cassette comprising an aptamer, wherein the aptamer binds to a small molecule.

Provided herein is a vector comprising a bicistronic expression or a tricistronic expression construct disclosed herein. In some embodiments, the vector is an adeno-associated virus (AAV) vector.

Provided herein is a cell comprising a vector disclosed herein. In some embodiments, the cell is isolated.

Provided herein is a pharmaceutical composition comprising a vector disclosed herein and a pharmaceutically acceptable excipient.

In one aspect, provided is a method of inducing satiation in a subject in need thereof, the method comprising administering to the subject an expression construct, a vector, or a pharmaceutical composition disclosed herein.

In one aspect, provided is a method of treating obesity in a subject in need thereof, the method comprising administering to the subject an expression construct, a vector, or a pharmaceutical composition disclosed herein.

In one aspect, provided is a method of suppressing appetite in a subject in need thereof, the method comprising administering to the subject an expression construct, a vector, or a pharmaceutical composition disclosed herein.

In one aspect, provided is a method of reducing of reducing weight or reducing weight gain in a subject in need thereof, the method comprising administering to the subject an expression construct, a vector, or a pharmaceutical composition disclosed herein.

In one aspect, provided is a method of improving glucose tolerance in a subject in need thereof, the method comprising administering to the subject an expression construct, a vector, or a pharmaceutical composition disclosed herein.

In one aspect, provided is a method of inducing insulin release in a subject in need thereof, the method comprising administering to the subject an expression construct, a vector, or a pharmaceutical composition disclosed herein.

### BRIEF DESCRIPTION OF THE FIGURES

FIGS. 1A, 1B, and 1C illustrate the expression of gut peptides using monocistronic expression constructs. FIG. 1A. Expression of hGLP-1₇₋₃₇ peptide, a gut peptide, as determined by ELISA. See Tables 5 and 6 for the nomenclature of hGLP-1 expression constructs. FIG. 1B. Expression of hGLP-1₇₋₃₇ peptide, a gut peptide, as determined by ELISA. See Tables 5 and 6 for the nomenclature of hGLP-1 expression constructs. FIG. 1C. Expression of hGIP₁₋₄₂ peptide, a gut peptide, as determined by ELISA. See Tables 5 and 6 for the nomenclature of hGIP expression constructs.

FIGS. 2A, 2B, and 2C illustrate the expression of gut peptides using mono-, bi-, and tricistronic expression constructs. FIG. 2A. Exemplary bi- and tricistronic expression constructs. FIG. 2B. Comparison of monocistronic (GLP-1_M), bicistronic (2xGLP-1_2xB) or tricistronic (3xGLP-1_3xB) expression of the GLP-1₇₋₃₇ peptide as determined by ELISA. The ELISA kit used was designed to detect GLP-17-36. See Tables 5-10 for the nomenclature of expression constructs. FIG. 2C. Comparison of certain monocistronic and tricistronic constructs encoding the GLP1₇₋₃₇ peptide. Expression was determined by ELISA. See Tables 5-6 and 9-10 for the nomenclature of expression constructs.

FIGS. 3A, 3B, 3C, 3D, and 3E illustrate the expression of gut peptides using mono- and tricistronic expression constructs. FIG. 3A. Expression of the GLP-1₇₋₃₇ peptide from bicistronic expression constructs encoding for a polyprotein comprising the GLP-1₇₋₃₇ peptide and the hGIP₁₋₄₂ peptide. See Tables 7 and 8 for the nomenclature of expression constructs. FIG. 3B. Expression of the hGIP₁₋₄₂ peptide from bicistronic expression constructs encoding for a polyprotein comprising the GLP-1₇₋₃₇ peptide and the hGIP₁₋₄₂ peptide. See Tables 7 and 8 for the nomenclature of expression constructs. FIG. 3C. Expression of the GLP-1₇₋₃₇ peptide from monocistronic expression constructs encoding the GLP-1₇₋₃₇ peptide (GLP1_J and GLP-1_L) or the hGIP₁₋₄₂ peptide (GIP_G) and from a tricistronic expression construct expressing the GLP-1₇₋₃₆ peptide, the hGIP₁₋₄₂ peptide, and a hGlucagon peptide (GGG_A). See Tables 5, 6, 9, and 10 for the nomenclature of expression constructs. FIG. 3D. Expression of the hGIP₁₋₄₂ peptide from monocistronic expression constructs encoding the GLP-1₇₋₃₇ peptide (GLP1_J and GLP-1_L) or the hGIP₁₋₄₂ peptide (GIP_G) and from a tricistronic expression construct expressing the GLP-1₇₋₃₇ peptide, the hGIP₁₋₄₂ peptide, and a hGlucagon peptide (GGG_A). See Tables 5, 6, 9, and 10 for the nomenclature of expression constructs. FIG. 3E. Expression of the GLP-1₇₋₃₇ peptide by indicated tricistronic expression constructs (expressing GLP-1₇₋₃₇ peptide, OXM peptide, and PYY). See Tables 9, and 10 for the nomenclature of expression constructs. The ELISA kit used was designed to detect GLP-1₇₋₃₆.

FIGS. 4A, 4B, 4C, 4D, and 4E illustrate the riboswitch-regulated expression of gut peptides. FIG. 4A. Expression of the GLP-1 peptide by the indicated, regulatable bicistronic expression constructs based on GG_L (expressing the hGLP-1₇₋₃₇ peptide and the hGIP₁₋₄₂ peptide) described in Example 4. The concentration of the small molecule inducer is shown in μM. FIG. 4B. Expression of the hGIP₁₋₄₂ peptide by the indicated, regulatable bicistronic expression constructs (expressing the hGLP-1₇₋₃₇ peptide and the hGIP₁₋₄₂ peptide) described in Example 4. No significant expression was observed for 0 mM of the small molecule inducer. The concentration of the small molecule inducer is shown in μM. FIG. 4C. Expression of the hGLP-1₇₋₃₇ peptide by the indicated, regulatable tricistronic expression construct 3xGLP-1_3xC described in Example 4 (comprising three hGLP-1₇₋₃₇ peptide encoding sequences). FIG. 4C. Expression of the hGLP-1₇₋₃₇ peptide by indicated regulatable, bicistronic and tricistronic expression constructs described in Example 4. MX-001 is the small molecule inducer. FIG. 4D. Expression of the hGLP-1₇₋₃₇ peptide by the indicated, regulatable bicistronic expression constructs based on GG_F described in Example 4 (expressing the hGLP-1₇₋₃₆ peptide and the hGIP₁₋₄₂ peptide. FIG. 4E. Expression of PYY from a regulatable tricistronic expression construct expressing a polyprotein comprising GLP-1, hOXM, and PYY expressed PYY. The ELISA kit used was designed to detect GLP-1₇₋₃₆.

FIGS. 5A and 5B illustrate that gut peptides expressed from the polycistronic expression constructs disclosed herein are biologically active. FIG. 5A. Biological activity of the hGLP-1₇₋₃₇ peptide expressed by the indicated mono-, bi-, and tricistronic expression constructs. FIG. 5B. Biological activity of the hGIP₁₋₄₂ peptide expressed by the indicated mono-, bi-, and tricistronic expression constructs. FIG. 5C. Male C57Bl/6 mice were fed with high fat diet (HFD) starting at 6 weeks of age. At week eight, the mice were injected with either PBS or AAV8 vectors containing GG_F. Mice on a low fat diet (LFD) were injected PBS and served as a control group. Animal body weight was monitored before and after AAV injection weekly.

FIGS. 6A and 6B illustrate that GLP-1 and GIP peptides expressed from a AAV8.GG_F_7-GLP-1 vectors improve glucose tolerance in vivo. FIG. 6A. Experimental setup. FIG. 6B. GLP-1 and GIP peptides expressed from a AAV8.GG_F_7-GLP-1 vectors improve glucose tolerance in vivo.

### DETAILED DESCRIPTION OF THE INVENTION

Provided herein are expression constructs encoding for one or more gut peptides as well as methods of using these expression constructs. In embodiments, the gut peptides are expressed as a polyprotein, which is cleaved to produce the desired gut peptides. As used herein, a polyprotein is a protein which is destined for processing to produce two or more polypeptide products.

### Expression Constructs

Provided herein are monocistronic, bicistronic, tricistronic and other polycistronic expression constructs for the expression of gut peptides.

In embodiments, the expression construct is a monocistronic expression construct for the expression of a single polypeptide.

In embodiments, the expression construct is a bicistronic expression construct for the expression of two polypeptides. The two polypeptides may be expressed as a polyprotein and the individual polypeptides may be be released from the polyprotein after proteolytic cleavage.

In embodiments, the expression construct is a tricistronic expression construct for the expression of three polypeptides. The three polypeptides may be expressed as a polyprotein and the individual polypeptides may be be released from the polyprotein after proteolytic cleavage.

In embodiments, the expression construct is a polycistronic expression construct for the expression of two or more polypeptides. The two or more polypeptides may be expressed as a polyprotein and the individual polypeptides may be be released from the polyprotein after proteolytic cleavage. In some embodiments, the polycistronic expression construct expresses two, three, four, five, six, seven, eight, nine, or ten polypeptides. The two or more polypeptides may be the same or different polypeptides.

In one aspect, the expression constructs provided herein encode one or more gut peptides.

### Monocistronic Expression Constructs

In some embodiments, the gut peptide is human glucagon-like peptide 1 (hGLP-1) peptide, human gastric inhibitory peptide (hGIP) peptide, human oxyntomodulin (hOXM) peptide, peptide YY or peptide tyrosine (PYY), human glucagon (hGlucagon) peptide, or amylin peptide (also called insulinoma amyloid polypeptide (IAPP)). In embodiments, the hGLP-1 peptide is the hGLP-1₇₋₃₆ peptide. In embodiments, the hGIP peptide is the hGIP₁₋₄₂ peptide. In some embodiments, the gut peptide is a gut peptide disclosed in Table 1 or a portion of one of the gut peptides disclosed in Table 1.

In some embodiments, provided is an expression construct that encodes for a polypeptide comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:1. In some embodiments, the expression construct encodes for a polypeptide comprising SEQ ID NO:1.

In some embodiments, provided is an expression construct that encodes for a polypeptide comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:2. In some embodiments, the expression construct encodes for a polypeptide comprising SEQ ID NO:2.

In some embodiments, provided is an expression construct that encodes for a polypeptide comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:3. In some embodiments, the expression construct encodes for a polypeptide comprising SEQ ID NO:3.

In some embodiments, provided is an expression construct that encodes for a polypeptide comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:4. In some embodiments, the expression construct encodes for a polypeptide comprising SEQ ID NO:4.

In some embodiments, provided is an expression construct that encodes for a polypeptide comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:5. In some embodiments, the expression construct encodes for a polypeptide comprising SEQ ID NO:5.

TABLE 1
Selected gut peptide amino acid sequences.

| SEQ | | |
|---|---|---|
| ID | Gut | |
| NO | peptide | Sequence |
| 1 | hGLP-1₇₋₃₇ | HAEGTFTSDVSSYLEGQAAKEFIAWLVKGRG |
| 2 | hGIP₁₋₄₂ | YAEGTFISDYSIAMDKIHQQDFVNWLLAQKGKKNDWKHNITQ |
| 3 | hOXM | HSQGTFTSDYSKYLDSRRAQDFVQWLMNTKRNRNNIA |
| 4 | PYY₃₋₃₆ | IKPEAPREDASPEELNRYYASLRHYLNLVTRQRY |
| 5 | hGlucagon | HSQGTFTSDYSKYLDSRRAQDFVQWLMNT |

In some embodiments, the expression construct comprises a sequence disclosed in Table 2 or a portion of a sequence disclosed in Table 2.

In some embodiments, the expression construct comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:6. In some embodiments, the expression construct comprises SEQ ID NO:6.

In some embodiments, the expression construct comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:7. In some embodiments, the expression construct comprises SEQ ID NO:7.

In some embodiments, the expression construct comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:8. In some embodiments, the expression construct comprises SEQ ID NO:8.

In some embodiments, the expression construct comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:9. In some embodiments, the expression construct comprises SEQ ID NO:9.

In some embodiments, the expression construct comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:10. In some embodiments, the expression construct comprises SEQ ID NO:10.

In some embodiments, the expression construct comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:11. In some embodiments, the expression construct comprises SEQ ID NO:11.

In some embodiments, the expression construct comprises a sequence that is at least 8000, at least 85%, at least 90%, at least 91%, at least 92%, at least 9300, at least 94%, at least 9500 at least 9600, at least 9100, at least 9700 at least 9800, or at least 9900 identical to SEQ ID NO: 12. In some embodiments, the expression construct comprises SEQ TD NO: 12.

TABLE 2
Selected gut peptide nucleic acid sequences.

| SEQ | | |
|---|---|---|
| ID | Encoded gut | |
| NO | peptide | Sequence |
| 6 | hGLP-1 | CATGCTGAAGGGACCTTTACCAGTGATGTAAGTTCTTATTT |
| | (encoding sequence | GGAAGGCCAAGCTGCCAAGGAATTCATTGCTTGGCTGGTGA |
| | variant 1) | AAGGCCGAGGA |
| 7 | hGLP-1 variant | CATGCAGAGGGAACATTTACTAGTGATGTCAGTTCATATCT |
| | (encoding sequence | TGAGGGACAAGCTGCTAAAGAATTTATTGCTTGGCTTGTGA |
| | variant 2) | AGGGAAGAGGA |
| 8 | hGLP-1 variant | CATGCTGAAGGGACATTTACCTCAGATGTTTCTTCATACCT |
| | (encoding sequence | GGAAGGACAGGCTGCCAAGGAATTTATTGCATGGCTTGTGA |
| | variant 3) | AAGGCAGGGGC |
| 9 | hGIP | TACGCGGAAGGGACTTTCATCAGTGACTACAGTATTGCCAT |
| | | GGACAAGATTCACCAACAAGACTTTGTGAACTGGCTGCTGG |
| | | CCCAAAAGGGGAAGAAGAATGACTGGAAACACAACATCACC |
| | | CAG |
| 10 | hOXM | CATTCACAGGGCACATTCACCAGTGACTACAGCAAGTATCT |
| | | GGACTCCAGGCGTGCCCAAGATTTTGTGCAGTGGTTGATGA |
| | | ATACCAAGAGGAACAGGAATAACATTGCC |
| 11 | PYY | ATCAAACCCGAGGCTCCCCGCGAAGACGCCTCGCCGGAGGA |
| | | GCTGAACCGCTACTACGCCTCCCTGCGCCACTACCTCAACC |
| | | TGGTCACCCGGCAGCGGTAT |
| 12 | hGlucagon | CATTCACAGGGCACATTCACCAGTGACTACAGCAAGTATCT |
| | | GGACTCCAGGCGTGCCCAAGATTTTGTGCAGTGGTTGATGA |
| | | ATACC |

In some embodiments, the expression construct encodes for a gut peptide, wherein the gut peptide is fused to a signal peptide. In some embodiment, the signal peptide is immunoglobulin M (IgM) signal peptide, human insulin (hInsul) signal peptide, murine Igh protein (mIgh) signal peptide, human growth hormone (hGH) signal peptide, murine erythropoietin (mEpo) signal peptide, murine growth hormone-releasing hormone (mGHRH) signal peptide, human albumin (hAlbumin) signal peptide, or human factor IX (hFIX) signal peptide. In some embodiments, the signal peptide is a signal peptide disclosed in Table 3 or a portion of a signal peptide disclosed in Table 3.

In some embodiments, the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:13. In some embodiments, the signal peptide comprises SEQ ID NO:13.

In some embodiments, the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:14. In some embodiments, the signal peptide comprises SEQ ID NO:14.

In some embodiments, the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:15. In some embodiments, the signal peptide comprises SEQ ID NO:15.

In some embodiments, the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:16. In some embodiments, the signal peptide comprises SEQ ID NO:16.

In some embodiments, the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:17. In some embodiments, the signal peptide comprises SEQ ID NO:17.

In some embodiments, the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:18. In some embodiments, the signal peptide comprises SEQ ID NO:18.

In some embodiments, the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:19. In some embodiments, the signal peptide comprises SEQ ID NO:19.

In some embodiments, the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:20. In some embodiments, the signal peptide comprises SEQ ID NO:20.

TABLE 3
Selected signal peptide amino acid sequences.

| SEQ ID NO | Signal peptide | Sequence |
|---|---|---|
| 13 | IgM | MGWSCIILFLVATATGAHSA |
| 14 | hInsul | MALWMRLLPLLALLALWGPDPAAA |
| 15 | mIgh | MAWVWTLLELMAAAQSIQA |
| 16 | hGH | MATGSRTSLLLAFGLLCLPWLQEGSA |
| 17 | mEpo | MGVPERPTLLLLLSLLLIPLGLPVLC |
| 18 | mGHRH | MLLWVLFVILILTSGSHCS |
| 19 | hAlbumin | MKWVTFISLLFLFSSAYS |
| 20 | hFIX | MQRVNMIMAESPGLITICLLGYLLSAEC |

In some embodiments, the expression construct comprises a sequence encoding a signal peptide, wherein the signal peptide is fused to the gut peptide. In some embodiments, the sequence encoding the signal peptide comprises a sequence disclosed in Table 4 or a portion of a sequence disclosed in Table 4.

In some embodiments, the sequence encoding the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:21. In some embodiments, the sequence encoding the signal peptide comprises SEQ ID NO:21.

In some embodiments, the sequence encoding the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:22. In some embodiments, the sequence encoding the signal peptide comprises SEQ ID NO:22.

In some embodiments, the sequence encoding the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:23. In some embodiments, the sequence encoding the signal peptide comprises SEQ ID NO:23.

In some embodiments, the sequence encoding the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:24. In some embodiments, the sequence encoding the signal peptide comprises SEQ ID NO:24.

In some embodiments, the sequence encoding the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:25. In some embodiments, the sequence encoding the signal peptide comprises SEQ ID NO:25.

In some embodiments, the sequence encoding the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:26. In some embodiments, the sequence encoding the signal peptide comprises SEQ ID NO:26.

In some embodiments, the sequence encoding the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:27. In some embodiments, the sequence encoding the signal peptide comprises SEQ ID NO:27.

In some embodiments, the sequence encoding the signal peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:28. In some embodiments, the sequence encoding the signal peptide comprises SEQ ID NO:28.

TABLE 4
Selected signal peptide nucleic acid sequences.

| SEQ | | |
|---|---|---|
| ID | Encoded signal | |
| NO | peptide | Sequence |
| 21 | IgM | ATGGGATGGAGCTGTATCATCCTCTTCTTGGTAGCAACAGCTA |
| | | CAGGCGCGCACAGTGCA |
| 22 | hInsul | ATGGCCCTGTGGATGCGCCTCCTGCCCCTGCTGGCGCTGCTGG |
| | | CCCTCTGGGGACCTGACCCAGCCGCAGCC |
| 23 | mIgh | ATGGCCTGGGTCTGGACACTCCTGTTTCTGATGGCTGCTGCCC |
| | | AGTCCATTCAGGCC |
| 24 | hGH | ATGGCTACAGGCTCCCGGACGTCCCTGCTCCTGGCTTTTGGCC |
| | | TGCTCTGCCTGCCCTGGCTTCAAGAGGGCAGTGCC |
| 25 | mEpo | ATGGGGGTGCCCGAACGTCCCACCCTGCTGCTTTTACTCTCCT |
| | | TGCTACTGATTCCTCTGGGCCTCCCAGTCCTCTGT |
| 26 | mGHRH | ATGCTGCTCTGGGTGCTCTTTGTGATCCTCATCCTCACCAGTG |
| | | GCTCCCACTGCTCA |
| 27 | hAlbumin | ATGAAGTGGGTAACCTTTATTTCCCTTCTTTTTCTCTTTAGCT |
| | | CGGCTTATTCC |
| 28 | hFIX | ATGCAGCGCGTGAACATGATCATGGCAGAATCACCAGGCCTCA |
| | | TCACCATCTGCCTTTTAGGATATCTACTCAGTGCTGAATGT |

Provided is an expression construct encoding a polypeptide comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to any of the sequences SEQ ID NOS:29-36. Provided is an expression construct encoding a polypeptide comprising any one of SEQ ID NOS:29-36.

Provided is an expression construct encoding a polypeptide comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to any of the sequences SEQ ID NOs:30, 21 or 34. Provided is an expression construct encoding a polypeptide comprising any one of SEQ ID NOs:30, 21 or 34.

Provided is an expression construct encoding a polypeptide comprising any one of the sequences disclosed in Table 5 or a portion of a sequence disclosed in Table 5.

TABLE 5
Amino acid sequences of exemplary monocistronic expression constructs. hGLP-
1 (SEQ ID NO: 1) is shown in bold. hGIP (SEQ ID NO: 2) is underlined. Signal peptide
encoding sequence is shown in italic letters. The position where furin cleavage
occurs is marked with *. Constructs GLP-1_C, N, M and J, respectively, have the
same leader sequence as constructs GIP_C, E, F and G, respectively.

| SEQ | | | Encoded | |
|---|---|---|---|---|
| ID | Construct | Signal | gut | |
| NO | name | peptide | peptide | Sequence |
| 29 | GLP-1_C | IgM SP | hGLP-1 | MGWSCIILFLVATATGAHSAYPYDVPDYAR |
| | | | | KKR*HAEGTFTSDVSSYLEGQAAKEFIAWL |
| | | | | VKGRG |
| 30 | GLP-1_F | hInsul | hGLP-1 | MALWMRLLPLLALLALWGPDPAAAYPYDVP |
| | | | | DYARKKR*HAEGTFTSDVSSYLEGQAAKEF |
| | | | | IAWLVKGRG |
| 31 | GLP-1_I | mIgh | hGLP-1 | MAWVWTLLFLMAAAQSIQAYPYDVPDYARK |
| | | | | KR*HAEGTFTSDVSSYLEGQAAKEFIAWLV |
| | | | | KGRG |
| 32 | GLP-1_J | hGH | hGLP-1 | MATGSRTSLLLAFGLLCLPWLQEGSAFPTI |
| | | | | PLSRLFDNAMLRARKKR*HAEGTFTSDVSS |
| | | | | YLEGQAAKEFIAWLVKGRG |
| 33 | GLP-1_K | mEpo | hGLP-1 | MGVPERPTLLLLLSLLLIPLGLPVLCAPPR |
| | | | | LICDSRVLERYRKKR*HAEGTFTSDVSSYL |
| | | | | EGQAAKEFIAWLVKGRG |
| 34 | GLP-1_L | mGHRH | hGLP-1 | MLLWVLFVILILTSGSHCSLPPSPPERMQR |
| | | | | *HAEGTFTSDVSSYLEGQAAKEFIAWLVKG |
| | | | | RG |
| 35 | GLP-1_M | hAlbumin | hGLP-1 | MKWVTFISLLFLFSSAYSRGVFRR*HAEGT |
| | | | | FTSDVSSYLEGQAAKEFIAWLVKGRG |
| 36 | GLP-1_N | hFIX9 | hGLP-1 | MQRVNMIMAESPGLITICLLGYLLSAECTV |
| | | | | FLDHENANKILNRPKR*HAEGTFTSDVSSY |
| | | | | LEGQAAKEFIAWLVKGRG |
| 111 | GIP_C | IgM SP | hGIP | MGWSCIILFLVATATGAHSAYPYDVPDYAR |
| | | | | KKR*YAEGTFISDYSIAMDKIHQQDFVNWL |
| | | | | LAQKGKKNDWKHNITQ |
| 112 | GIP_E | hFIX9 | hGIP | MQRVNMIMAESPGLITICLLGYLLSAECTV |
| | | | | FLDHENANKILNRPKR*YAEGTFISDYSIA |
| | | | | MDKIHQQDFVNWLLAQKGKKNDWKHNITQ |
| 113 | GIP_F | hAlbumin | hGIP | MKWVTFISLLFLFSSAYSRGVERR*YAEGT |
| | | | | FISDYSIAMDKIHQQDFVNWLLAQKGKKND |
| | | | | WKHNITQ |
| 114 | GIP_G | hGH | hGIP | MATGSRTSLLLAFGLLCLPWLQEGSAFPTI |
| | | | | PLSRLEDNAMLRARKKR*YAEGTFISDYSI |
| | | | | AMDKIHQQDFVNWLLAQKGKKNDWKHNITQ |

Provided is an expression construct comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to any of the sequences SEQ ID NOS:37-44. Provided is an expression construct encoding a polypeptide comprising any one of SEQ TD NOS:37-44.

Provided is an expression construct comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to any of the sequences SEQ ID NOS:38, 39, or 42. Provided is an expression construct encoding a polypeptide comprising any one of SEQ ID NOS: 38, 39, or 42.

Provided is an expression comprising any one of the sequences disclosed in Table 6 or a portion of a sequence disclosed in Table 6.

TABLE 6
Nucleic acid sequences of exemplary monocistronic expression constructs. All
sequences comprise hGLP-1 encoding sequence variant 1 (SEQ ID NO: 6). Signal
peptide encoding sequence is shown in italic upper-case letters. Furin cleavage
site encoding sequence is shown in lower case letters. Gut peptide encoding
sequence is shown in upper case letters (not italic).

| SEQ | | | Encoded | |
|---|---|---|---|---|
| ID | Construct | Signal | gut | |
| NO | name | peptide | peptide | Sequence |
| 37 | GLP-1_C | IgM | hGLP-1 | ATGGGATGGAGCTGTATCATCCTCTTCTTGG |
| | | | | TAGCAACAGCTACAGGCGCGCACAGTGCAta |
| | | | | cccatacgatgttccagattacgctagaaaa |
| | | | | aagagaCATGCTGAAGGGACCTTTACCAGTG |
| | | | | ATGTAAGTTCTTATTTGGAAGGCCAAGCTGC |
| | | | | CAAGGAATTCATTGCTTGGCTGGTGAAAGGC |
| | | | | CGAGGATGA |
| 38 | GLP-1_F | hInsul | hGLP-1 | ATGGCCCTGTGGATGCGCCTCCTGCCCCTGC |
| | | | | TGGCGCTGCTGGCCCTCTGGGGACCTGACCC |
| | | | | AGCCGCAGCCtacccatacgatgttccagat |
| | | | | tacgctagaaaaaagagaCATGCTGAAGGGA |
| | | | | CCTTTACCAGTGATGTAAGTTCTTATTTGGA |
| | | | | AGGCCAAGCTGCCAAGGAATTCATTGCTTGG |
| | | | | CTGGTGAAAGGCCGAGGATGA |
| 39 | GLP-1_I | mIgh | hGLP-1 | ATGGCCTGGGTCTGGACACTCCTGTTTCTGA |
| | | | | TGGCTGCTGCCCAGTCCATTCAGGCCtaccc |
| | | | | atacgatgttccagattacgctagaaaaaag |
| | | | | agaCATGCTGAAGGGACCTTTACCAGTGATG |
| | | | | TAAGTTCTTATTTGGAAGGCCAAGCTGCCAA |
| | | | | GGAATTCATTGCTTGGCTGGTGAAAGGCCGA |
| | | | | GGATGA |
| 40 | GLP-1_J | hGH | hGLP-1 | ATGGCTACAGGCTCCCGGACGTCCCTGCTCC |
| | | | | TGGCTTTTGGCCTGCTCTGCCTGCCCTGGCT |
| | | | | TCAAGAGGGCAGTGCCttcccaaccattccc |
| | | | | ttatccaggctttttgacaacgctatgctcc |
| | | | | gcgccagaaaaaagagaCATGCTGAAGGGAC |
| | | | | CTTTACCAGTGATGTAAGTTCTTATTTGGAA |
| | | | | GGCCAAGCTGCCAAGGAATTCATTGCTTGGC |
| | | | | TGGTGAAAGGCCGAGGATGA |
| 41 | GLP-1_K | mEpo | hGLP-1 | ATGGGGGTGCCCGAACGTCCCACCCTGCTGC |
| | | | | TTTTACTCTCCTTGCTACTGATTCCTCTGGG |
| | | | | CCTCCCAGTCCTCTGTgctcccccacgcctc |
| | | | | atctgcgacagtcgagttctggagaggtaca |
| | | | | gaaaaaagagaCATGCTGAAGGGACCTTTAC |
| | | | | CAGTGATGTAAGTTCTTATTTGGAAGGCCAA |
| | | | | GCTGCCAAGGAATTCATTGCTTGGCTGGTGA |
| | | | | AAGGCCGAGGATGA |
| 42 | GLP-1_L | mGHRH | hGLP-1 | ATGCTGCTCTGGGTGCTCTTTGTGATCCTCA |
| | | | | TCCTCACCAGTGGCTCCCACTGCTCActgcc |
| | | | | cccctcacctcccttcaggatgcagcgaCAT |
| | | | | GCTGAAGGGACCTTTACCAGTGATGTAAGTT |
| | | | | CTTATTTGGAAGGCCAAGCTGCCAAGGAATT |
| | | | | CATTGCTTGGCTGGTGAAAGGCCGAGGATGA |
| 43 | GLP-1_M | hAlbumin | hGLP-1 | ATGAAGTGGGTAACCTTTATTTCCCTTCTTT |
| | | | | TTCTCTTTAGCTCGGCTTATTCCaggggtgt |
| | | | | gtttcgtcgaCATGCTGAAGGGACCTTTACC |
| | | | | AGTGATGTAAGTTCTTATTTGGAAGGCCAAG |
| | | | | CTGCCAAGGAATTCATTGCTTGGCTGGTGAA |
| | | | | AGGCCGAGGATGA |
| 44 | GLP-1_N | hFIX9 | hGLP-1 | ATGCAGCGCGTGAACATGATCATGGCAGAAT |
| | | | | CACCAGGCCTCATCACCATCTGCCTTTTAGG |
| | | | | ATATCTACTCAGTGCTGAATGTacagttttt |
| | | | | cttgatcatgaaaacgccaacaaaattctga |
| | | | | atcggccaaagaggCATGCTGAAGGGACCTT |
| | | | | TACCAGTGATGTAAGTTCTTATTTGGAAGGC |
| | | | | CAAGCTGCCAAGGAATTCATTGCTTGGCTGG |
| | | | | TGAAAGGCCGAGGATGA |

### Bicistronic Expression Constructs

In one aspect, provided is a bicistronic expression construct encoding a polyprotein, wherein:
a. the polyprotein comprises a signal peptide, a first gut peptide, and a second gut peptide; and
b. the polyprotein encoding sequence comprises:
   i. a sequence encoding the signal peptide;
   ii. a sequence encoding the first gut peptide; and
   iii. a sequence encoding the second gut peptide.

In some embodiments, the first gut peptide and/or the second gut peptide comprises a sequence selected from the group consisting of hGLP-1 peptide, hGIP peptide, hOXM peptide, PYY, hGlucagon peptide, and amylin peptide. In embodiments, the hGLP-1 peptide is the hGLP-1₇₋₃₇ peptide. In embodiments, the hGIP peptide is the hGIP₁₋₄₂ peptide. In some embodiments, the first gut peptide and/or the second gut peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOS:1-5. In some embodiments, the first gut peptide and/or the second gut peptide comprises a sequence that is at least 90% identical to any one of SEQ ID NOS: 1-5. In some embodiments, the first gut peptide gut peptide and/or the second gut peptide comprises a sequence selected from SEQ ID NOS: 1-5.

In some embodiments, the sequence encoding the first gut peptide gut peptide and/or the second gut peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOS:6-12. In some embodiments, the sequence encoding the first gut peptide gut peptide and/or the second gut peptide comprises a sequence that is at least 90% identical to any one of SEQ ID NOS:6-12. In some embodiments, the sequence encoding the first gut peptide gut peptide and/or the second gut peptide comprises a sequence that is selected from SEQ ID NOS:6-12.

In some embodiments, the first gut peptide and the second gut peptide are the same gut peptide. In some embodiments, the first gut peptide and the second gut peptide are the same gut peptide, but the sequence encoding the first gut peptide and the sequence encoding the second gut peptide are different. In some embodiments, at least one of the sequence encoding the first gut peptide and the sequence encoding the second gut peptide is codon-optimized. In some embodiments, the sequence encoding the first gut peptide and the sequence encoding the second gut peptide are codon-optimized. In some embodiments, the first gut peptide and the second gut peptide is hGLP-1.

In some embodiments, the first gut peptide and the second gut peptide comprise a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:1. In some embodiments, the first gut peptide and the second gut peptide comprise a sequence that is at least 90% identical to SEQ ID NO:1. In some embodiments, the first gut peptide and the second gut peptide comprise SEQ ID NO:1.

In some embodiments, the sequence encoding the first gut peptide and the sequence encoding the second gut peptide comprise a sequence that is at least 80%, at least 85%, at least 90%, at least 91%, at least 92%, at least 93%, at least 94%, at least 95%, at least 96%, at least 91%, at least 97%, at least 98%, or at least 99% identical to a sequence selected from SEQ ID NOS:6-8. In some embodiments, the sequence encoding the first gut peptide and the sequence encoding the second gut peptide comprise a sequence that is at least 90% identical to a sequence selected from SEQ ID NOS:6-8. In some embodiments, the sequences encoding the first and the second gut peptide are selected from SEQ ID NOS:6-8.

In some embodiments, the bicistronic expression construct comprises a sequence disclosed in Table 7 or a portion of a sequence disclosed in Table 7.

In some embodiments, the bicistronic expression construct comprises a sequence encoding a polypeptide that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:45 or SEQ ID NO:55. In some embodiments, the bicistronic expression construct comprises a sequence encoding a polypeptide that is at least 90% identical to SEQ ID NO:45 or SEQ ID NO:55 In some embodiments, the bicistronic expression construct encodes a polypeptide comprising SEQ ID NO:45 or SEQ ID NO:55.

In some embodiments, the bicistronic expression construct encodes a sequence comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOS:46-49 or SEQ ID NO:56. In some embodiments, the bicistronic expression construct encodes a sequence comprising a sequence that is at least 90% identical to any one of SEQ ID NOS:46-49 or SEQ ID NO:56. In some embodiments, the bicistronic expression construct encodes a sequence comprising any one of SEQ ID NOS:46-49 or SEQ ID NO:56.

TABLE 7
Amino acid sequences of exemplary bicistronic expression constructs. hGLP-1
(SEQ ID NO: 1) is shown in bold. hGIP (SEQ ID NO: 2) is underlined. The position
where furin cleavage occurs is marked with

| SEQ | | | | |
|---|---|---|---|---|
| ID | Construct | Signal | Gut | |
| NO | name | peptide | peptides | Sequence |
| 45 | 2xGLP- | hAlbumin | hGLP-1, | MKWVTFISLLFLFSSAYSRGVFRR*HAEGT |
| | 1_2xB | | hGLP-1 | FTSDVSSYLEGQAAKEFIAWLVKGRGRKKR |
| | | | | *HAEGTFTSDVSSYLEGQAAKEFIAWLVKG |
| | | | | RG |
| 46 | GG_J | hGH | hGLP-1, | MATGSRTSLLLAFGLLCLPWLQEGSAFPTI |
| | | | | PLSRLEDNAMLRARKKR*HAEGTFTSDVSS |
| | | | | YLEGQAAKEFIAWLVKGRGRKKR*YAEGTF |
| | | | | ISDYSIAMDKIHQQDFVNWLLAQKGKKNDW |
| | | | hGIP | KHNITQ |
| 47 | GG_F | hInsul | hGLP-1, | MALWMRLLPLLALLALWGPDPAAAYPYDVP |
| | | | hGIP | DYARKKR*HAEGTFTSDVSSYLEGQAAKEF |
| | | | | IAWLVKGRGRKKR*YAEGTFISDYSIAMDK |
| | | | | IHQQDFVNWLLAQKGKKNDWKHNITQ |
| 48 | GG_L | mGHRH | hGLP-1, | MLLWVLFVILILTSGSHCSLPPSPPERMQR |
| | | | hGIP | *HAEGTFTSDVSSYLEGQAAKEFIAWLVKG |
| | | | | RGRKKR*YAEGTFISDYSIAMDKIHQQDFV |
| | | | | NWLLAQKGKKNDWKHNITQ |
| 49 | GG_M | hAlbumin | hGLP-1, | MKWVTFISLLFLFSSAYSRGVFRR*HAEGT |
| | | | hGIP | FTSDVSSYLEGQAAKEFIAWLVKGRGRKKR |
| | | | | *YAEGTFISDYSIAMDKIHQQDFVNWLLAQ |
| | | | | KGKKNDWKHNITQ |
| 55 | 2xGLP-1 | n/a | hGLP-1, | HAEGTFTSDVSSYLEGQAAKEFIAWLVKGR |
| | without | | hGLP-1 | GRKKR*HAEGTFTSDVSSYLEGQAAKEFIA |
| | signal | | | WLVKGRG |
| | peptide | | | |
| 56 | GG | n/a | hGLP-1, | HAEGTFTSDVSSYLEGQAAKEFIAWLVKGR |
| | without | | hGIP | GRKKR*YAEGTFISDYSIAMDKIHQQDFVN |
| | signal | | | WLLAQKGKKNDWKHNITQ |
| | peptide | | | |

In some embodiments, the bicistronic expression comprises a sequence disclosed in Table 8 or a portion of a sequence disclosed in Table 8.

In some embodiments, the bicistronic expression construct comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to SEQ ID NO:50 or SEQ ID NO:57. In some embodiments, the bicistronic expression construct comprises a sequence that is at least 90% identical to SEQ ID NO:50 or SEQ TD NO:57 In some embodiments, the bicistronic expression construct comprises SEQ ID NO:50 or SEQ ID NO:57.

In some embodiments, the bicistronic expression construct comprises a sequence that is at least 80%, at least 85%, at least 90%, at least 91%, at least 92%, at least 93%, at least 94%, at least 95%, at least 96%, at least 91%, at least 97%, at least 98%, or at least 99 identical to any one of SEQ ID NOS:51-54 or SEQ ID NO:58. In some embodiments, the bicistronic expression construct comprises a sequence that is at least 90% identical to any one of SEQ ID NOS:51-54 or SEQ ID NO:58. In some embodiments, the bicistronic expression construct comprises a sequence selected from SEQ ID NOS:51-54 or SEQ ID NO:58.

TABLE 8
Nucleic acid sequences of exemplary bicistronic expression constructs. All
sequences comprise hGLP-1 encoding sequence variant 1 (SEQ ID NO: 6). Signal peptide
encoding sequence is shown in italic upper-case letters. Furin clevage site
encoding sequence is shown in lower case letters. Gut peptide encoding sequence
is shown in upper case letters (not italic).

| SEQ | | | | |
|---|---|---|---|---|
| ID | Construct | Signal | Gut | |
| NO | name | peptide | peptides | Sequence |
| 50 | 2xGLP- | hAlbumin | hGLP- | ATGAAGTGGGTAACCTTTATTTCCCTTCTTT |
| | 1_2xB | | 1, | TTCTCTTTAGCTCGGCTTATTCCaggggtgt |
| | | | hGLP-1 | gtttcgtcgaCATGCTGAAGGGACCTTTACC |
| | | | | AGTGATGTAAGTTCTTATTTGGAAGGCCAAG |
| | | | | CTGCCAAGGAATTCATTGCTTGGCTGGTGAA |
| | | | | AGGCCGAGGAagaaaaaagagaCATGCAGAG |
| | | | | GGAACATTTACTAGTGATGTCAGTTCATATC |
| | | | | TTGAGGGACAAGCTGCTAAAGAATTTATTGC |
| | | | | TTGGCTTGTGAAGGGAAGAGGAtga |
| 51 | GG_J | hGH | hGLP- | ATGGCTACAGGCTCCCGGACGTCCCTGCTCC |
| | | | 1, hGIP | TGGCTTTTGGCCTGCTCTGCCTGCCCTGGCT |
| | | | | TCAAGAGGGCAGTGCCttcccaaccattccc |
| | | | | ttatccaggctttttgacaacgctatgctcc |
| | | | | gcgccagaaaaaagagaCATGCTGAAGGGAC |
| | | | | CTTTACCAGTGATGTAAGTTCTTATTTGGAA |
| | | | | GGCCAAGCTGCCAAGGAATTCATTGCTTGGC |
| | | | | TGGTGAAAGGCCGAGGAagaaaaaagagaTA |
| | | | | CGCGGAAGGGACTTTCATCAGTGACTACAGT |
| | | | | ATTGCCATGGACAAGATTCACCAACAAGACT |
| | | | | TTGTGAACTGGCTGCTGGCCCAAAAGGGGAA |
| | | | | GAAGAATGACTGGAAACACAACATCACCCAG |
| | | | | TGA |
| 52 | GG_F | hInsul | hGLP- | ATGGCCCTGTGGATGCGCCTCCTGCCCCTGC |
| | | | 1, hGIP | TGGCGCTGCTGGCCCTCTGGGGACCTGACCC |
| | | | | AGCCGCAGCCtacccatacgatgttccagat |
| | | | | tacgctagaaaaaagagaCATGCTGAAGGGA |
| | | | | CCTTTACCAGTGATGTAAGTTCTTATTTGGA |
| | | | | AGGCCAAGCTGCCAAGGAATTCATTGCTTGG |
| | | | | CTGGTGAAAGGCCGAGGAagaaaaaagagaT |
| | | | | ACGCGGAAGGGACTTTCATCAGTGACTACAG |
| | | | | TATTGCCATGGACAAGATTCACCAACAAGAC |
| | | | | TTTGTGAACTGGCTGCTGGCCCAAAAGGGGA |
| | | | | AGAAGAATGACTGGAAACACAACATCACCCA |
| | | | | GTGA |
| 53 | GG_L | mGHRH | hGLP- | ATGCTGCTCTGGGTGCTCTTTGTGATCCTCA |
| | | | 1, hGIP | TCCTCACCAGTGGCTCCCACTGCTCActgcc |
| | | | | cccctcacctcccttcaggatgcagcgaCAT |
| | | | | GCTGAAGGGACCTTTACCAGTGATGTAAGTT |
| | | | | CTTATTTGGAAGGCCAAGCTGCCAAGGAATT |
| | | | | CATTGCTTGGCTGGTGAAAGGCCGAGGAaga |
| | | | | aaaaagagaTACGCGGAAGGGACTTTCATCA |
| | | | | GTGACTACAGTATTGCCATGGACAAGATTCA |
| | | | | CCAACAAGACTTTGTGAACTGGCTGCTGGCC |
| | | | | CAAAAGGGGAAGAAGAATGACTGGAAACACA |
| | | | | ACATCACCCAGTGA |
| 54 | GG_M | hAlbumin | hGLP- | ATGAAGTGGGTAACCTTTATTTCCCTTCTTT |
| | | | 1, hGIP | TTCTCTTTAGCTCGGCTTATTCCaggggtgt |
| | | | | gtttcgtcgaCATGCTGAAGGGACCTTTACC |
| | | | | AGTGATGTAAGTTCTTATTTGGAAGGCCAAG |
| | | | | CTGCCAAGGAATTCATTGCTTGGCTGGTGAA |
| | | | | AGGCCGAGGAagaaaaaagagaTACGCGGAA |
| | | | | GGGACTTTCATCAGTGACTACAGTATTGCCA |
| | | | | TGGACAAGATTCACCAACAAGACTTTGTGAA |
| | | | | CTGGCTGCTGGCCCAAAAGGGGAAGAAGAAT |
| | | | | GACTGGAAACACAACATCACCCAGTGA |
| 57 | 2xGLP-1 | n/a | hGLP- | CATGCTGAAGGGACCTTTACCAGTGATGTAA |
| | without | | 1, | GTTCTTATTTGGAAGGCCAAGCTGCCAAGGA |
| | signal | | hGLP-1 | ATTCATTGCTTGGCTGGTGAAAGGCCGAGGA |
| | peptide | | | agaaaaaagagaCATGCAGAGGGAACATTTA |
| | encoding | | | CTAGTGATGTCAGTTCATATCTTGAGGGACA |
| | sequence | | | AGCTGCTAAAGAATTTATTGCTTGGCTTGTG |
| | | | | AAGGGAAGAGGAtga |
| 58 | GG | n/a | hGLP- | CATGCTGAAGGGACCTTTACCAGTGATGTAA |
| | without | | 1, hGIP | GTTCTTATTTGGAAGGCCAAGCTGCCAAGGA |
| | signal | | | ATTCATTGCTTGGCTGGTGAAAGGCCGAGGA |
| | peptide | | | agaaaaaagagaTACGCGGAAGGGACTTTCA |
| | encoding | | | TCAGTGACTACAGTATTGCCATGGACAAGAT |
| | sequence | | | TCACCAACAAGACTTTGTGAACTGGCTGCTG |
| | | | | GCCCAAAAGGGGAAGAAGAATGACTGGAAAC |
| | | | | ACAACATCACCCAGTGA |

### Tricistronic Expression Constructs

In one aspect, provided is a tricistronic expression construct encoding a polyprotein, wherein:
a. the polyprotein comprises a signal peptide, a first gut peptide, a second gut peptide, and a third gut peptide; and
b. the polyprotein encoding sequence comprises:
   i. a sequence encoding the signal peptide
   ii. a sequence encoding the first gut peptide;
   iii. a sequence encoding the second gut peptide; and
   iv. a sequence encoding the third gut peptide.

In some embodiments, the first gut peptide, the second gut peptide, and/or the third gut peptide comprises a sequence selected from the group consisting of hGLP-1 peptide, hGIP peptide, hOXM peptide, PYY, hGlucagon peptide, and amylin peptide. In embodiments, the hGLP-1 peptide is the hGLP-1₇₋₃₇ peptide. In embodiments, the hGIP peptide is the hGIP₁₋₄₂ peptide. In some embodiments, the first gut peptide, the second gut peptide, and/or the third gut peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOS:1-5. In some embodiments, the first gut peptide, the second gut peptide, and/or the third gut peptide comprises a sequence that is at least 90% identical to any one of SEQ ID NOS:1-5. In some embodiments, the first gut peptide, the second gut peptide, and/or the third gut peptide comprises a sequence selected from SEQ ID NOS:1-5.

In some embodiments, the sequence encoding first gut peptide, the second gut peptide, and/or the third gut peptide comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOS:6-12. In some embodiments, the sequence encoding the first gut peptide, the second gut peptide, and/or the third gut peptide comprises a sequence that is at least 90% identical to any one of SEQ ID NOS:6-12. In some embodiments, the sequence encoding first gut peptide, the second gut peptide, and/or the third gut peptide comprises a sequence that is selected from SEQ ID NOS:6-12.

In some embodiments, the tricistronic expression construct encodes a sequence comprising a sequence disclosed in Table 9 or a portion of a sequence disclosed in Table 9. In some embodiments, the tricistronic expression construct comprises a sequence disclosed in Table 10 or a portion of a sequence disclosed in Table 10.

In some embodiments, the first gut peptide, the second gut peptide, and the third gut peptide are the same gut peptide. In some embodiments, the first gut peptide, the second gut peptide, and the third gut peptide are the same gut peptide, but the sequence encoding the first gut peptide, the sequence encoding the second gut peptide, and the sequence encoding the third gut peptide are different. In some embodiments, at least one of the sequence encoding the first gut peptide, the sequence encoding the second gut peptide, and the sequence encoding the third gut peptide is codon-optimized. In some embodiments, the sequence encoding the sequence encoding the first gut peptide, the sequence encoding the second gut peptide, and the sequence encoding the third gut peptide are codon-optimized.

In some embodiments, the first gut peptide, the second gut peptide and the third gut peptide are hGLP-1.

In some embodiments, the tricistronic expression construct encodes a sequence comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOS:59-61 or SEQ ID NO:75. In some embodiments, the tricistronic expression construct encodes a sequence comprising a sequence that is at least 90% identical to any one of SEQ ID NOS:59-61 or SEQ ID NO:75. In some embodiments, the tricistronic expression construct encodes a sequence comprising a sequence selected from SEQ ID NOS:59-61 or SEQ ID NO:75.

In some embodiments, the tricistronic expression construct comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOS:67-69 or SEQ ID NO:78. In some embodiments, the tricistronic expression construct comprises a sequence that is at least 90% identical to any one of SEQ ID NOS:67-69 or SEQ ID NO:78. In some embodiments, the tricistronic expression construct comprises a sequence selected from SEQ ID NOS:67-69 or SEQ ID NO:78.

In some embodiments, the first gut peptide, and the second gut peptide are different gut peptides. In some embodiments, the first gut peptide, the second gut peptide, and the third gut peptide are different gut peptides.

In some embodiments, the first gut peptide, the second gut peptide, and the third gut peptide are selected from the group consisting of (1) hGLP-1 peptide, hOXM peptide, and PYY or (2) hGLP-1 peptide, hGlucagon peptide, and hGIP peptide. In embodiments, the hGLP-1 peptide is the hGLP-1₇₋₃₇ peptide. In embodiments, the hGIP peptide is the hGIP₁₋₄₂ peptide.

In some embodiments, the tricistronic expression construct encodes a sequence comprising a sequence that is at least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOS:62-66 or SEQ ID NOS:76-77. In some embodiments, the tricistronic expression construct encodes a sequence comprising a sequence that is at least 90% identical to any one of SEQ ID NOS:62-66 or SEQ ID NOS:76-77. In some embodiments, the tricistronic expression construct encodes a sequence comprising any one of SEQ ID NOS:62-66 or SEQ ID NOS:76-77.

In some embodiments, the tricistronic expression construct comprises a sequence that is at least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOS:70-74 or SEQ ID NOS:79-80. In some embodiments, the tricistronic expression construct comprises a sequence that is at least 90% identical to any one of SEQ ID NOS:70-74 or SEQ ID NOS:79-80. In some embodiments, the tricistronic expression construct comprises any one of SEQ ID NOS:70-74 or SEQ ID NOS:79-80.

TABLE 9
Amino acid sequences of exemplary tricistronic expression constructs. hGLP-1 (SEQ ID NO: 1)
is shown in bold. hOXM (SEQ ID NO: 3) is underlined. PYY (SEQ ID NO: 4) is bold, underlined,
and in italics.

| SEQ | | | | |
|---|---|---|---|---|
| ID | Construct | Signal | Gut | |
| NO | name | peptide | peptides | Sequence |
| 59 | 3xGLP- | hAlbumin | hGLP-1, | MKWVTFISLLFLFSSAYSRGVFRRHAEGTFTSDVSSYLEGQAAKEFIAWLVKGR |
| | 1_3xB | | hGLP-1, | GRKKRHAEGTFTSDVSSYLEGQAAKEFIAWLVKGRGRKKRHAEGTFTSDVSSYL |
| | | | hGLP-1, | EGQAAKEFIAWLVKGRG |
| 60 | 3xGLP- | hInsul | hGLP-1, | MALWMRLLPLLALLALWGPDPAAAYPYDVPDYARKKRHAEGTFTSDVSSYLEGQ |
| | 1_3xC | | hGLP-1, | AAKEFIAWLVKGRGRKKRHAEGTFTSDVSSYLEGQAAKEFIAWLVKGRGRKKRH |
| | | | hGLP-1, | AEGTFTSDVSSYLEGQAAKEFIAWLVKGRG |
| 61 | 3xGLP- | mGHRH | hGLP-1, | MLLWVLFVILILTSGSHCSLPPSPPFRMQRHAEGTFTSDVSSYLEGQAAKEFIA |
| | 1_3xD | | hGLP-1, | WLVKGRGRKKRHAEGTFTSDVSSYLEGQAAKEFIAWLVKGRGRKKRHAEGTFTS |
| | | | hGLP-1, | DVSSYLEGQAAKEFIAWLVKGRG |
| 62 | GOP_J | hGH | hGLP-1, | MATGSRTSLLLAFGLLCLPWLQEGSAFPTIPLSRLEDNAMLRARKKRHAEGTFT |
| | | | hOXM, PYY | SDVSSYLEGQAAKEFIAWLVKGRGRRKRHSQGTFTSDYSKYLDSRRAQDEVqWL |
| | | | | MNTKRNRNNIARKKRIKPEAPREDASPEELNRYYASLRHYLNLVTRQRY |
| 63 | GOP_F | hInsul | hGLP-1, | MALWMRLLPLLALLALWGPDPAAAYPYDVPDYARKKRHAEGTFTSDVSSYLEGQ |
| | | | hOXM, PYY | AAKEFIAWLVKGRGRRKRHSQGTFTSDYSKYLDSRRAQDFVQWLMNTKRNRNNI |
| | | | | ARKKRIKPEAPREDASPEELNRYYASLRHYLNLVTRQRY |
| 64 | GOP_L | mGHRH | hGLP-1, | MLLWVLFVILILTSGSHCSLPPSPPFRMQRHAEGTFTSDVSSYLEGQAAKEFIA |
| | | | hOXM, PYY | WLVKGRGRRKRHSQGTFTSDYSKYLDSRRAQDFVQWLMNTKRNRNNIARKKRIK |
| | | | | PEAPREDASPEELNRYYASLRHYLNLVTRQRY |
| 65 | GOP_M | hAlbumin | hGLP-1, | MKWVTFISLLFLFSSAYSRGVFRRHAEGTFTSDVSSYLEGQAAKEFIAWLVKGR |
| | | | hOXM, PYY | GRRKRHSQGTFTSDYSKYLDSRRAQDFVQWLMNTKRNRNNIARKKRIKPEAPRE |
| | | | | DASPEELNRYYASLRHYLNLVTRQRY |
| 66 | GGG_A | hGH | hGLP-1, | MATGSRTSLLLAFGLLCLPWLQEGSAFPTIPLSRLEDNAMLRARKKRHAEGTFT |
| | | | hGlucagon, | SDVSSYLEGQAAKEFIAWLVKGRGRRKRHSQGTFTSDYSKYLDSRRAQDEVqWL |
| | | | hGIP | MNTRKKRYAEGTFISDYSIAMDKIHQQDFVNWLLAQKGKKNDWKHNITQ |
| 75 | 3xGLP-1 | n/a | hGLP-1, | HAEGTFTSDVSSYLEGQAAKEFIAWLVKGRGRKKRHAEGTFTSDVSSYLEGQAA |
| | w/o signal | | hGLP-1, | KEFIAWLVKGRGRKKRHAEGTFTSDVSSYLEGQAAKEFIAWLVKGRG |
| | peptide | | hGLP-1, | |
| 76 | hGLP-1, | n/a | hGLP-1, | HAEGTFTSDVSSYLEGQAAKEFIAWLVKGRGRRKRHSQGTFTSDYSKYLDSRRA |
| | hOXM, | | hOXM, PYY | QDFVQWLMNTKRNRNNIARKKRIKPEAPREDASPEELNRYYASLRHYLNLVTRQ |
| | PYY w/o | | | RY |
| | signal | | | |
| | peptide | | | |
| 77 | hGLP-1, | n/a | hGLP-1, | HAEGTFTSDVSSYLEGQAAKEFIAWLVKGRGRRKRHSQGTFTSDYSKYLDSRRA |
| | hGlucagon, | | hGlucagon, | QDFVQWLMNTRKKRYAEGTFISDYSIAMDKIHQQDFVNWLLAQKGKKNDWKHNI |
| | hGIP w/o | | hGIP | TQ |
| | signal | | | |
| | peptide | | | |

TABLE 10
Nucleic acid sequences of exemplary tricistronic expression constructs. Signal peptide encoding
sequence is shown in italic upper-case letters. Furin cleavage site encoding sequence is shown
in lower case letters. Gut peptide encoding sequences are shown in upper case letters
(not italic).

| SEQ | | | | |
|---|---|---|---|---|
| ID | Construct | Signal | Gut | |
| NO | name | peptide | peptides | Sequence |
| 67 | 3xGLP- | hAlbumin | hGLP-1, | ATGAAGTGGGTAACCTTTATTTCCCTTCTTTTTCTCTTTAGCTCGGCTTATTCC |
| | 1_3xB | | hGLP-1, | aggggtgtgtttcgtcgaCATGCTGAAGGGACCTTTACCAGTGATGTAAGTTCT |
| | | | hGLP-1, | TATTTGGAAGGCCAAGCTGCCAAGGAATTCATTGCTTGGCTGGTGAAAGGCCGA |
| | | | | GGAagaaaaaagagaCATGCAGAGGGAACATTTACTAGTGATGTCAGTTCATAT |
| | | | | CTTGAGGGACAAGCTGCTAAAGAATTTATTGCTTGGCTTGTGAAGGGAAGAGGA |
| | | | | agaaagaagagaCATGCTGAAGGGACATTTACCTCAGATGTTTCTTCATACCTG |
| | | | | GAAGGACAGGCTGCCAAGGAATTTATTGCATGGCTTGTGAAAGGCAGGGGCtga |
| 68 | 3xGLP- | hInsul | hGLP-1, | ATGGCCCTGTGGATGCGCCTCCTGCCCCTGCTGGCGCTGCTGGCCCTCTGGGGA |
| | 1_3xC | | hGLP-1, | CCTGACCCAGCCGCAGCCtacccatacgatgttccagattacgctagaaaaaag |
| | | | hGLP-1, | agaCATGCTGAAGGGACCTTTACCAGTGATGTAAGTTCTTATTTGGAAGGCCAA |
| | | | | GCTGCCAAGGAATTCATTGCTTGGCTGGTGAAAGGCCGAGGAagaaaaaagaga |
| | | | | CATGCAGAGGGAACATTTACTAGTGATGTCAGTTCATATCTTGAGGGACAAGCT |
| | | | | GCTAAAGAATTTATTGCTTGGCTTGTGAAGGGAAGAGGAagaaagaagagaCAT |
| | | | | GCTGAAGGGACATTTACCTCAGATGTTTCTTCATACCTGGAAGGACAGGCTGCC |
| | | | | AAGGAATTTATTGCATGGCTTGTGAAAGGCAGGGGCtga |
| 69 | 3xGLP- | mGHRH | hGLP-1, | ATGCTGCTCTGGGTGCTCTTTGTGATCCTCATCCTCACCAGTGGCTCCCACTGC |
| | 1_3xD | | hGLP-1, | TCActgcccccctcacctcccttcaggatgcagcgaCATGCTGAAGGGACCTTT |
| | | | hGLP-1, | ACCAGTGATGTAAGTTCTTATTTGGAAGGCCAAGCTGCCAAGGAATTCATTGCT |
| | | | | TGGCTGGTGAAAGGCCGAGGAagaaaaaagagaCATGCAGAGGGAACATTTACT |
| | | | | AGTGATGTCAGTTCATATCTTGAGGGACAAGCTGCTAAAGAATTTATTGCTTGG |
| | | | | CTTGTGAAGGGAAGAGGAagaaagaagagaCATGCTGAAGGGACATTTACCTCA |
| | | | | GATGTTTCTTCATACCTGGAAGGACAGGCTGCCAAGGAATTTATTGCATGGCTT |
| | | | | GTGAAAGGCAGGGGCtga |
| 70 | GOP_J | hGH | hGLP-1, | ATGGCTACAGGCTCCCGGACGTCCCTGCTCCTGGCTTTTGGCCTGCTCTGCCTG |
| | | | hOXM, PYY | CCCTGGCTTCAAGAGGGCAGTGCCttcccaaccattcccttatccaggcttttt |
| | | | | gacaacgctatgctccgcgccagaaaaaagagaCATGCTGAAGGGACCTTTACC |
| | | | | AGTGATGTAAGTTCTTATTTGGAAGGCCAAGCTGCCAAGGAATTCATTGCTTGG |
| | | | | CTGGTGAAAGGCCGAGGAagacgtaagaggCATTCACAGGGCACATTCACCAGT |
| | | | | GACTACAGCAAGTATCTGGACTCCAGGCGTGCCCAAGATTTTGTGCAGTGGTTG |
| | | | | ATGAATACCAAGAGGAACAGGAATAACATTGCCagaaaaaagagaATCAAACCC |
| | | | | GAGGCTCCCCGCGAAGACGCCTCGCCGGAGGAGCTGAACCGCTACTACGCCTCC |
| | | | | CTGCGCCACTACCTCAACCTGGTCACCCGGCAGCGGTATTGA |
| 71 | GOP_F | hInsul | hGLP-1, | ATGGCCCTGTGGATGCGCCTCCTGCCCCTGCTGGCGCTGCTGGCCCTCTGGGGA |
| | | | hOXM, PYY | CCTGACCCAGCCGCAGCCtacccatacgatgttccagattacgctagaaaaaag |
| | | | | agaCATGCTGAAGGGACCTTTACCAGTGATGTAAGTTCTTATTTGGAAGGCCAA |
| | | | | GCTGCCAAGGAATTCATTGCTTGGCTGGTGAAAGGCCGAGGAagacgtaagagg |
| | | | | CATTCACAGGGCACATTCACCAGTGACTACAGCAAGTATCTGGACTCCAGGCGT |
| | | | | GCCCAAGATTTTGTGCAGTGGTTGATGAATACCAAGAGGAACAGGAATAACATT |
| | | | | GCCagaaaaaagagaATCAAACCCGAGGCTCCCCGCGAAGACGCCTCGCCGGAG |
| | | | | GAGCTGAACCGCTACTACGCCTCCCTGCGCCACTACCTCAACCTGGTCACCCGG |
| | | | | CAGCGGTATTGA |
| 72 | GOP_L | mGHRH | hGLP-1, | ATGCTGCTCTGGGTGCTCTTTGTGATCCTCATCCTCACCAGTGGCTCCCACTGC |
| | | | hOXM, PYY | TCActgcccccctcacctcccttcaggatgcagcgaCATGCTGAAGGGACCTTT |
| | | | | ACCAGTGATGTAAGTTCTTATTTGGAAGGCCAAGCTGCCAAGGAATTCATTGCT |
| | | | | TGGCTGGTGAAAGGCCGAGGAagacgtaagaggCATTCACAGGGCACATTCACC |
| | | | | AGTGACTACAGCAAGTATCTGGACTCCAGGCGTGCCCAAGATTTTGTGCAGTGG |
| | | | | TTGATGAATACCAAGAGGAACAGGAATAACATTGCCagaaaaaagagaATCAAA |
| | | | | CCCGAGGCTCCCCGCGAAGACGCCTCGCCGGAGGAGCTGAACCGCTACTACGCC |
| | | | | TCCCTGCGCCACTACCTCAACCTGGTCACCCGGCAGCGGTATTGA |
| 73 | GOP_M | hAlbumin | hGLP-1, | ATGAAGTGGGTAACCTTTATTTCCCTTCTTTTTCTCTTTAGCTCGGCTTATTCC |
| | | | hOXM, PYY | aggggtgtgtttcgtcgaCATGCTGAAGGGACCTTTACCAGTGATGTAAGTTCT |
| | | | | TATTTGGAAGGCCAAGCTGCCAAGGAATTCATTGCTTGGCTGGTGAAAGGCCGA |
| | | | | GGAagacgtaagaggCATTCACAGGGCACATTCACCAGTGACTACAGCAAGTAT |
| | | | | CTGGACTCCAGGCGTGCCCAAGATTTTGTGCAGTGGTTGATGAATACCAAGAGG |
| | | | | AACAGGAATAACATTGCCagaaaaaagagaATCAAACCCGAGGCTCCCCGCGAA |
| | | | | GACGCCTCGCCGGAGGAGCTGAACCGCTACTACGCCTCCCTGCGCCACTACCTC |
| | | | | AACCTGGTCACCCGGCAGCGGTATTGA |
| 74 | GGG_A | hGH | hGLP-1, | ATGGCTACAGGCTCCCGGACGTCCCTGCTCCTGGCTTTTGGCCTGCTCTGCCTG |
| | | | hGlucagon, | CCCTGGCTTCAAGAGGGCAGTGCCttcccaaccattcccttatccaggcttttt |
| | | | hGIP | gacaacgctatgctccgcgccagaaaaaagagaCATGCTGAAGGGACCTTTACC |
| | | | | AGTGATGTAAGTTCTTATTTGGAAGGCCAAGCTGCCAAGGAATTCATTGCTTGG |
| | | | | CTGGTGAAAGGCCGAGGAagacgtaagaggCATTCACAGGGCACATTCACCAGT |
| | | | | GACTACAGCAAGTATCTGGACTCCAGGCGTGCCCAAGATTTTGTGCAGTGGTTG |
| | | | | ATGAATACCagaaaaaagagaTACGCGGAAGGGACTTTCATCAGTGACTACAGT |
| | | | | ATTGCCATGGACAAGATTCACCAACAAGACTTTGTGAACTGGCTGCTGGCCCAA |
| | | | | AAGGGGAAGAAGAATGACTGGAAACACAACATCACCCAGTGA |
| 78 | 3xGLP-1 | n/a | hGLP-1, | CATGCTGAAGGGACCTTTACCAGTGATGTAAGTTCTTATTTGGAAGGCCAAGCT |
| | w/o signal | | hGLP-1, | GCCAAGGAATTCATTGCTTGGCTGGTGAAAGGCCGAGGAagaaaaaagagaCAT |
| | peptide | | hGLP-1, | GCAGAGGGAACATTTACTAGTGATGTCAGTTCATATCTTGAGGGACAAGCTGCT |
| | | | | AAAGAATTTATTGCTTGGCTTGTGAAGGGAAGAGGA |
| | | | | agaaagaagagaCATGCTGAAGGGACATTTACCTCAGATGTTTCTTCATACCTG |
| | | | | GAAGGACAGGCTGCCAAGGAATTTATTGCATGGCTTGTGAAAGGCAGGGGCtga |
| 79 | hGLP-1, | n/a | hGLP-1, | CATGCTGAAGGGACCTTTACCAGTGATGTAAGTTCTTATTTGGAAGGCCAAGCT |
| | hOXM, | | hOXM, PYY | GCCAAGGAATTCATTGCTTGGCTGGTGAAAGGCCGAGGAagacgtaagaggCAT |
| | PYY w/o | | | TCACAGGGCACATTCACCAGTGACTACAGCAAGTATCTGGACTCCAGGCGTGCC |
| | signal | | | CAAGATTTTGTGCAGTGGTTGATGAATACCAAGAGGAACAGGAATAACATTGCC |
| | peptide | | | agaaaaaagagaATCAAACCCGAGGCTCCCCGCGAAGACGCCTCGCCGGAGGAG |
| | | | | CTGAACCGCTACTACGCCTCCCTGCGCCACTACCTCAACCTGGTCACCCGGCAG |
| | | | | CGGTATTGA |
| 80 | hGLP-1, | n/a | hGLP-1, | CATGCTGAAGGGACCTTTACCAGTGATGTAAGTTCTTATTTGGAAGGCCAAGCT |
| | hGlucagon, | | hGlucagon, | GCCAAGGAATTCATTGCTTGGCTGGTGAAAGGCCGAGGAagacgtaagaggCAT |
| | hGIP w/o | | hGIP | TCACAGGGCACATTCACCAGTGACTACAGCAAGTATCTGGACTCCAGGCGTGCC |
| | signal | | | CAAGATTTTGTGCAGTGGTTGATGAATACCagaaaaaagagaTACGCGGAAGGG |
| | peptide | | | ACTTTCATCAGTGACTACAGTATTGCCATGGACAAGATTCACCAACAAGACTTT |
| | | | | GTGAACTGGCTGCTGGCCCAAAAGGGGAAGAAGAATGACTGGAAACACAACATC |
| | | | | ACCCAGTGA |

### Furin Recognition and Cleavage Sequences

Provided herein are expression constructs that encode one or more sequences that are recognized by a protease. In embodiments, the protease is furin. Furin cleaves proteins just downstream of a basic amino acid minimal furin cleavage site. In embodiments, this minimal furin cleavage site is Arg-X-X-Arg (preferably, Arg-X-(Arg/Lys)-Arg). However, furin may recognize a longer sequence within the target polypeptide in addition to the minimal furin cleavage site. This longer sequence (comprising the minimal furin cleavage site) is referred to herein as a “furin recognition and cleavage sequence.” The inclusion of a furin recognition and cleavage sequence can promote the functional N-terminus of expressed polypeptide (such as a gut peptide or a polyprotein comprising one or more gut peptides) to be fully processed and generated in non-endocrine cells.

In some embodiments, the furin recognition and cleavage sequence comprises (1) any one of SEQ ID NOs:89, 92-96 or (2) a sequence that is at least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOs:89, 92-96. In some embodiments, the furin recognition and cleavage sequence comprises a portion of (1) any one of SEQ ID NOs:89, 92-96 or (2) a sequence that is at least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOs:89, 92-96.

In embodiments, provided is an expression construct comprising a sequence encoding any one of SEQ ID NOs:89, 92-96 or a sequence that is least 80%, at least 85%, at least 90%, at least 91%, at least 92%, at least 93%, at least 94%, at least 95%, at least 96%, at least 91%, at least 97%, at least 98%, or at least 99% identical to any one of SEQ ID NOs:89, 92-96.

In embodiments, provided is an expression construct comprising a sequence encoding any one of sequences RKKR (SEQ ID NO:97), RMQR (SEQ ID NO:98), VFRR (SEQ ID NO:99), or RKKR (SEQ ID NO:100).

In embodiments, the monocistronic, bicistronic, or tricistronic expression construct comprises a sequence disclosed in Table 11 or a portion of a sequence disclosed in Table 11. In embodiments, the monocistronic, bicistronic, or tricistronic expression construct comprises a sequence encoding a sequence disclosed in Table 11 or a portion of a sequence disclosed in Table 11.

Provided herein are expression constructs comprising one or more sequences that are least 80%, at least 85%, at least 90%, at least 91%, at least 92%, at least 93%, at least 94%, at least 95%, at least 96%, at least 91%, at least 97%, at least 98%, or at least 99% identical to of any of the sequences disclosed herein. Provided herein are expression constructs comprising one or more sequences that comprise a portion of any of the sequences disclosed herein.

Provided herein are expression constructs encoding for one or more sequences that are least 80%, at least 85%, at least 90%, at least 91%, at least 92%, at least 93%, at least 94%, at least 95%, at least 96%, at least 91%, at least 97%, at least 98%, or at least 99% identical to of any of the sequences disclosed herein. Provided herein are expression constructs encoding for one or more sequences that comprise a portion of any of the sequences disclosed herein.

TABLE 11
Selected sequences that facilitate furin
cleavage.

| SEQ | |
|---|---|
| ID NO | Nucleic Acid Sequence |
| 81 | TACCCATACGATGTTCCAGATTACGCTAG |
| | AAAAAAGAGA |
| 84 | TTCCCAACCATTCCCTTATCCAGGCTTTT |
| | TGACAACGCTATGCTCCGCGCCAGAAAAA |
| | AGAGA |
| 85 | GCTCCCCCACGCCTCATCTGCGACAGTCG |
| | AGTTCTGGAGAGGTACAGAAAAAAGAGA |
| 86 | CTGCCCCCCTCACCTCCCTTCAGGATGCA |
| | GCGA |
| 87 | AGGGGTGTGTTTCGTCGA |
| 88 | ACAGTTTTTCTTGATCATGAAAACGCCAA |
| | CAAAATTCTGAATCGGCCAAAGAGG |
| 89 | YPYDVPDYARKKR |
| 92 | FPTIPLSRLEDNAMLRARKK |
| | R |
| 93 | APPRLICDSRVLERYRKKR |
| 94 | LPPSPPFRMQR |
| 95 | RGVFRR |
| 96 | TVELDHENANKILNRPKR |
| 97 | RKKR |
| 98 | RMQR |
| 99 | VERR |
| 100 | RPKR |

### Leader Sequences

In embodiments, the expression constructs disclosed herein comprise a leader sequence. As used herein, a “leader sequence” is a sequence that comprises (1) a signal peptide and a protease recognition and cleavage sequence and/or (2) a signal peptide and a minimal protease cleavage site. In embodiments, a leader sequence comprises (1) a signal peptide and a furin recognition and cleavage sequence and/or (2) a signal peptide and a minimal furin cleavage site. The inclusion of a furin recognition and cleavage sequence in the leader sequence can promote the functional N-terminus of expressed polypeptide (such as a gut peptide or a polyprotein comprising one or more gut peptides) to be fully processed and generated in non-endocrine cells.

The leader sequence may be derived from a naturally occurring, secreted polypeptide or from a variant of a naturally occurring, secreted polypeptide. In embodiments, the leader sequence, or a portion thereof, is derived from influenza virus hemagglutinin, human growth hormone, murine growth hormone-releasing hormone, or human albumin.

### Promoters

Any suitable promoter may be used in the expression constructs disclosed herein. In some embodiments, the promoter is a CMV or a CASI promoter.

### Regulation of Gene Expression

In some embodiments, the expression constructs disclosed herein provide for constitutive expression of the polypeptides disclosed herein.

In some embodiments, the expression constructs disclosed herein provide for regulatable expression of the polypeptides disclosed herein.

In embodiments, the sequence encoding a polypeptide disclosed herein comprises a riboswitch comprising an aptamer, wherein the riboswitch is operable linked to the sequence encoding the polypeptide. In embodiments the sequence encoding a polypeptide disclosed herein comprises a gene regulation cassette, wherein the gene regulation cassette comprises an aptamer. In embodiments the polypeptide is a polyprotein disclosed herein.

Aptamers are single-stranded nucleic acid molecules that non-covalently bind to specific ligands with high affinity and specificity by folding into three-dimensional structures. Aptamer ligands include ions, small molecules, proteins, viruses, and cells. Aptamer ligands can be, for example, an organic compound, amino acid, steroid, carbohydrate, or nucleotide. Non-limiting examples of small molecule aptamer ligands include antibiotics, therapeutics, dyes, cofactors, metabolites, molecular markers, neurotransmitters, pollutants, toxins, food adulterants, carcinogens, drugs of abuse. As such, aptamers are useful for the detection of small molecules. Application of small-molecule detection by aptamers include environmental monitoring, food safety, medicine (including diagnostics), microbiology, analytical chemistry, forensic science, agriculture, and basic biology research. The term “aptamer” as used herein refers to an RNA polynucleotide (or DNA sequence encoding the RNA polynucleotide) that specifically binds to a class of ligands. The term “ligand” refers to a molecule that is specifically bound by an aptamer. Aptamers have binding regions that are capable of forming complexes with an intended target molecule (i.e., the ligand). An aptamer will typically be between about 15 and about 200 nucleotides in length. More commonly, an aptamer will be between about 30 and about 100 nucleotides in length, for example, 70 to 90 nucleotides in length. Aptamers typically comprise multiple paired (P) regions in which the aptamer forms a stem and unpaired regions where the aptamer forms a joining (J) region or a loop (L) region. The paired regions can be numbered sequentially starting at the 5′ end (P1) and numbering each stem sequentially (P2, P3, etc.). The loops (L1, L2, etc.) are numbered based on the adjacent paired region and the joining regions are numbered according to the paired regions that they link. Aptamers are oligonucleotides that bind to a target ligand with high affinity and specificity.

In embodiments, the presence of a small molecule that binds to an aptamer leads to an increase in expression of a sequence encoding a polypeptide disclosed herein as compared to the expression of the sequence encoding a polypeptide disclosed herein in absence of the small molecule. In such an embodiment, the aptamer constitutes an “on” switch. In embodiments, the expression of a sequence encoding a polypeptide disclosed herein is increased by at least 3-fold, by at least 5-fold, by at least 10-fold, by at least 15-fold, by at least 20-fold, by at least 25-fold, by at least 30-fold, by at least 40-fold, by at least 50-fold, by at least 100-fold, by at least 1000-fold, or by at least 10,000-fold in presence of the small molecule that binds to an aptamer as compared to in absence of the small molecule. In embodiments, the expression of a sequence encoding a polypeptide disclosed herein is increased by between 2-fold and 10-fold, between 5-fold and 10-fold, between 5-fold and 15-fold, between 5-fold and 20-fold, between 5-fold and 25-fold, between 5-fold and 30-fold, between 10-fold and 20-fold, between 10-fold and 30-fold, between 10-fold and 40-fold, between 10-fold and 50-fold, between 10-fold and 100-fold, between 10-fold and 500-fold, between 10-fold and 1,000-fold, between 50-fold and 100-fold, between 50-fold and 500-fold, between 50-fold and 100-fold, between 50-fold and 1,000-fold, between 100-fold and 1,000-fold, or between 100-fold and 10,000-fold in presence of the small molecule that binds to an aptamer as compared to in absence of the small molecule.

In embodiments, the presence of a small molecule that binds to an aptamer leads to a decrease in expression of a sequence encoding a polypeptide disclosed herein as compared to the expression of the sequence encoding a polypeptide disclosed herein in the absence of the small molecule. In such embodiments, the aptamer constitutes an “off” switch. In embodiments, the expression of the sequence encoding a polypeptide disclosed herein is decreased by at least 3-fold, by at least 5-fold, by at least 10-fold, by at least 15-fold, by at least 20-fold, by at least 25-fold, by at least 30-fold, by at least 40-fold, by at least 50-fold, by at least 100-fold, by at least 1000-fold, or by at least 10,000-fold in presence of the small molecule that binds to an aptamer as compared to in absence of the small molecule. In one embodiment, the expression of the sequence encoding a polypeptide disclosed herein is decreased by between 2-fold and 10-fold, between 5-fold and 10-fold, between 5-fold and 15-fold, between 5-fold and 20-fold, between 5-fold and 25-fold, between 5-fold and 30-fold, between 10-fold and 20-fold, between 10-fold and 30-fold, between 10-fold and 40-fold, between 10-fold and 50-fold, between 10-fold and 100-fold, between 10-fold and 500-fold, between 10-fold and 1,000-fold, between 50-fold and 100-fold, between 50-fold and 500-fold, between 50-fold and 100-fold, between 50-fold and 1,000-fold, between 100-fold and 1,000-fold, or between 100-fold and 10,000-fold in presence of the small molecule that binds to an aptamer as compared to in absence of the small molecule.

In embodiments, the aptamer is part of a riboswitch. Riboswitches are regulatory segments of an RNA polynucleotide that regulate the stability of the RNA polynucleotide and/or regulate the production of a protein from the RNA polynucleotide in response to the presence or absence of aptamer-specific ligand molecules. In embodiments, the riboswitch comprises a sensor region (e.g., the aptamer region) and an effector region that together are responsible for sensing the presence of a ligand (e.g., a small molecule) and causing an effect that leads to increased or decreased expression of the sequence encoding a polypeptide disclosed herein. The riboswitches described herein are recombinant, utilizing polynucleotides from two or more sources. In embodiments, the sensor and effector regions are joined by a polynucleotide linker. In embodiments, the polynucleotide linker forms an RNA stem or paired region (i.e., a region of the RNA polynucleotide that is double-stranded). In embodiments, the paired region linking the aptamer to the effector region comprises all, or some of an aptamer stem (e.g., for example all, or some of the aptamer P1 stem).

Riboswitches comprising aptamer sequences may be used, for example, to control the formation of rho-independent transcription termination hairpins leading to premature transcription termination. Riboswitches comprising aptamer sequences may also induce structural changes in the RNA, leading to sequestration for the ribosome binding site and inhibition of translation. Alternative riboswitch structures comprising the aptamer sequences disclosed herein can further affect the splicing of mRNA in response to the presence of the small molecule ligand.

### Alternative Splicing Riboswitch

In one embodiment, the riboswitches described herein are encoded as part of a gene regulation cassette for the regulation of a sequence encoding a polypeptide disclosed herein by aptamer/ligand mediated alternative splicing of the resulting RNA (e.g., pre-mRNA). In this context, the gene regulation cassette comprises a riboswitch comprising a sensor region (e.g., the aptamers described herein) and an effector region that together are responsible for sensing the presence of a small molecule ligand and altering splicing to an alternative exon. Splicing refers to the process by which an intronic sequence is removed from the nascent pre-messenger RNA (pre-mRNA) and the exons are joined together to form the mRNA. Splice sites are junctions between exons and introns and are defined by different splice site consensus sequences at the 5′ and 3′ ends of the intron (i.e., the splice donor and splice acceptor sites, respectively). Splicing is carried out by a large multi-component structure called the spliceosome, which is a collection of small nuclear ribonucleoproteins (snRNPs) and a diverse array of auxiliary proteins. By recognizing various cis regulatory sequences, the spliceosome defines exon/intron boundaries, removes intronic sequences, and splices together the exons into a final message (e.g., the mRNA). In the case of alternative splicing, certain exons can be included or excluded to vary the final coding message thereby changing the resulting expressed protein.

In one embodiment, the regulation of a sequence encoding a polypeptide disclosed herein expression is achieved by using any of the DNA constructs disclosed in PCT Patent Publication WO2016/126747, which is hereby incorporated by reference in its entirety. In embodiments of the present disclosure, the riboswitches and polynucleotide cassettes disclosed in PCT Patent Publication WO2016/126747 comprise an aptamer sequence described herein in place of the aptamer sequence disclosed in PCT Patent Publication WO2016/126747.

In one embodiment, the polynucleotide cassette comprises (a) a riboswitch and (b) an alternatively-spliced exon, flanked by a 5′ intron and a 3′ intron, wherein the riboswitch comprises (i) an effector region comprising a stem forming sequence that includes the 5′ splice site sequence of the 3′ intron and sequence complementary to the 5′ splice site sequence of the 3′ intron, and (ii) an aptamer. In embodiments, the effector region comprises the intronic 5′ splice site (“5′ ss”) sequence of the intron that is immediately 3′ of the alternative exon, as well as the sequence complimentary to the 5′ ss sequence of the 3′ intron. When the aptamer binds its ligand, the effector region forms a stem and thus prevents splicing to the splice donor site at the 3′ end of the alternative exon. Under certain conditions (for example, when the aptamer is not bound to its ligand), the effector region is in a context that provides access to the splice donor site at the 3′ end of the alternative exon, leading to inclusion of the alternative exon in the mRNA of the sequence encoding a polypeptide disclosed herein. In some embodiments, the polynucleotide cassette is placed in the sequence encoding a polypeptide disclosed herein gene to regulate expression of the sequence encoding a polypeptide disclosed herein in response to a ligand. In one embodiment, the alternatively-spliced exon comprises a stop codon that is in-frame with the sequence encoding a polypeptide disclosed herein when the alternatively-spliced exon is spliced into the mRNA of the sequence encoding a polypeptide disclosed herein.

In one embodiment, the gene regulation cassette comprises the sequence of “SEQ ID NO:101-X-SEQ ID NO:106,” wherein —X— represents an aptamer sequence. Lower case letters indicate paired stem sequence linking the aptamer to the remainder of the riboswitch. In one embodiment, the alternative exon (underlined in SEQ ID NO:101, below) is replaced with another alternative exon sequence.

(SEQ ID NO: 101)
GTGAGTCTATGGGACCCTTGATGTTTTCTTTCCCCTTCTTTTCTATGGTTAA
GTTCATGTCATAGGAAGGGGAGAAGTAACAGGGTACACATATTGACCAAATCAGGG
TAATTTTGCATTTGTAATTTTAAAAAATGCTTTCTTCTTTTAATATACTTTTTTGTTTA
TCTTATTTCTAATACTTTCCCTAATCTCTTTCTTTCAGGGCAATAATGATACAATGTA
TCATGCCGAGTAACGCTGTTTCTCTAACTTGTAGGAATGAATTCAGATATTTCCAGA
GAATGAAAAAAAAATCTTCAGTAGAAGgtaatgt-X-
(SEQ ID NO: 116)
acattacGCACCATTCTAAAGAATAACAGTGATAATTTCTGGGTTAAGGCAATAGCAAT
ATTTCTGCATATAAATATTTCTGCATATAAATTGTAACTGATGTAAGAGGTTTCATAT
TGCTAATAGCAGCTACAATCCAGCTACCATTCTGCTTTTATTTTATGGTTGGGATAAG
GCTGGATTATTCTGAGTCCAAGCTAGGCCCTTTTGCTAATCATGTTCATACCTCTTAT
CTTCCTCCCACAG

The alternative exon is flanked by 5′ and 3′ intronic sequences. The 5′ and 3′ intronic sequences that can be used in the gene regulation cassettes disclosed herein can be any sequence that can be spliced out of the sequence encoding a polypeptide disclosed herein creating either the mRNA of the sequence encoding a polypeptide disclosed herein or the sequence encoding a polypeptide disclosed herein comprising the alternative exon in the mRNA, depending upon the presence or absence of a ligand that binds the aptamer. The 5′ and 3′ intronic sequences each have the sequences necessary for splicing to occur, i.e., splice donor, splice acceptor and branch point sequences. In one embodiment, the 5′ and 3′ intronic sequences of the gene regulation cassette are derived from one or more naturally occurring introns or portions thereof. In one embodiment, the 5′ and 3′ intronic sequences are derived from a truncated human beta-globin intron 2 (IVS2Δ), from intron 2 of the human beta-globin gene, from the SV40 mRNA intron (used in pCMV-LacZ vector from Clontech Laboratories, Inc.), from intron 6 of human triose phosphate isomerase (TPI) gene (Nott Ajit, et al. RNA. 2003, 9:6070617), from an intron from human factor IX (Sumiko Kurachi, et al. J. Bio. Chem. 1995, 270(10), 5276), or from any genomic fragment or synthetic introns (Yi Lai, et al. Hum Gene Ther. 2006:17(10): 1036) that contain elements that are sufficient for regulated splicing (Thomas A. Cooper, Methods 2005 (37):331).

The splice donor and splice acceptor sites in the alternative splicing gene regulation cassette can be modified to be strengthened or weakened. That is, the splice site sequences can be modified to be closer to the consensus for a splice donor or acceptor by standard cloning methods, site directed mutagenesis, and the like. Splice site sequences that are more similar to the splice consensus sequence tend to promote splicing and are thus strengthened. Splice site sequences that are less similar to the splice consensus sequence tend to hinder splicing and are thus weakened. The consensus for the splice donor of the most common class of introns (U2) is A/C A G∥G T A/G A G T (SEQ ID NO:102, where II denotes the exon/intron boundary). The consensus for the splice acceptor is C A G∥G (where II denotes the exon/intron boundary). The frequency of particular nucleotides at the splice donor and acceptor sites are described in the art (see, e.g., Zhang, M. Q., Hum Mol Genet. 1988. 7(5):919-932). The strength of 5′ and 3′ splice sites can be adjusted to modulate splicing of the alternative exon.

Additional modifications to 5′ and 3′ introns present in the alternative splicing gene regulation cassette that can be made to modulate splicing include modifying, deleting, and/or adding intronic splicing enhancer elements, intronic splicing suppressor elements and or splice sites, and/or modifying the branch site sequence.

In one embodiment, the 5′ intron has been modified to contain a stop codon that will be in frame with the sequence encoding a polypeptide disclosed herein. The 5′ and 3′ intronic sequences can also be modified to remove cryptic slice sites, which can be identified with publicly available software (see, e.g., Kapustin, Y. et al. Nucl. Acids Res. 2011. 1-8).

The lengths of the 5′ and 3′ intronic sequences can be adjusted in order to, for example, meet the size requirements for viral expression constructs. In one embodiment, the 5′ and/or 3′ intronic sequences are about 50 to about 300 nucleotides in length. In one embodiment, the 5′ and/or 3′ intronic sequences are about 125 to about 240 nucleotides in length.

The stem portion of the effector region should be of a sufficient length (and GC content) to substantially prevent alternative splicing of the alternative exon upon ligand binding the aptamer, while also allowing access to the splice site when the ligand is not present in sufficient quantities. In embodiments, the stem portion of the effector region comprises a stem sequence in addition to the 5′ splice site sequence of the 3′ intron and its complementary sequence of the 5′ splice site sequence. In embodiments, this additional stem sequence comprises a sequence from the aptamer stem. The length and sequence of the stem portion can be modified using known techniques in order to identify stems that allow acceptable background expression of the sequence encoding a polypeptide disclosed herein when no ligand is present and acceptable expression levels of the sequence encoding a polypeptide disclosed herein when the ligand is present. In one embodiment, the effector region stem of the riboswitch is about 7 to about 20 base pairs in length. In one embodiment, the effector region stem is 8 to 11 base pairs in length. In addition to the length of the stem, the GC base pair content of the stem can be altered to modify the stability of the stem.

In one embodiment, the alternative exon that is part of the alternative splicing gene regulation cassettes disclosed herein is a polynucleotide sequence capable of being transcribed to a pre-mRNA and alternatively spliced into the mRNA of the sequence encoding a polypeptide disclosed herein. In one embodiment, the alternative exon contains at least one sequence that inhibits translation such that when the alternative exon is included in the mRNA of the sequence encoding a polypeptide disclosed herein, expression of the sequence encoding a polypeptide disclosed herein from that mRNA is prevented or reduced. In a preferred embodiment, the alternative exon contains a stop codon (TGA, TAA, TAG) that is in frame with the sequence encoding a polypeptide disclosed herein when the alternative exon is included in the mRNA of the sequence encoding a polypeptide disclosed herein by splicing. In embodiments, the alternative exon comprises, in addition to a stop codon, or as an alternative to a stop codon, another sequence that reduces or substantially prevents translation when the alternative exon is incorporated by splicing into the mRNA of the sequence encoding a polypeptide disclosed herein including, e.g., a microRNA binding site, which leads to degradation of the mRNA. In one embodiment, the alternative exon comprises a miRNA binding sequence that results in degradation of the mRNA. In one embodiment, the alternative exon encodes a polypeptide sequence which reduces the stability of the protein containing this polypeptide sequence. In one embodiment, the alternative exon encodes a polypeptide sequence which directs the protein containing this polypeptide sequence for degradation.

The basal or background level of splicing of the alternative exon can be optimized by altering exon splice enhancer (ESE) sequences and exon splice suppressor (ESS) sequences and/or by introducing ESE or ESS sequences into the alternative exon. Such changes to the sequence of the alternative exon can be accomplished using methods known in the art, including, but not limited to site directed mutagenesis. Alternatively, oligonucleotides of a desired sequence (e.g., comprising all or part of the alternative exon) can be obtained from commercial sources and cloned into the gene regulation cassette. Identification of ESS and ESE sequences can be accomplished by methods known in the art, including, for example using ESEfinder 3.0 (Cartegni, L. et al. ESEfinder: a web resource to identify exonic splicing enhancers. Nucleic Acid Research, 2003, 31(13): 3568-3571) and/or other available resources.

In one embodiment, the alternative exon is a naturally-occurring exon. In another embodiment, the alternative exon is derived from all or part of a known exon. In this context, “derived” refers to the alternative exon containing sequence that is substantially homologous to a naturally occurring exon, or a portion thereof, but may contain various mutations, such a mutations generated by altering exon splice enhancer (ESE) sequences and exon splice suppressor (ESS) sequences and/or by introducing ESE or ESS sequences into the alternative exon. “Homology” and “homologous” as used herein refer to the percent of identity between two polynucleotide sequences or between two polypeptide sequences. The correspondence between one sequence to another can be determined by techniques known in the art. For example, homology can be determined by a direct comparison of two polypeptide molecules by aligning their sequences and using readily available computer programs. Alternatively, homology can be determined by hybridization of polynucleotides under conditions which form stable duplexes between homologous regions, followed by digestion with single-stranded-specific nuclease(s), and size determination of the digested fragments. Two polynucleotide or two polypeptide sequences are “substantially homologous” to each other when, after optimally aligned with appropriate insertions or deletions, at least about 80%, at least about 85%, at least about 90%, and at least about 95% of the nucleotides or amino acids, respectively, match over a defined length of the molecules, as determined using the methods above.

In one embodiment, the alternative exon is exogenous to the sequence encoding a polypeptide disclosed herein, although it may be derived from a sequence originating from the organism where the sequence encoding a polypeptide disclosed herein will be expressed. As used herein, “exogenous” means derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). In one embodiment, the alternatively-spliced exon is derived from exon 2 of the human dihydrofolate reductase gene (DHFR), mutant human Wilms tumor 1 exon 5, mouse calcium/calmodulin-dependent protein kinase II delta exon 16, or SIRT1 exon 6. In embodiments, the alternatively-spliced exon is, or comprises, the modified DHFR exon 2 in SEQ ID NO:103 (GAATGAATTCAGATATTTCCAGAGAATGAAAAAAAAATCTTCAGTAGAAG). In embodiments, the alternatively-spliced exon is, or comprises, the modified DHFR exon 2 in SEQ ID NO:104 (GAATGAATTCAGATATTTCCAGAGAATGAAAAAAAATCTTCAGTAGAAG).

### Aptamer-Mediated Cleavage by Self-Cleaving Ribozymes

In one embodiment, the aptamer-mediated expression of the sequence encoding a polypeptide disclosed herein is regulated by an aptamer-mediated modulation of small endonucleolytic ribozymes. A ribozyme is an RNA enzyme that catalyzes a chemical reaction. In the nucleic acids and methods disclosed herein, a ribozyme may be any small endonucleolytic ribozyme that will self-cleave in the target cell type including, but not limited to a hammerhead, hairpin, the hepatitis delta virus, the Varkud satellite, twister, twister sister, pistol or hatchet ribozyme. Accordingly, in one embodiment, provided is a riboswitch, and a gene expression cassette comprising the riboswitch that contains a ribozyme linked to an aptamer. WO2017/136608, which is incorporated in its entirety by reference herein, describes such riboswitches that activate ribozyme self-cleavage in the presence of aptamer ligand (“off” switch) or riboswitches that inhibit ribozyme self-cleavage in the presence of aptamer (“on” switch).

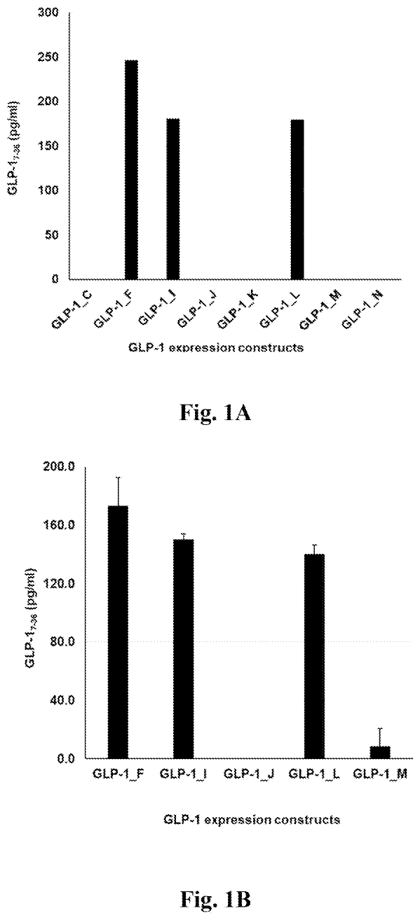

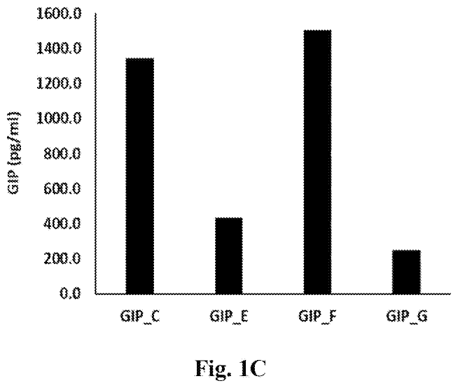

In an “off” switch scenario, aptamer/ligand binding increases the ribonuclease function of the ribozyme, leading to cleavage of the RNA of the sequence encoding a polypeptide disclosed herein that contains the polynucleotide cassette, thereby reducing expression of the sequence encoding a polypeptide disclosed herein. Examples of such an off switch include a polynucleotide cassette for the regulation of the expression of a sequence encoding a polypeptide disclosed herein comprising a riboswitch that comprises a twister ribozyme linked by a stem to an aptamer, wherein the stem linking the twister ribozyme to the aptamer attaches to the ribozyme at the location of the P3 stem of the twister ribozyme and wherein the sequence encoding a polypeptide disclosed herein is linked to the P1 stem of the twister ribozyme (see, e.g. FIGS. 1a, 1b, or 3a of WO2017/136608 and the associated text, incorporated herein by reference).

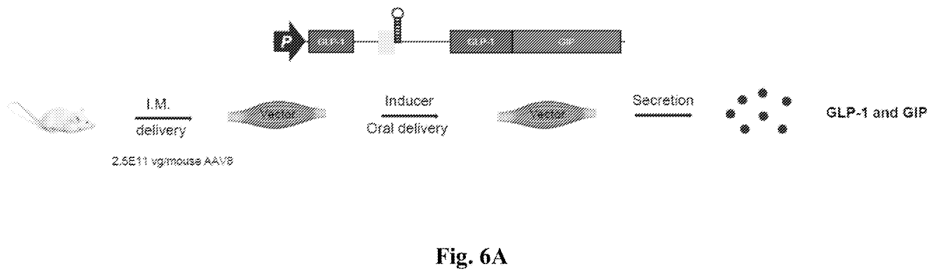

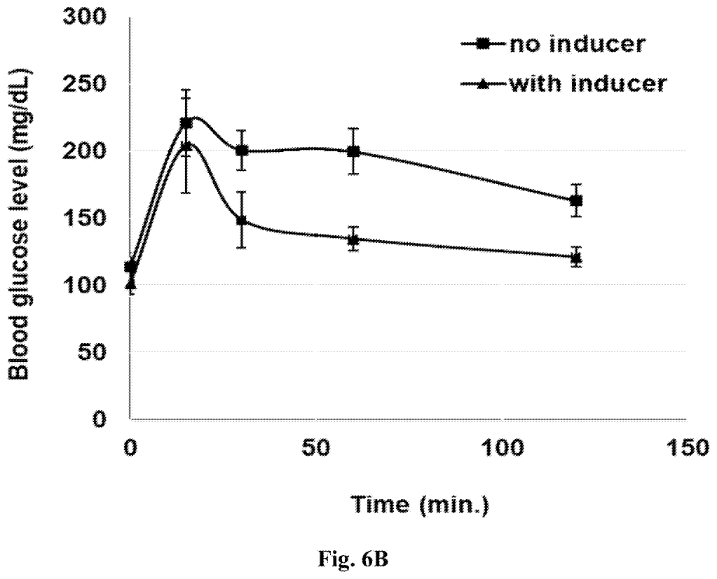

In an “on” switch scenario, aptamer/ligand binding inhibits the ribonuclease function of the ribozyme, decreasing cleavage of the RNA of the sequence encoding a polypeptide disclosed herein that contains the polynucleotide cassette, thereby increasing expression of the sequence encoding a polypeptide disclosed herein in the presence of ligand. Examples of an on switch include a riboswitch that comprises a twister ribozyme linked to an aptamer, wherein the aptamer is linked to the 3′ or 5′ end of the twister ribozyme P1 stem, wherein when the aptamer is linked to the 3′ end of the twister ribozyme P1 stem, a portion of the 3′ arm of the twister ribozyme P1 stem is alternatively the 5′ arm of the aptamer P1 stem, and wherein when the aptamer is linked to the 5′ end of the twister ribozyme P1 stem, a portion of the 5′ arm of the twister ribozyme P1 stem is alternatively the 3′ arm of the aptamer P1 stem (see, e.g., FIGS. 6a-6b of WO2017/136608 and the associated text, incorporated herein by reference).

### Aptamer Modulation of Polyadenylation

In embodiments, the expression of a sequence encoding a polypeptide disclosed herein is regulated by aptamer-modulated polyadenylation. The 3′ end of almost all eukaryotic mRNAs comprises a poly(A) tail—a homopolymer of 20 to 250 adenosine residues. Because addition of the poly(A) tail to mRNA protects it from degradation, expression of a gene can be influenced by modulating the polyadenylation the corresponding mRNA.

In one embodiment, the expression of the sequence encoding a polypeptide disclosed herein is regulated through aptamer-modulated accessibility of polyadenylation signals as described in and WO2018/156658, which is incorporated in its entirety by reference herein. In such embodiments, the riboswitch comprises an effector stem-loop and an aptamer, wherein the effector stem-loop comprises a polyadenylation signal, and wherein the aptamer and effector stem-loop are linked by an alternatively shared stem arm comprising a sequence that is complementary to the unshared arm of the aptamer stem and to the unshared arm of the effector stem loop (see, e.g., FIGS. 1a, 1b, 2a, and 5a of WO2018/156658 and the associated text, incorporated herein by reference). In one embodiment, the effector stem-loop is positioned 3′ of the aptamer such that the alternatively shared stem arm comprises all or a portion of the 3′ aptamer stem arm and all or a portion of the 5′ arm of the effector stem. In one embodiment, the effector stem-loop is positioned 5′ of the aptamer such that the alternatively shared stem arm comprises all or a portion of the 5′ aptamer stem arm and all or a portion of the 3′ arm of the effector stem. In one embodiment, the polyadenylation signal is AATAAA (SEQ ID NO:105) or ATTAAA (SEQ ID NO:106). In one embodiment, the polyadenylation signal is a downstream element (DSE). In one embodiment, the polyadenylation signal is an upstream sequence element (USE). In one embodiment, the polynucleotide cassette comprises two riboswitches, wherein the effector stem loop of the first riboswitch comprises all or part of the polyadenylation signal AATAAA (SEQ ID NO:105) or ATTAAA (SEQ ID NO:106) and the effector stem loop of the second riboswitch comprises all or part of the downstream element (DSE). In one embodiment, the two riboswitches each comprise aptamers that bind the same ligand. In one embodiment, the two riboswitches comprise different aptamers that bind different ligands.

In some embodiments, the riboswitch comprises a sensing region (e.g., an aptamer) and an effector region comprising a binding site for the small nuclear ribonucleoprotein (snRNP) U1, which is part of the spliceosome. WO2017/136591 describes riboswitches wherein the effector region comprises a U1 snRNP binding site, and is incorporated herein by reference in its entirety. When the aptamer binds its ligand, the effector region forms a stem and sequesters the U1 snRNP binding site from binding a U1 snRNP. Under certain conditions (for example, when the aptamer is not bound to its ligand), the effector region is in a context that provides access to the U1 snRNP binding site, allowing U1 snRNP to bind the mRNA and inhibit polyadenylation leading to degradation of the message. The U1 snRNP binding site can be any polynucleotide sequence that is capable of binding the U1 snRNP, thereby recruiting the U1 snRNP to the 3′ UTR of a sequence encoding a polypeptide disclosed herein and suppressing polyadenylation of the mRNA of the sequence encoding a polypeptide disclosed herein. In one embodiment, the U1 snRNP binding site is the consensus site CAGGTAAGTA (SEQ ID NO:107) (CAGGUAAGUA, SEQ ID NO:108, when in the mRNA). In some embodiments, the U1 snRNP binding site is a variation of this consensus sequence, including for example sequences that are shorter or have one or more nucleotides changed from the consensus sequence. In one embodiment, the U1 snRNP binding site contains the sequence CAGGTAAG (SEQ ID NO:109). In some embodiments, the binding site is encoded by the sequence selected from CAGGTAAGTA (SEQ ID NO:107), CAGGTAAGT (SEQ ID NO:110), and CAGGTAAG (SEQ ID NO:109). The U1 snRNP binding site can be any 5′ splice site from a gene, e.g., the 5′ splice site from human DHFR exon 2.

### Aptamer-Mediated Modulation of Ribonuclease Cleavage

In one embodiment, the expression of the sequence encoding a polypeptide disclosed herein is regulated through aptamer-modulated ribonuclease cleavage. Ribonucleases (RNases) recognize and cleave specific ribonuclease substrate sequences. Provided herein are recombinant DNA constructs that, when incorporated into the DNA of a sequence encoding a polypeptide disclosed herein, provide the ability to regulate expression of the sequence encoding a polypeptide disclosed herein by aptamer/ligand mediated ribonuclease cleavage of the resulting RNA. In some embodiments, the aptamer encoding sequence described herein is part of a construct that contains or encodes a ribonuclease substrate sequence and a riboswitch comprising an effector region and the aptamer such that when the aptamer binds a ligand, expression of the sequence encoding a polypeptide disclosed herein occurs (as described in WO2018/161053, which is incorporated in its entirety by reference herein). In embodiments, an RNase P substrate sequence is linked to a riboswitch wherein the riboswitch comprises an effector region and an aptamer, wherein the effector region comprises a sequence complimentary to a portion of the RNase P substrate sequence. Binding of a suitable ligand to the aptamer induces structural changes in the aptamer and effector region, altering the accessibility of the ribonuclease substrate sequence for cleavage by the ribonuclease.

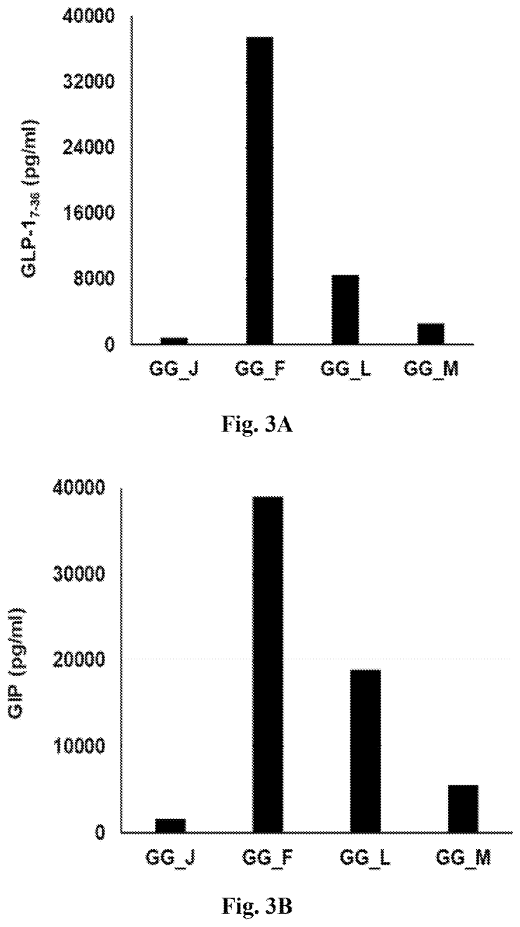

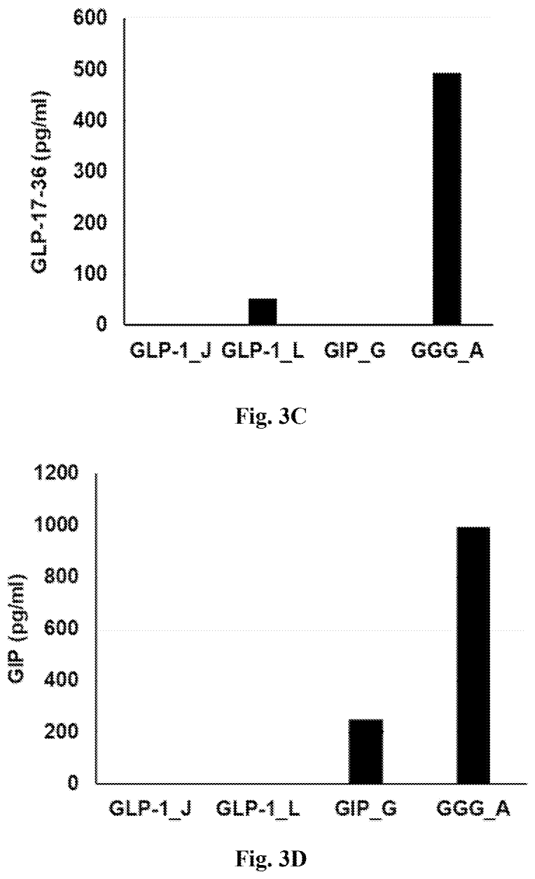

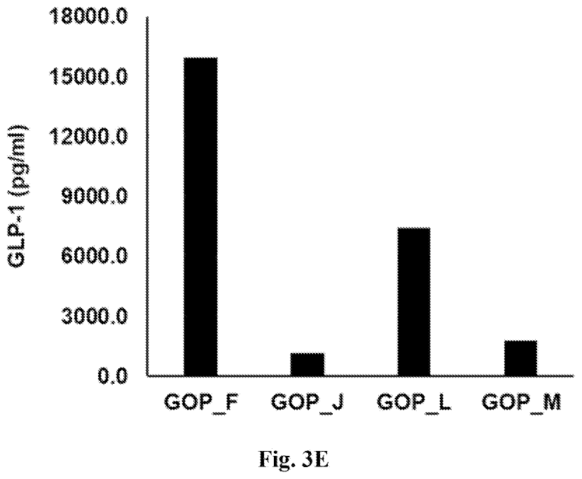

In one embodiment, the aptamer sequence is located 5′ to the RNase P substrate sequence and the effector region comprises all or part of the leader sequence and all or part of the 5′ acceptor stem sequence of the RNase P substrate sequence. See, e.g., FIGS. 1a, 1b, and 3b of WO2018/161053 and the associated text, incorporated herein by reference. In further embodiments, the acceptor stem of the RNase P substrate and the riboswitch effector region are separated by 0, 1, 2, 3, or 4 nucleotides. In other embodiments, the effector region stem includes, in addition to leader sequence (and its complement), one or more nucleotides of the acceptor stem of the RNase P substrate, and sequence complementary to the one or more nucleotides of the acceptor stem.

In one embodiment, the aptamer sequence of the polynucleotide cassette is located 3′ to the RNase P substrate sequence and the effector region comprises sequence complimentary to the all or part of the 3′ acceptor stem of the RNase P substrate sequence. See, e.g., FIG. 3a of WO2018/161053 and the associated text, incorporated herein by reference. In further embodiments, the effector region sequence complimentary to the 3′ acceptor stem of the RNase P substrate is 1 to 7 nucleotides. In other words, the effector region stem includes 1 to 7 nucleotides of the acceptor stem and includes sequence that is complementary to this 1 to 7 nucleotides of the acceptor stem. In embodiments, the riboswitch is located 3′ of the RNase P substrate, so the effector region stem and the acceptor stem of the RNase P substrate do not overlap. In embodiments, the effector region and the acceptor stem of the RNase P substrate are immediately adjacent (i.e., not overlapping). In other embodiments, the effector region and the acceptor stem of the RNase P substrate are separated by 1, 2, 3, 4, 5 or more nucleotides.

### Vectors

In one aspect, provided are recombinant vectors and their use for the introduction of a expression construct disclosed herein. In some embodiments, the vectors disclosed herein include additional DNA elements including DNA segments that provide for the replication of the DNA in a host cell and/or expression of a sequence encoding a polypeptide disclosed herein in target cells at appropriate levels. The ordinarily skilled artisan appreciates that expression control sequences (promoters, enhancers, and the like) are selected based on their ability to promote expression of the sequence encoding a polypeptide disclosed herein in the target cell. “Vector” means a recombinant plasmid, yeast artificial chromosome (YAC), mini chromosome, DNA mini-circle or virus (including virus derived sequences) that comprises a polynucleotide to be delivered into a host cell, either in vitro or in vivo. In one embodiment, the recombinant vector is a viral vector or a combination of multiple viral vectors.

Viral vectors for the expression of a sequence encoding a polypeptide disclosed herein in a target cell, tissue, or organism are known in the art and include adenoviral (AV) vectors, adeno-associated virus (AAV) vectors, retroviral and lentiviral vectors, and Herpes simplex type 1 (HSV1) vectors. Also included are viral particles comprising a nucleic acid encoding a polypeptide disclosed herein. In embodiments the viral particle as an AAV particle.

Adenoviral vectors include, for example, those based on human adenovirus type 2 and human adenovirus type 5 that have been made replication defective through deletions in the E1 and E3 regions. The transcriptional cassette can be inserted into the E1 region, yielding a recombinant E1/E3-deleted AV vector. Adenoviral vectors also include helper-dependent high-capacity adenoviral vectors (also known as high-capacity, “gutless” or “gutted” vectors), which do not contain viral coding sequences. These vectors contain the cis-acting elements needed for viral DNA replication and packaging, mainly the inverted terminal repeat sequences (ITR) and the packaging signal (CY). These helper-dependent AV vector genomes have the potential to carry from a few hundred base pairs up to approximately 36 kb of foreign DNA.

Recombinant adeno-associated virus “rAAV” vectors include any vector derived from any adeno-associated virus serotype, including, without limitation, AAV-1, AAV-2, AAV-3, AAV-4, AAV-5, AAV-7 and AAV-8, AAV-9, AAV-10, AAVrh10, and AAV2-retro (disclosed in PCT Patent Publication WO2017218842A1, which is incorporated herein in its entirety) and the like. rAAV vectors can have one or more of the AAV wild-type genes deleted in whole or in part, preferably the Rep and/or Cap genes, but retain functional flanking ITR sequences. Functional ITR sequences are retained for the rescue, replication, packaging and potential chromosomal integration of the AAV genome. The ITRs need not be the wild-type nucleotide sequences, and may be altered (e.g., by the insertion, deletion or substitution of nucleotides) so long as the sequences provide for functional rescue, replication and packaging.

Alternatively, other systems such as lentiviral vectors can be used. Lentiviral-based systems can transduce nondividing as well as dividing cells making them useful for applications targeting, for examples, the nondividing cells of the CNS. Lentiviral vectors are derived from the human immunodeficiency virus and, like that virus, integrate into the host genome providing the potential for very long-term gene expression.

Polynucleotides, including plasmids, YACs, minichromosomes and minicircles, carrying the sequence encoding a polypeptide disclosed herein containing the gene regulation cassette can also be introduced into a cell or organism by nonviral vector systems using, for example, cationic lipids, polymers, or both as carriers. Conjugated poly-L-lysine (PLL) polymer and polyethylenimine (PEI) polymer systems can also be used to deliver the vector to cells. Other methods for delivering the vector to cells includes hydrodynamic injection and electroporation and use of ultrasound, both for cell culture and for organisms. For a review of viral and non-viral delivery systems for gene delivery see Nayerossadat, N. et al. (Adv Biomed Res. 2012; 1:27) incorporated herein by reference.

### Pharmaceutical Compositions

Provided herein are pharmaceutical composition comprising any of the expression constructs, vectors, or viral particles disclosed herein and a pharmaceutically acceptable excipient. These compositions may comprise, in addition to the expression construct, vector, or viral particle, a pharmaceutically and/or physiologically acceptable excipient, carrier, buffer, stabilizer, antioxidants, preservative, or other additives well known to those skilled in the art. Such materials should be non-toxic and should not interfere with the efficacy of the active ingredient. The precise nature of the carrier or other material may be determined by the skilled person according to the route of administration. The pharmaceutical composition is typically in liquid form. Liquid pharmaceutical compositions generally include a liquid carrier such as water, petroleum, animal or vegetable oils, mineral oil or synthetic oil. Additional carriers are provided in International Patent Publication No. WO 00/15822, incorporated herein by reference. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used. In some cases, Ringer's Injection, Lactated Ringer's Injection, or Hartmann's solution is used. Preservatives, stabilizers, buffers, antioxidants and/or other additives may be included, as required.

For delayed release, the expression construct, vector, or viral particle may be included in a pharmaceutical composition which is formulated for slow release, such as in microcapsules formed from biocompatible polymers or in liposomal carrier systems according to methods known in the art.

If the expression construct, vector, or viral particle is to be stored long-term, it may be frozen in the presence of glycerol, or other cryopreservative.

### Methods

In one aspect, provided is a method of inducing satiation in a subject in need thereof, the method comprising administering to the subject an expression construct, a vector, or a pharmaceutical composition disclosed herein.

In one aspect, provided is a method of treating obesity in a subject in need thereof, the method comprising administering to the subject an expression construct, a vector, or a pharmaceutical composition disclosed herein.

In one aspect, provided is a method of suppressing appetite in a subject in need thereof, the method comprising administering to the subject an expression construct, a vector, or a pharmaceutical composition disclosed herein.

In one aspect, provided is a method of reducing weight gain in a subject in need thereof, the method comprising administering to the subject an expression construct, a vector, or a pharmaceutical composition disclosed herein.

In one aspect, provided is a method of improving glucose tolerance in a subject in need thereof, the method comprising administering to the subject an expression construct, a vector, or a pharmaceutical composition disclosed herein.

In one aspect, provided is a method of treating diabetes in a subject in need thereof, the method comprising administering to the subject an expression construct, a vector, or a pharmaceutical composition disclosed herein.

In one aspect, provided is a method of inducing insulin release in a subject in need thereof, the method comprising administering to the subject an expression construct, a vector, or a pharmaceutical composition disclosed herein.

Provided herein is an expression construct, vector, or pharmaceutical composition disclosed herein for use in a method of inducing satiation in a subject in need thereof.

Provided herein is an expression construct, vector, or pharmaceutical composition disclosed herein for use in a method of treating obesity in a subject in need thereof.

Provided herein is an expression construct, vector, or pharmaceutical composition disclosed herein for use in a method of suppressing appetite in a subject in need thereof.

Provided herein is an expression construct, vector, or pharmaceutical composition disclosed herein for use in a method of reducing weight gain in a subject in need thereof.

Provided herein is an expression construct, vector, or pharmaceutical composition disclosed herein for use in a method of improving glucose tolerance in a subject in need thereof.

Provided herein is an expression construct, vector, or pharmaceutical composition disclosed herein for use in a method of treating diabetes in a subject in need thereof.

Provided herein is an expression construct, vector, or pharmaceutical composition disclosed herein for use in is a method of inducing insulin release in a subject in need thereof.

Provided herein is the use of an expression construct, vector, or pharmaceutical composition disclosed herein in the manufacture of a medicament for inducing satiation in a subject in need thereof.

Provided herein is the use of an expression construct, vector, or pharmaceutical composition disclosed herein in the manufacture of a medicament for treating obesity in a subject in need thereof.

Provided herein is the use of an expression construct, vector, or pharmaceutical composition disclosed herein in the manufacture of a medicament for suppressing appetite in a subject in need thereof.

Provided herein is the use of an expression construct, vector, or pharmaceutical composition disclosed herein in the manufacture of a medicament for reducing weight gain in a subject in need thereof.

Provided herein is the use of an expression construct, vector, or pharmaceutical composition disclosed herein in the manufacture of a medicament for improving glucose tolerance in a subject in need thereof.

Provided herein is the use of an expression construct, vector, or pharmaceutical composition disclosed herein in the manufacture of a medicament for treating diabetes in a subject in need thereof.

Provided herein is the use of an expression construct, vector, or pharmaceutical composition disclosed herein in the manufacture of a medicament for inducing insulin release in a subject in need thereof.

Provided herein is a method of treating a subject in in need of increased expression of a polypeptide (including a polyprotein) disclosed herein encoded by a sequence encoding the polypeptide, the method comprising administering to the patient a pharmaceutical composition comprising a ligand, which an aptamer binds to or otherwise responds to, wherein the patient previously had been administered a recombinant DNA comprising the sequence encoding the polypeptide, and where the sequence encoding the polypeptide contains a gene regulation cassette disclosed herein that provides the ability to regulate expression of the target gene by the ligand of the aptamer.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), or enhancement or improvement of the condition, disorder or disease. Treatment includes eliciting a clinically significant response without excessive levels of side effects.

In one aspect, in the methods disclosed herein, the expression construct is delivered by gene therapy. The cell specificity of the sequence encoding a polypeptide disclosed herein may be controlled by a promoter and/or other elements within the vector and/or by the capsid of the viral vector. Delivery of the vector construct containing the sequence encoding a polypeptide disclosed herein, and the transfection of the target tissues resulting in stable transfection of the regulated sequence encoding a polypeptide disclosed herein, is the first step in producing the polypeptide.

In some embodiment, if an aptamer within the sequence encoding the polypeptide disclosed herein is used, the sequence encoding the polypeptide disclosed herein is not expressed at significant levels, i.e., it is in the “off state” in the absence of the specific ligand that binds to the aptamer contained within in the regulatory cassette riboswitch. Only when the aptamer specific ligand is administered is the expression of the sequence encoding the polypeptide disclosed activated. The delivery of the vector construct containing the sequence encoding the polypeptide disclosed herein and the delivery of the activating ligand generally are separated in time. The delivery of the activating ligand will control when the sequence encoding the polypeptide disclosed herein is expressed, as well as the level of protein expression.

The expression construct, vector, or pharmaceutical composition disclosed herein (and the ligand in case of aptamer-mediated regulation of gene expression) may be delivered by a number of routes including, but not limited to, intravitreal, intraocular, inhalation, subcutaneous, intramuscular, intradermal, intralesion, topical, intraperitoneal, intravenous (IV), intra-arterial, perivascular, intracerebral, intracerebroventricular, oral, sublingual, sublabial, buccal, nasal, intrathoracic, intracardiac, intrathecal, epidural, intraosseous, or intraarticular.

If an aptamer is used, the timing of delivery of the ligand can be adjusted as needed. For example, an oral small molecule ligand may be delivered daily, or multiple times a day. Alternatively, the inducing ligand may be dosed less frequently, for example, once a week, every other week, once a month.

### Articles of Manufacture and Kits

Also provided are kits or articles of manufacture for use in the methods described herein. In aspects, the kits comprise the compositions described herein (e.g., compositions for delivery of a vector comprising an expression construct disclosed herein) in suitable packaging. Suitable packaging for compositions (such as ocular compositions for injection) described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

Also provided are kits comprising the compositions described herein. These kits may further comprise instruction(s) on methods of using the composition, such as uses described herein. The kits described herein may further include other materials desirable from a commercial and user standpoint, including buffers, diluents, filters, needles, syringes, and package inserts with instructions for performing the administration of the composition or performing any methods described herein. For example, in some embodiments, the kit comprises an rAAV for the expression of polypeptide disclosed herein, a pharmaceutically acceptable carrier suitable for injection, and one or more of: a buffer, a diluent, a filter, a needle, a syringe, and a package insert with instructions for performing the injections. In some embodiments, the kit is suitable for intraocular injection, intramuscular injection, intravenous injection and the like.

It is to be understood and expected that variations of the compositions of matter and methods herein disclosed can be made by one skilled in the art and it is intended that such modifications are to be included within the scope of the present disclosure. The following Examples further illustrate the invention, but should not be construed to limit the scope of the invention in any way.

All references cited herein are hereby incorporated by reference in their entirety. All nucleotide sequences provided herein are in a 5′ to 3′ orientation unless stated otherwise. A Sequence Listing is filed herewith, the contents of which are incorporated herein by reference in its entirety.

### Overview of Sequences Disclosed Herein

### Selected Gut Peptides (See Also Tables 1 and 2)

| | SEQ ID NO | SEQ ID NO |
|---|---|---|
| Gut peptide | Amino acid | Nucleic acid sequence |
| hGLP-1 | 1 | 6, 7, and 8 |
| hGIP | 2 | 9 |
| hOXM | 3 | 10 |
| PYY | 4 | 11 |
| hGlucagon | 5 | 12 |

### Selected Signal Peptides (See Also Tables 3 and 4)

| | SEQ ID NO | SEQ ID NO |
|---|---|---|
| Signal peptide | Amino acid | Nucleic acid sequence |
| IgM | 13 | 21 |
| hInsul | 14 | 22 |
| mIgh | 15 | 23 |
| hGH | 16 | 24 |
| mEpo | 17 | 25 |
| mGHRH | 18 | 26 |
| hAlbumin | 19 | 27 |
| hFIX | 20 | 28 |

### Exemplary Monocistronic Constructs (See Also Tables 5 and 6)

| Construct | Signal | Encoded gut | SEQ ID NO | SEQ ID NO |
|---|---|---|---|---|
| name | peptide | peptide | Amino acid | Nucleic acid |
| GLP-1_C | IgM SP | hGLP-1 | 29 | 37 |
| GLP-1_F | hInsul | hGLP-1 | 30 | 38 |
| GLP-1_I | mIgh | hGLP-1 | 31 | 39 |
| GLP-1_J | hGH | hGLP-1 | 32 | 40 |
| GLP-1_K | mEpo | hGLP-1 | 33 | 41 |
| GLP-1_L | mGHRH | hGLP-1 | 34 | 42 |
| GLP-1_M | hAlbumin | hGLP-1 | 35 | 43 |
| GLP-1_N | hFIX9 | hGLP-1 | 36 | 44 |
| GIP_C | IgM SP | hGIP | 111 | |
| GIP_E | hFIX9 | hGIP | 112 | |
| GIP_F | hAlbumin | hGIP | 113 | |
| GIP_G | hGH | hGIP | 114 | |

### Exemplary Bicistronic Constructs (See Also Tables 7 and 8)

| | | | SEQ | SEQ |
|---|---|---|---|---|
| | | | ID NO | ID NO |
| | Signal | | Amino | Nucleic |
| Construct name | peptide | Gut peptides | acid | acid |
| 2xGLP-1_2xB | hAlbumin | hGLP-1, hGLP-1 | 45 | 50 |
| GG_J | hGH | hGLP-1, hGIP | 46 | 51 |
| GG_F | hInsul | hGLP-1, hGIP | 47 | 52 |
| GG_L | mGHRH | hGLP-1, hGIP | 48 | 53 |
| GG_M | hAlbumin | hGLP-1, hGIP | 49 | 54 |
| 2xGLP-1 w/o signal | n/a | hGLP-1, hGLP-1 | 55 | 57 |
| peptide | | | | |
| GG w/o signal peptide | n/a | hGLP-1, hGIP | 56 | 58 |

### Exemplary Tricistronic Constructs (See Also Tables 9 and 10)

| | Signal | | SEQ ID NO | SEQ ID NO |
|---|---|---|---|---|
| Construct name | peptide | Gut peptides | Amino acid | Nucleic acid |
| 3xGLP-1_3xB | hAlbumin | hGLP-1, hGLP-1, hGLP-1, | 59 | 67 |
| 3xGLP-1_3xC | hInsul | hGLP-1, hGLP-1, hGLP-1, | 60 | 68 |
| 3xGLP-1_3xD | mGHRH | hGLP-1, hGLP-1, hGLP-1, | 61 | 69 |
| GOP_J | hGH | hGLP-1, hOXM, PYY | 62 | 70 |
| GOP_F | hInsul | hGLP-1, hOXM, PYY | 63 | 71 |
| GOP_L | mGHRH | hGLP-1, hOXM, PYY | 64 | 72 |
| GOP_M | hAlbumin | hGLP-1, hOXM, PYY | 65 | 73 |
| GGG_A | hGH | hGLP-1, hGlucagon, hGIP | 66 | 74 |
| 3xGLP-1 w/o signal | n/a | hGLP-1, hGLP-1, hGLP-1, | 75 | 78 |
| peptide | | | | |
| hGLP-1, hOXM, PYY | n/a | hGLP-1, hOXM, PYY | 76 | 79 |
| w/o signal peptide | | | | |
| hGLP-1, hGlucagon, | n/a | hGLP-1, hGlucagon, hGIP | 77 | 80 |
| hGIP w/o signal peptide | | | | |

Other sequences

| SEQ | |
|---|---|
| ID NO | Sequence |
| 81 | TACCCATACGATGTTCCAGATTACGCTAGAAAAAAGAGA |
| 84 | TTCCCAACCATTCCCTTATCCAGGCTTTTTGACAACGCTATGCTCCGCGCCAGAAAA |
| | AAGAGA |
| 85 | GCTCCCCCACGCCTCATCTGCGACAGTCGAGTTCTGGAGAGGTACAGAAAAAAGAGA |
| 86 | CTGCCCCCCTCACCTCCCTTCAGGATGCAGCGA |
| 87 | AGGGGTGTGTTTCGTCGA |
| 88 | ACAGTTTTTCTTGATCATGAAAACGCCAACAAAATTCTGAATCGGCCAAAGAGG |
| 89 | YPYDVPDYARKKR |
| 92 | FPTIPLSRLFDNAMLRARKKR |
| 93 | APPRLICDSRVLERYRKKR |
| 94 | LPPSPPFRMQR |
| 95 | RGVFRR |
| 96 | TVFLDHENANKILNRPKR |
| 97 | RKKR |
| 98 | RMQR |
| 99 | VERR |
| 100 | RPKR |
| 101- | GTGAGTCTATGGGACCCTTGATGTTTTCTTTCCCCTTCTTTTCTATGGTTAAGTTCA |
| X-106 | TGTCATAGGAAGGGGAGAAGTAACAGGGTACACATATTGACCAAATCAGGGTAATTT |
| | TGCATTTGTAATTTTAAAAAATGCTTTCTTCTTTTAATATACTTTTTTGTTTATCTT |
| | ATTTCTAATACTTTCCCTAATCTCTTTCTTTCAGGGCAATAATGATACAATGTATCA |
| | TGCCGAGTAACGCTGTTTCTCTAACTTGTAGGAATGAATTCAGATATTTCCAGAGAA |
| | TGAAAAAAAAATCTTCAGTAGAAGgtaatgt-X- |
| | acattacGCACCATTCTAAAGAATAACAGTGATAATTTCTGGGTTAAGGCAATAGCA |
| | ATATTTCTGCATATAAATATTTCTGCATATAAATTGTAACTGATGTAAGAGGTTTCA |
| | TATTGCTAATAGCAGCTACAATCCAGCTACCATTCTGCTTTTATTTTATGGTTGGGA |
| | TAAGGCTGGATTATTCTGAGTCCAAGCTAGGCCCTTTTGCTAATCATGTTCATACCT |
| | CTTATCTTCCTCCCACAG |
| 101 | GTGAGTCTATGGGACCCTTGATGTTTTCTTTCCCCTTCTTTTCTATGGTTAAGTTCA |
| | TGTCATAGGAAGGGGAGAAGTAACAGGGTACACATATTGACCAAATCAGGGTAATTT |
| | TGCATTTGTAATTTTAAAAAATGCTTTCTTCTTTTAATATACTTTTTTGTTTATCTT |
| | ATTTCTAATACTTTCCCTAATCTCTTTCTTTCAGGGCAATAATGATACAATGTATCA |
| | TGCCGAGTAACGCTGTTTCTCTAACTTGTAGGAATGAATTCAGATATTTCCAGAGAA |
| | TGAAAAAAAAATCTTCAGTAGAAGgtaatgt |
| 116 | ACATTACGCACCATTCTAAAGAATAACAGTGATAATTTCTGGGTTAAGGCAATAGCA |
| | ATATTTCTGCATATAAATATTTCTGCATATAAATTGTAACTGATGTAAGAGGTTTCA |
| | TATTGCTAATAGCAGCTACAATCCAGCTACCATTCTGCTTTTATTTTATGGTTGGGA |
| | TAAGGCTGGATTATTCTGAGTCCAAGCTAGGCCCTTTTGCTAATCATGTTCATACCT |
| | CTTATCTTCCTCCCACAG |
| 102 | X₁AGGTX₂AGT |
| | X₁ = A or C |
| | X₂ = A or G |
| 103 | GAATGAATTCAGATATTTCCAGAGAATGAAAAAAAAATCTTCAGTAGAAG |
| 104 | GAATGAATTCAGATATTTCCAGAGAATGAAAAAAAATCTTCAGTAGAAG |
| 105 | AATAAA |
| 106 | ATTAAA |
| 107 | CAGGTAAGTA |
| 108 | CAGGUAAGUA |
| 109 | CAGGTAAG |
| 110 | CAGGTAAGT |
| 115 | agaaagaagagaCATGCTGAAGGGACATTTACCTCAGATGTTTCTTCATACCTGGAA |
| | GGACAGGCTGCCAAGGAATTTATTGCATGGCTTGTGAAAGGCAGGGGCTGA |

### EXAMPLES

### Example 1: Expression of Secretable Gut Peptides Using Monocistronic Expression Constructs

### Experimental Procedures

For the construction of expression vector encoding secretable peptides, gene fragments containing DNA sequences encoding signal peptides, sequences facilitating furin cleavage sites, and human GLP-1₇₋₃₇ or hGIP were synthesized (IDT) and cloned into expression constructs containing a CMV promoter.

For the transfection, 3.5×10⁴ human embryonic kidney (HEK) 293 cells were plated in a 96-well flat bottom plate the day before transfection. Plasmid DNA (500 ng) was added to a tube or a 96-well U-bottom plate. Separately, TransIT-293 reagent (Mirus; 1.4 μL) was added to 50 μL Optimum I media (Life Technologies) and allowed to sit for 5 minutes at room temperature (RT). Then, 50 μL of this diluted transfection reagent was added to the DNA, mixed, and incubated at RT for 20 min. Finally, 7 μL of this solution was added to a well of cells in the 96-well plate. The supernatants of the transfected cell were collected 48 hours after transfection and assayed for GLP-1 or GIP peptide.

For the enzyme-linked immunosorbent assay (ELISA) for GLP-1, HEK 293 cells were transfected using TransIT-293 transfection reagent (Mirus Bio) with the constructs containing coding sequences for the active form of human GLP-1 peptide. 200 nM of Sitagliptin phosphate monohydrate (Sigma) was added to the culture for inhibiting dipeptidyl peptidase DPP-IV in the culture medium. The supernatants from the transfected cells were collected 48 hours after transfection and were subjected to SingleStep ELISA for the detection of human GLP-1₇₋₃₆ in the supernatant following the manufacturer's instruction (Abcam).

### Results:

To generate secretable GLP-1 peptides expression constructs, a sequence encoding a leader sequence (comprising of a signal peptide sequence and a furin recognition and cleavage sequence), was generated and fused to the 5′ end of sequence encoding the GLP-1₇₋₃₇ peptide. The signal peptide sequences from various secretory proteins were selected and tested for their ability to promote secretion of GLP-1. The furin recognition and cleavage sequences comprised sequences with the minimal furin cleavage site (RXXR for consensus furin cleavage site) and a sequence N-terminal of the cleavage site that facilitates furin recognition and cleavage. Inclusion of furin recognition and cleavage sequence in the leader sequence promoted the functional N-terminus of the GLP-1 peptide to be fully processed and generated in non-endocrine cells.

The expression of the active GLP-1 peptide was assayed using ELISA specific for active GLP-17-36. As shown in FIG. 1A, of all the eight constructs (see Tables 5 and 6) generated for expressing GLP-1, only constructs GLP-1_F (with human insulin signal peptide), _I (with mouse Ig heavy chain signal peptide) and _L (with mouse GHRH signal peptide) produced detectable active GLP-1 peptides.

A second validation experiment confirmed the GLP1-₇₋₃₇ expression from construct GLP-1_F, I and L (FIG. 1B). Construct GLP-1_M (with human albumin signal peptide) expressed very low but detectable amount of GLP-1. These expression results suggest that signal peptides from different secretory proteins have different efficiency in promoting secretable GLP-1 expression. In addition, as the ELISA assay detected the furin-cleaved peptide. As such, a low and undetectable expression of GLP-1₇₋₃₇ is likely due to the inefficient furin cleavage.

The same strategy was used to generate the constructs for the expression of secretable human glucose dependent insulinotropic peptide (GIP₁₋₄₂). The same leader sequences used in constructs GLP-1_C, N, M and J (see Tables 5 and 6) were used to construct GIP_C, E, F and G, respectively. Expression of the GIP peptides was assayed using an ELISA that detects total GIP (including both GIP₁₋₄₂ and GIP₃₋₄₂). GIP constructs containing the leader sequences that lead to very low or undetectable amount of GLP-1 secretion expressed significant amount of GIP (FIG. 1C). However, the assay used here recognized the C-terminus of the GIP, therefore the efficiency of furin cleavage (at the N-terminus of the peptide) was not reflected in the assay.

### Example 2: Expression of Secretable Gut Peptides Using Bi- and Tri-Cistronic Expression Constructs

To improve the expression level of gut peptides, bi- and tricistronic expression constructs were generated encoding polyproteins comprising two or three GLP-1 peptides. The peptides were separated by a minimal furin cleavage site sequence (RXXR). Posttranslational furin processing of the polyprotein in non-endocrine cells led to the release of individual peptides.

First, a GLP-1 expression vector was constructed that built on the GLP-1_M construct (encoding for a polypeptide comprising a human albumin signal peptide sequence and a sequence containing a furin cleavage site downstream of the signal peptide, see SEQ ID NO:35). The GLP-1 encoding sequence can be any polynucleotide sequence that encodes the GLP-1₇₋₃₇ peptide.

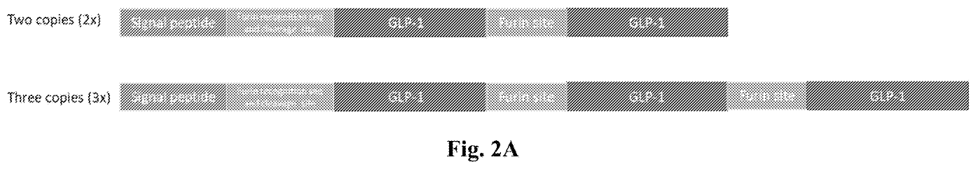

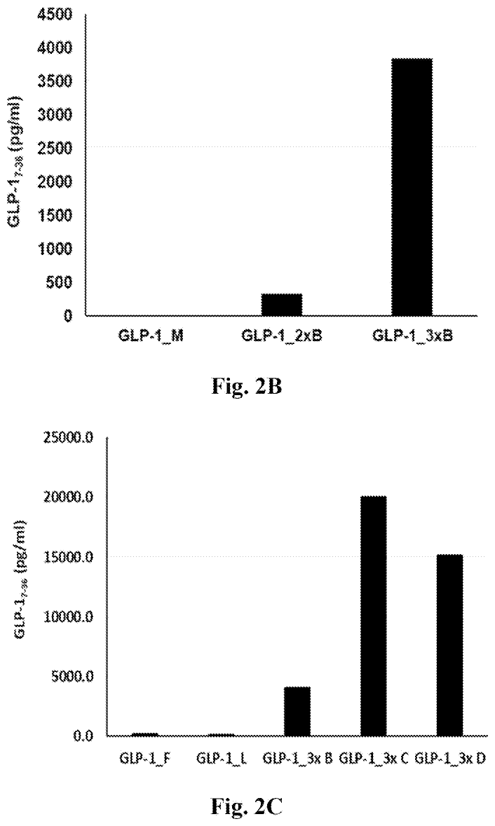

Since the GLP-1_M construct (see Tables 5 and 6) expresses very low amounts of GLP-1, it was tested whether two copies (bicistronic construct 2xGLP-1_2xB, see Tables 7 and 8) or three copies (tricistronic construct 3xGLP-1_3xB, see Tables 9 and 10) of the GLP-1 coding sequence would increase the peptide's expression level (FIG. 2A). As shown in FIG. 2B, two copies of the GLP-1 coding sequence indeed increased active GLP-1 expression, and three copies even substantially increased GLP-1 expression even further.

Next, tricistronic expression constructs 3xGLP-1_3xC and 3xGLP-1_3xD (see Tables 9 and 10) were generated (containing the leader sequences as in construct GLP-1_F and GLP-1_L, respectively, see Tables 5 and 6). As shown in FIG. 2C, the tricistronic construct expressed more than 100 times the amount of GLP-1 as compared to monocistronic constructs GL-1_F and GLP-1_L. As only furin-cleaved GLP-1 can be detected in the assay, this enhanced high level of GLP-1₇₋₃₇ expression is also an indication of efficient furin cleavage at the inserted furin sites that link each individual peptide and efficient posttranslational processing.

These results demonstrated that small peptide expression can be enhanced by using the expression constructs disclosed herein.

### Example 3: Bi- and Tri-Cistronic Expression Constructs for the Expression of Different Types of Gut Peptides

### Experimental Procedures

To construct the expression vectors encoding secretable multiple peptides, gene fragments containing DNA sequences encoding signal peptides, furin cleavage sequence and human GLP-1₇₋₃₇, human glucose dependent insulinotropic peptide (GIP₁₋₄₂) and human peptide tyrosine (PYY₃₋₃₆), and human oxyntomodulin (OXM) were synthesized (IDT) and cloned into expression constructs containing CMV promoter.

For the ELISA to detect human glucose-dependent insulinotropic polypeptide (GIP), HEK 293 cells were transfected with TransIT-293 transfection reagent (Mirus Bio) with the constructs containing coding sequences for secretable human GLP-1₇₋₃₇ and GIP₁₋₄₂ peptides or containing coding sequence for GLP-1₇₋₃₇, Oxyntomodulin and peptide tyrosine (PYY₃-36). 200 nM of Sitagliptin phosphate monohydrate (Sigma) was added to the culture for inhibiting dipeptidyl peptidase DPP-IV in the culture medium. The supernatants from the transfected cells were collected 48 hours after transfection and were subjected to ELISA assay for active GLP-17-36 (Abcam) and total GIP (EMD Millipore) and total PYY (EMD Millipore) following manufacturer's instruction.

### Results:

Next, expression constructs were designed encoding polyproteins comprising different gut peptides, using the strategy of generating polyprotein sequences discussed in Example 2.

The leader sequences used in constructs GLP-1_J, F, L and M (see Tables 5 and 6) were used to construct GG_J, F, L, M, respectively, for co-expressing the GLP-1₇₋₃₇ peptide and the GIP₁₋₄₂ peptide (GG) (see Tables 7 and 8).

As shown in FIG. 3A, all four bicistronic GG expression constructs expressed higher levels of the GLP-1₇₋₃₇ peptide as compared to monocistronic GLP-1 expression constructs. Constructs encoding for polypeptides with a human insulin signal sequence expressed the highest amount of GLP-1₇₋₃₇ peptide (see GG_F in FIG. 3A and GLP-1_F in FIGS. 1A and 1B).

Similarly, the GIP expression from GG constructs is higher than that from constructs with single of GIP, and GG_F expressed the highest amount of GIP (FIG. 3B).

Next, a tricistronic expression construct was generated encoding a polyprotein comprising the GLP-1₇₋₃₇ peptide, a glucagon peptide, and the GIP₁₋₄₂ peptide. Tricistronic construct GGG_A (see Tables 9 and 10) has the same leader sequence as monocistronic constructs GLP-1_J and GIP_G (see Tables 5 and 6), but shows significantly higher expression of both the GLP-1₇₋₃₇ peptide (FIG. 3C) and the GIP₁₋₄₂ peptide (FIG. 3D) compared to the monocistronic constructs.

Next, the leader sequences used in constructs GLP-1_J, F, L and M (see Tables 5 and 6) were used to construct GOP_J, F, L and M, respectively, (see Tables 9 and 10) for co-expressing the GLP-1₇₋₃₇ peptide, OXM peptide and PYY (GOP). As shown in FIG. 3E, all the four tricistronic GOP constructs expressed higher levels of the GLP-1 peptide as compared to the monocistronic constructs. Again, GOP_F expressed the highest amount of GLP-1₇₋₃₇ peptide. Further, the GOP_F construct expressed approximately 27,158 pg/ml of PYY₃₋₃₆.

These results demonstrate that tricistronic expression constructs disclosed herein provide for significantly increased expression as compared to monocistronic expression constructs and constitute an efficient strategy for co-expressing multiple small peptides.

### Example 4: Riboswitch-Regulated Expression of Gut Peptides

Next, it was investigated whether the controlled expression of gut peptides can be used in gene therapy for treating diabetes, obesity, and other metabolic indications. To that end, riboswitch N5-12G6 cassette (GTGAGTCTATGGGACCCTTGATGTTTTCTTTCCCCTTCTTTTCTATGGTTAAGTTCAT GTCATAGGAAGGGGAGAAGTAACAGGGTACACATATTGACCAAATCAGGGTAATTT TGCATTTGTAATTTTAAAAAATGCTTTCTTCTTTTAATATACTTTTTTGTTTATCTTAT TTCTAATACTTTCCCTAATCTCTTTCTTTCAGGGCAATAATGATACAATGTATCATGC CGAGTAACGCTGTTTCTCTAACTTGTAGGAATGAATTCAGATATTTCCAGAGAATGA AAAAAAAATCTTCAGTAGAAGGTAATGTCTGGGGAGTCCTTCATGCGGGGCTGAGA GGATGGAAGCAATCGACCATCGACCCATTGCACCTGATCCGGTATGTCCCGGCGCA GGGAGACATTACGCACCATTCTAAAGAATAACAGTGATAATTTCTGGGTTAAGGCA ATAGCAATATTTCTGCATATAAATATTTCTGCATATAAATTGTAACTGATGTAAGAG GTTTCATATTGCTAATAGCAGCTACAATCCAGCTACCATTCTGCTTTTATTTTATGGT TGGGATAAGGCTGGATTATTCTGAGTCCAAGCTAGGCCCTTTTGCTAATCATGTTCA TACCTCTTATCTTCCTCCCACAG, SEQ ID NO:117), was inserted into the encoding polypeptide sequence in bicistronic expression construct GG_L (expressing the hGLP-1₇₋₃₇ peptide and the hGIP₁₋₄₂ peptide, see Tables 7 and 8) between nucleotide 81 and 82, or between 101 and 102, or between 137 and 138, or between 151 and 152, or between 176 and 177, or between 206 and 207, or between 286 and 287 (count starting with the start codon), respectively, generating regulatable, riboswitch-containing bicistronic expression constructs GG_L_1 through 7.

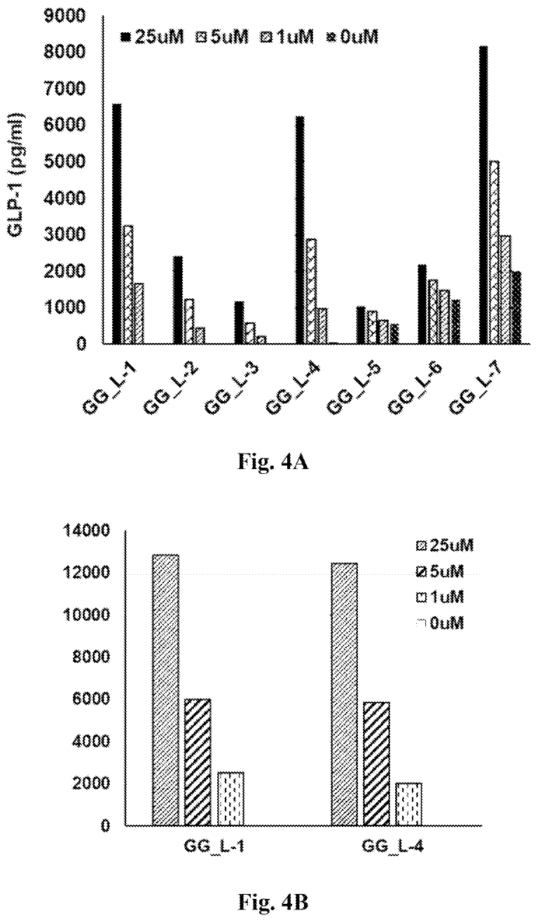

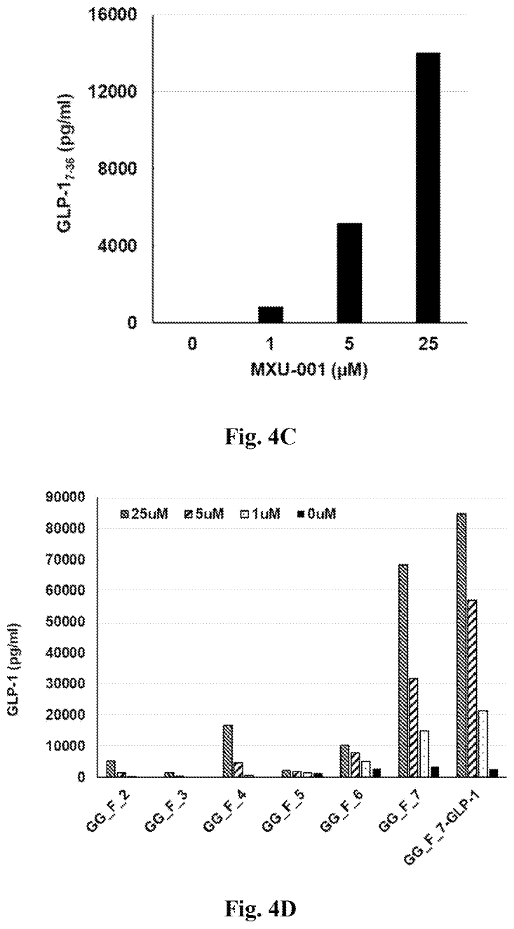

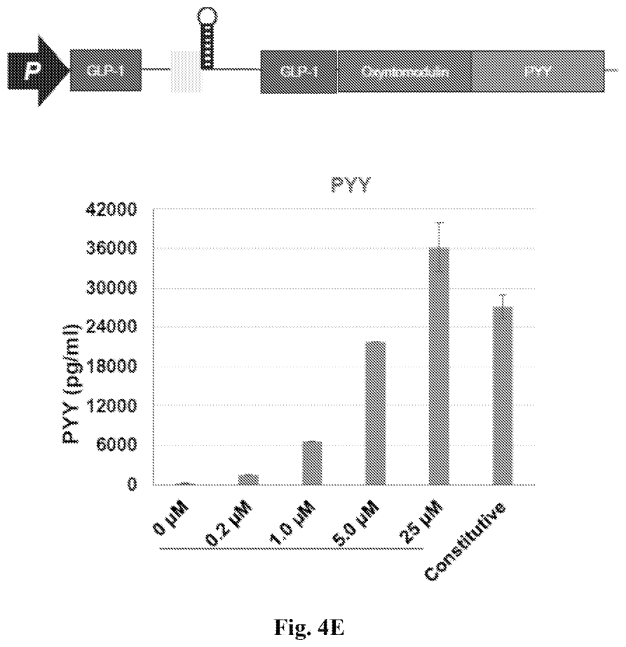

As shown in FIG. 4A, after transfection into HEK 293 cells, these constructs expressed the hGLP-1₇₋₃₇ peptide in response to the small molecule inducer treatment in a dose dependent manner. The regulated expression of the other peptide, hGIP₁₋₄₂, expressed from the same construct, was also determined. Constructs GG_1 and GG_L_4 expressed the hGIP₁₋₄₂ peptide in a dose dependent manner (FIG. 4B).

Next, the riboswitch cassette was inserted in construct tricistronic expression construct 3xGLP-1_3xC (see Tables 9 and 10) at nucleotide position (counts from start codon in the polypeptide encoding sequence) between position 172 and 173, resulting in the regulatable GLP-1_3xC_4 construct. As shown in FIG. 4C, the GLP-1_3xC_4 construct expressed the GLP-1₇₋₃₇ peptide in response to the small molecule inducer treatment in a dose dependent manner.

Next, the riboswitch cassette was inserted into the bicistronic expression construct GG_F (expressing the hGLP-1₇₋₃₇ peptide and the hGIP₁₋₄₂ peptide, see Tables 7 and 8) at nucleotide position (counts from the start codon) between nucleotide 122 and 123, or between 158 and 159, or between 172 and 173, or between 197 and 198, or between 227 and 228, or between 307 and 308, respectively, generating regulatable bicistronic constructs GG_F_2 through 7. As shown in FIG. 4D, after transfection into HEK 293 cells, these constructs expressed the GLP-1₇₋₃₇ peptide in response to the small molecule inducer treatment in a dose dependent manner. Particularly, regulatable, bicistronic expression construct GG_F_7 expressed the highest level of GLP-1₇₋₃₇ peptide at each indicated concentration of small molecule inducer.

Next, a sequence encoding an additional furin cleavage site and an additional copy of the GLP-1₇₋₃₇ peptide (agaaagaagagaCATGCTGAAGGGACATTTACCTCAGATGTTTCTTCATACCTGGAAGGA CAGGCTGCCAAGGAATTTATTGCATGGCTTGTGAAAGGCAGGGGCTGA, SEQ ID NO:115) was added 3′ of the nucleic acids encoding the last amino acid codon in the GG_F_7 construct, resulting in the regulatable, tricistronic GG_F_7-GLP-1 expression construct. This GG_F_GLP-1 construct (encoding a polyprotein comprising two copies of the hGLP-1₇₋₃₇ peptide and one copy of the hGIP₁₋₄₂ peptide) expressed further enhanced level of the hGLP-1₇₋₃₇ peptide in a dose responsive manner (FIG. 4D).

Finally, a regulatable tricistronic expression construct expressing a polyprotein comprising GLP-1, hOXM, and PYY expressed PYY in a dose dependent manner in response to the inducer (FIG. 4E).

### Example 5: GLP-1 and GIP Peptides are Biological Active In Vitro and In Vivo

### Experimental Procedure

For the transfection, 3.5×10⁴ human embryonic kidney (HEK) 293 cells were plated in a 96-well flat bottom plate the day before transfection. Plasmid DNA (500 ng) was added to a tube or a 96-well U-bottom plate. Separately, TransIT-293 reagent (Mirus; 1.4 μL) was added to 50 μL Optimum I media (Life Technologies) and allowed to sit for 5 minutes at room temperature (RT). Then, 50 μL of this diluted transfection reagent was added to the DNA, mixed, and incubated at RT for 20 min. Finally, 7 μL of this solution was added to a well of cells in the 96-well plate. The supernatants of the transfected cell were collected 48 hours after transfection and used as conditioned medium for the source of expressed GLP-1 or GIP peptide.

For the GLP-1 and GIP bioactivity assay, HEK 293 cells stably expressing the human GLP-1 receptor (HEK-293-hGLP-1R) or expressing the human GIP receptor (HEK-293-hGIPR) were generated by stably transfecting HEK293 cells with pCMV3 plasmid containing hGLP-1R cDNA or hGIPR cDNA (SinoBiological). The established stable cell lines were transfected with pCRE Tluc16-DD (Thermo Scientific) that contains TurboLuc luciferase gene driven under cAMP response element (CRE) promoter. 5 hours after transfection, the transfected cells were plated in 96-well plate at a 2×10⁴ cells per well the day before the addition of the conditioned medium containing GLP-1 and/or GIP peptides. 1 hour after the addition of 100 μl conditioned medium, a luciferase assay was performed using TurboLuc Luciferase One-step glow assay kit (Thermo Scientific) following manufacturer's instruction, and luminescence was measure using Tecan microplate reader.

For AAV2/8 (AAV2 genome, AAV8 capsid) viral particle production, expression construct GG_F encoding hGLP-1 and hGIP was cloned into an AAV2 plasmid vector. Expression of the hGLP-1 and hGIP genes was driven by CASI promoter, which includes CMV and ubiquitin C enhancer elements and the chicken j-actin promoter. The AAV plasmid vector was packaged into an AAV8 capsid, generating AAV viral vector AAV8.GG_F.

For the animal studies, male C57Bl/6 mice (Jackson Laboratory) were fed with high fat diet (HFD) starting at 6 weeks of age. At week eight, the mice were injected with either PBS or 2.5×10¹¹ genome copies (GC) of AAV8 vectors containing GG_F gene into hind limb both quadricep and gastrocnemius. Eight weeks old, male mice (Jackson Laboratory), which had been fed a low-fat diet (LFD), were injected with PBS as control group. Animal body weight was monitored before and after AAV injection weekly.

### Results:

To further determine the biological activity of the GLP-1 and GIP peptides expressed by the polycistronic expression constructs herein, HEK 293 cells were used that stably express the GLP-1 receptor or GIP receptor. The activity of GLP-1 and GIP peptide was assayed as described in the Experimental procedure.

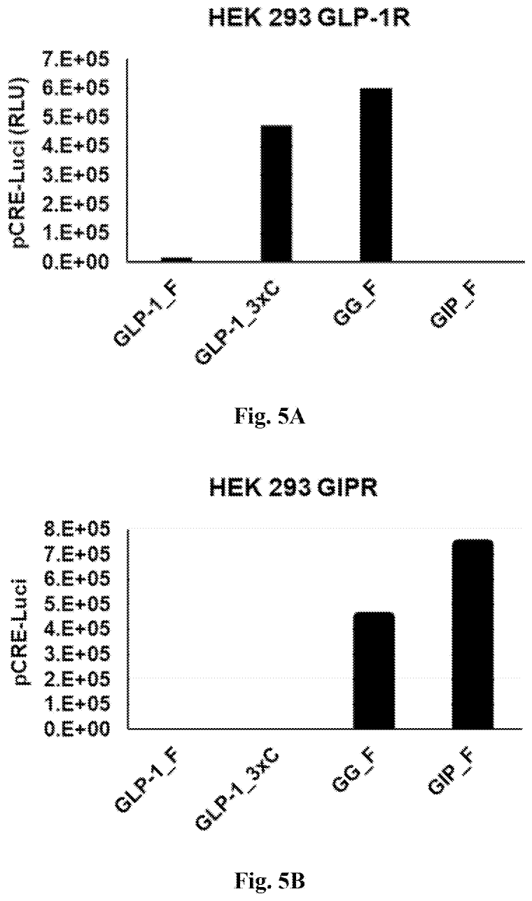

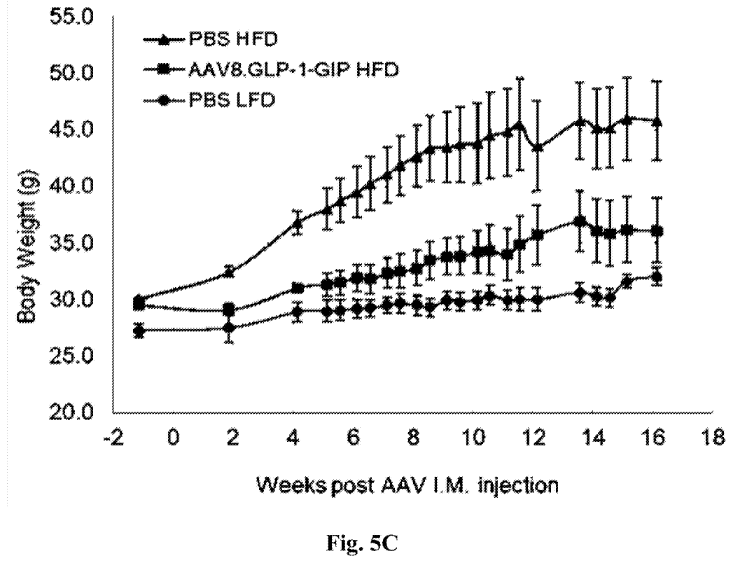

As shown in FIG. 5A, hGLP-1₇₋₃₇ peptide expressed from monocistronic expression construct GLP-1_F (expressing the hGLP-1₇₋₃₇ peptide, see Tables 5 and 6), bicistronic expression construct GG_F (expressing the hGLP-1₇₋₃₇ peptide and the hGIP₁₋₄₂ peptide, see Tables 7 and 8), and tricistronic expression construct GLP-1_3xC (expressing a polyprotein comprising three copies of the hGLP-1₇₋₃₇ peptide, see Tables 9 and 10) showed activities in activating CRE promoter-driven luciferase. Supernatant from a GIP construct-transfected culture that does not express the GLP-1 peptide did not show activity in HEK 293 cells expressing GLP-1 receptor (negative control).

As shown in FIG. 5B, hGIP₁₋₄₂ peptide expressed from bicistronic expression construct GG_F (expressing the hGLP-1₇₋₃₇ peptide and the hGIP₁₋₄₂ peptide, see Tables 7 and 8) and monocistronic expression construct GIP_F (expressing the hGIP₁₋₄₂ peptide, see Tables 5 and 6) activated CRE promoter driven luciferase gene. Supernatant from monocistronic expression construct GLP-1_F (expressing the hGLP-1₇₋₃₇ peptide, see Tables 5 and 6) or tricistronic expression construct GLP-1_3xC (expressing a polyprotein comprising three copies of the hGLP-17-37 peptide, see Tables 9 and 10) that does not express GIP did not activate CRE promoter driven luciferase gene (negative control). These results indicate that the gut peptides expressed from the polycistronic expression constructs disclosed herein are biologically active.

To further test the biological activities of the incretin peptides hGLP-1 and hGIP and their potential of inhibiting body weight gain from high fat diet (HFD), an AAV vector (comprising expression construct GG_F) was used to express the hGLP-1 and hGIP in mice fed with a high fat diet. Mice injected with AAV vector expressing hGLP-1 and hGIP gene had decreased body weight gain compared to mice without AAV.GG_F (FIG. 5C). This data indicates that hGLP-1 and hGIP expressed from expression constructs herein are biologically active and are useful in gene therapy for treating obesity.

### Example 6: Riboswitch Regulated Expression of GLP-1 and GIP Enhances Glucose Tolerance

### Experimental Procedure

Peptide encoding sequences were cloned into AAV plasmid backbone that contains AAV2 ITRs, CSAI promoter and human beta globin polyA sequence and packaged into AAV8 capsid, generating AAV8.GG_F_7-GLP-1 vectors (see Example 4).

For the animal study (FIG. 6A), Balb/c mice received intramuscular injection of total 2.5×10¹¹ viral genome (VG) per mouse of the receptive AAV8 viral particle into both quadriceps and both gastrocnemii. Compound 004 having the chemical structure

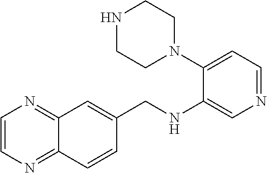 [SMILES: c1cc(N2CCNCC2)c(NCc2ccc3nccnc3c2)cn1]

was formulated in 0.5% methylcellulose (MC): 0.25% Tween® 80 in deionized (DI) water for oral administration. 30 days after AAV vector delivery, mice were treated orally via oral gavage with 300 mg/kg compound 004 for 4 days.

Nor the non-fasting intraperitoneal glucose tolerance test (IPGTT), mice (N=5) injected with AAV vectors were treated with 300 mg/kg compound 004 for 4 days and was given 300 mg/kg 5 hours before IPGTT. Blood glucose in tail vein blood was measured with hand-held glucose meter (CVS Health). The glucose was measured before glucose injection (0 min), then 15 min, 30 min, 60 min and 120 min after glucose (2 g/kg) was injected peritoneally.

### Results:

As shown in FIG. 6B, mice treated with the small molecule inducer showed better glucose tolerance than mice that received the dosing vehicle.

This shows that the GLP-1 and GIP peptides expressed by the injected vector increased the insulin release, thus improving glucose tolerance.

1. A bicistronic expression construct encoding a polyprotein, wherein:
(a) the polyprotein comprises a signal peptide, a first gut peptide, and a second gut peptide; and
(b) the polyprotein encoding sequence comprises:
(i) a sequence encoding the signal peptide;
(ii) a sequence encoding the first gut peptide;
(iii) a sequence encoding a first protease cleavage site, wherein the first protease cleavage site allows for the separation of the first gut peptide and the second gut peptide; and
(iv) a sequence encoding the second gut peptide.

2. A tricistronic expression construct encoding a polyprotein, wherein:
(a) the polyprotein comprises a signal peptide, a first gut peptide, a second gut peptide, and a third gut peptide; and
(b)_the polyprotein encoding sequence comprises:
(i) a sequence encoding the signal peptide;
(ii) a sequence encoding the first gut peptide;
(iii) a sequence encoding a first protease cleavage site, wherein the first protease cleavage site allows for the separation of the first gut peptide and the second gut peptide;
(iv) a sequence encoding the second gut peptide;
(v) a sequence encoding a second protease cleavage site allowing for the separation of the second gut peptide and the third gut peptide; and
(vi) a sequence encoding the third gut peptide.

3. The bicistronic expression construct of claim 1, wherein the signal peptide is selected from the group consisting of an immunoglobulin M (IgM) signal peptide, human insulin (hInsul) signal peptide, murine Igh protein (Igh) signal peptide, human growth hormone (hGH) signal peptide, murine erythropoietin (mEpo) signal peptide, murine growth hormone-releasing hormone (mGHRH) signal peptide, human albumin signal peptide, and human factor IX (FIX) signal peptide.

4. (canceled)

5. The bicistronic expression construct of claim 3, wherein (a) the signal peptide comprises a sequence that is at least 90% identical to any one of SEQ ID NOS:13-20 or (b) the sequence encoding the signal peptide comprises a sequence that is at least 90% identical to any one of SEQ ID NOS:21-28.

6. The bicistronic expression construct of claim 5, wherein (a) the signal peptide comprises any one of SEQ ID NOS:13-20 or (b) the sequence encoding the signal peptide comprises any one of SEQ ID NOS:21-28.

7.-9. (canceled)

10. The bicistronic expression construct of claim 1, wherein the first gut peptide and/or the second gut peptide encoded by the bicistronic expression construct independently comprises a sequence selected from the group consisting of human protein glp-1 (hGLP-1), human glucose dependent insulinotropic peptide (hGIP), human oxyntomodulin (hOXM), peptide YY (PYY), human glucagon, and amylin.

11. The bicistronic expression construct of claim 10, wherein (a) the first gut peptide and/or the second gut peptide independently comprises a sequence that is at least 90% identical to any one of SEQ ID NOS:1-5 or (b) the sequence encoding the first gut peptide and/or the second gut peptide independently comprises a sequence that is at least 90% identical to any one of SEQ ID NOS:6-12.

12. (canceled)

13. The bicistronic expression construct of claim 11, wherein (a) the first gut peptide gut peptide and/or the second gut peptide independently comprises any one of SEQ ID NOS:1-5 or the sequence encoding the first gut peptide and/or the second gut peptide independently comprises any one of SEQ ID NOS:6-12.

14.-25. (canceled)

26. The bicistronic expression construct of claim 1, wherein the sequence encoding the first gut peptide and/or the second gut peptide is codon-optimized.

27. (canceled)

28. The bicistronic expression construct of claim 1, wherein the first gut peptide and the second gut peptide are hGLP-1.

29. The bicistronic expression construct of claim 28, wherein (a) the first gut peptide and the second gut peptide each comprise a sequence that is at least 90% identical to SEQ ID NO:1 or (b) the sequences encoding the first and the second gut peptide each comprise a sequence that is at least 90% identical to any one of SEQ ID NOS:6-8.

30. (canceled)

31. The bicistronic expression construct of claim 29, wherein (a) the first gut peptide and the second gut peptide each comprise SEQ ID NO:1 or (b) the sequences encoding the first and the second gut peptide each comprise any one of SEQ ID NOS:6-8.

32.-34. (canceled)

35. The bicistronic expression construct of claim 28, wherein the bicistronic expression construct comprises (a) a sequence encoding a polypeptide that is at least 90% identical to SEQ ID NO:45 or SEQ ID NO:55 or (b) a sequence that is at least 90% identical to SEQ ID NO:50 or SEQ ID NO:57.

36. (canceled)

37. The bicistronic expression construct of claim 35, wherein the bicistronic expression construct comprises (a) a sequence encoding a polypeptide comprising SEQ ID NO:45 or SEQ ID NO:55 or (b) SEQ ID NO:50 or SEQ ID NO:57.

38.-41. (canceled)

42. The bicistronic expression construct of claim 1, wherein the first gut peptide and the second gut peptide are different and are independently selected from the group consisting of hGLP-1 and hGIP.

43. The bicistronic expression construct of claim 42, wherein the bicistronic expression construct comprises (a) a sequence encoding a polypeptide that is at least 90% identical to any one of SEQ ID NOS:46-49 or SEQ ID NO:56 or (b) a sequence that is at least 90% identical to any one of SEQ ID NOS:51-54 or SEQ ID NO:58.

44. (canceled)

45. The bicistronic expression construct of claim 43, wherein the bicistronic expression construct comprises (a) a sequence encoding a polypeptide comprising any one of SEQ ID NOS:46-49 or SEQ ID NO:56 or (b) any one of SEQ ID NOS:51-54 or SEQ ID NO:58.

46.-50. (canceled)

51. The tricistronic expression construct of claim 2, wherein the first gut peptide, the second gut peptide, and the third gut peptide are different and independently selected from the group consisting of:
(a) hGLP-1, hOXM, and PYY; and
(b) hGLP-1, hGlucagon, and hGIP.

52. The tricistronic expression construct of claim 51, wherein the tricistronic expression construct comprises (a) a sequence encoding a polypeptide that is at least 90% identical to any one of SEQ ID NOS:62-66 or SEQ ID NOS:76-77 or (b) comprises a sequence that is at least 90% identical to any one of SEQ ID NOS:70-74 or SEQ ID NOS:79-80.

53. (canceled)

54. The tricistronic expression construct of claim 52, wherein the tricistronic expression construct comprises (a) a sequence encoding a polypeptide comprising any one of SEQ ID NOS:62-66 or SEQ ID NOS:76-77 or (b) any one of SEQ ID NOS:70-74 or SEQ ID NOS:79-80.

55.-61. (canceled)

62. The tricistronic expression construct of claim 2, wherein the first gut peptide, the second gut peptide and the third gut peptide are hGLP-1.

63. The tricistronic expression construct of claim 62, wherein tricistronic expression construct comprises (a) a sequence encoding a polypeptide a sequence that is at least 90% identical to any one of SEQ ID NOS:59-61 or SEQ ID NO:75 or (b) a sequence that is at least 90% identical to any one of SEQ ID NOS:67-69 or SEQ ID NO:78.

64. (canceled)

65. The tricistronic expression construct of claim 6, wherein the tricistronic expression construct comprises (a) a sequence encoding a polypeptide comprising any one of SEQ ID NOS:59-61 or SEQ ID NO:75 or (b) any one of SEQ ID NOS:67-69 or SEQ ID NO:78.

66.-68. (canceled)

69. The bicistronic expression construct of claim 1, wherein the bicistronic expression construct further comprises a promoter sequence.

70. The bicistronic expression construct of claim 69, wherein the promoter is CMV promoter or a CASI promoter.

71. (canceled)

72. (canceled)

73. The bicistronic expression construct of claim 1, wherein the first protease cleavage site or the second protease cleavage site is a furin cleavage site.

74. The bicistronic expression construct of claim 1, wherein the polyprotein encoding sequence comprises a riboswitch comprising an aptamer, wherein the aptamer binds to a small molecule.

75. (canceled)

76. A vector comprising the bicistronic expression construct of claim 1.

77. The vector of claim 76, wherein the vector is an AAV vector.

78. A pharmaceutical composition comprising the vector of claim 76 and a pharmaceutically acceptable excipient.

79. A method of inducing satiation in a subject in need thereof, the method comprising administering to the subject the pharmaceutical composition of claim 78.

80. A method of treating obesity in a subject in need thereof, the method comprising administering to the subject the pharmaceutical composition of claim 78.

81. A method of suppressing appetite in a subject in need thereof, the method comprising administering to the subject the pharmaceutical composition of claim 78.

82. A method of reducing weight gain in a subject in need thereof, the method comprising administering to the subject the pharmaceutical composition of claim 78.

83. A method of improving glucose tolerance in a subject in need thereof, the method comprising administering to the subject the pharmaceutical composition of claim 78.

84. The bicistronic expression construct of claim 42, wherein (a) the first gut peptide comprises a sequence that is at least 90% identical to SEQ ID NO:1 and the second gut peptide comprises a sequence that is at least 90% identical to SEQ ID NO:2 or (b) the sequence encoding the first gut peptide comprises a sequence that is at least 90% identical to any one of SEQ ID NOs:6-8 and the sequence encoding the second gut peptide comprises a sequence that is at least 90% identical to SEQ ID NO:9.

85. The bicistronic expression construct of claim 84, wherein (a) the first gut peptide comprises SEQ ID NO:1 and the second gut peptide comprises SEQ ID NO:2 or (b) the sequence encoding the first gut peptide comprises any one of SEQ ID NOs:6-8 and the sequence encoding the second gut peptide comprises SEQ ID NO:9.

86. The tricistronic expression construct of claim 51, wherein:
(a) the first gut peptide comprises a sequence that is at least 90% identical to SEQ ID NO:1, the second gut peptide comprises a sequence that is at least 90% identical to SEQ ID NO:3, and the third gut peptide comprises a sequence that is at least 90% identical to SEQ ID NO:4; or
(b) the sequence encoding the first gut peptide comprises a sequence that is at least 90% identical to any one of SEQ ID NOs:6-8, the sequence encoding the second gut peptide comprises a sequence that is at least 90% identical to SEQ ID NO:10, and the sequence encoding the third gut peptide comprises a sequence that is at least 90% identical to SEQ ID NO:11.

87. The tricistronic expression construct of claim 86, wherein:
(a) the first gut peptide comprises SEQ ID NO:1, the second gut peptide comprises SEQ ID NO:3, and the third gut peptide comprises SEQ ID NO:4; or
(b) the sequence encoding the first gut peptide comprises any one of SEQ ID NOs:6-8, the sequence encoding the second gut peptide comprises SEQ ID NO:10, and the sequence encoding the third gut peptide comprises SEQ ID NO: 11.

88. The tricistronic expression construct of claim 51, wherein:
(a) the first gut peptide comprises a sequence that is at least 90% identical to SEQ ID NO:1, the second gut peptide comprises a sequence that is at least 90% identical to SEQ ID NO:5, and the third gut peptide comprises a sequence that is at least 90% identical to SEQ ID NO:2; or
(b) the sequence encoding the first gut peptide comprises a sequence that is at least 90% identical to any one of SEQ ID NOs:6-8, the sequence encoding the second gut peptide comprises a sequence that is at least 90% identical to SEQ ID NO:12, and the sequence encoding the third gut peptide comprises a sequence that is at least 90% identical to SEQ ID NO:9.

89. The tricistronic expression construct of claim 88, wherein:
(a) the first gut peptide comprises SEQ ID NO:1, the second gut peptide comprises SEQ ID NO:5, and the third gut peptide comprises SEQ ID NO:2; or
(b) the sequence encoding the first gut peptide comprises any one of SEQ ID NOs:6-8, the sequence encoding the second gut peptide comprises SEQ ID NO:12, and the sequence encoding the third gut peptide comprises SEQ ID NO:9.

90. The tricistronic expression construct of claim 52, wherein the tricistronic expression construct comprises (a) a sequence encoding a polypeptide that is at least 90% identical to SEQ ID NO:66 or (b) a sequence that is at least 90% identical to SEQ ID NO:77.

91. The tricistronic expression construct of claim 90, wherein the tricistronic expression construct comprises (a) a sequence encoding a polypeptide comprising SEQ ID NO:66 or (b) SEQ ID NO:77.

92. The tricistronic expression construct of claim 62, wherein (a) the first gut peptide, the second gut peptide, and the third gut peptide each comprise a sequence that is at least 90% identical to SEQ ID NO:1 or (b) the sequences encoding the first gut peptide, the second gut peptide, and the third gut peptide each comprise a sequence that is at least 90% identical to any one of SEQ ID NOS:6-8.

93. The tricistronic expression construct of claim 92, wherein (a) the first gut peptide, the second gut peptide, and the third gut peptide each comprise SEQ ID NO:1 or (b) the sequences encoding the first gut peptide, the second gut peptide, and the third gut peptide each comprise any one of SEQ ID NOS:6-8.

94. The method of claim 79, wherein the bicistronic expression construct further comprises a sequence encoding a third gut peptide, wherein the polyprotein further comprises a third gut peptide, and wherein the polyprotein further comprises a second protease cleavage site allowing for the separation of the second gut peptide and the third gut peptide.


SEQUENCE LISTING (117 sequences)

SEQ ID NO: 1 - protein, 31 aa, Homo sapiens
HAEGTFTSDVSSYLEGQAAKEFIAWLVKGRG

SEQ ID NO: 2 - protein, 42 aa, Homo sapiens
YAEGTFISDYSIAMDKIHQQDFVNWLLAQKGKKNDWKHNITQ

SEQ ID NO: 3 - protein, 37 aa, Homo sapiens
HSQGTFTSDYSKYLDSRRAQDFVQWLMNTKRNRNNIA

SEQ ID NO: 4 - protein, 34 aa, Homo sapiens
IKPEAPREDASPEELNRYYASLRHYLNLVTRQRY

SEQ ID NO: 5 - protein, 29 aa, Homo sapiens
HSQGTFTSDYSKYLDSRRAQDFVQWLMNT

SEQ ID NO: 6 - DNA, 93 bp, Homo sapiens
  source 1..93: mol_type = unassigned DNA
catgctgaagggacctttaccagtgatgtaagttcttatttggaaggccaagctgccaaggaattcattgcttggctggtgaaaggccgagga

SEQ ID NO: 7 - DNA, 93 bp, Homo sapiens
  source 1..93: mol_type = unassigned DNA
catgcagagggaacatttactagtgatgtcagttcatatcttgagggacaagctgctaaagaatttattgcttggcttgtgaagggaagagga

SEQ ID NO: 8 - DNA, 93 bp, Homo sapiens
  source 1..93: mol_type = unassigned DNA
catgctgaagggacatttacctcagatgtttcttcatacctggaaggacaggctgccaaggaatttattgcatggcttgtgaaaggcaggggc

SEQ ID NO: 9 - DNA, 126 bp, Homo sapiens
  source 1..126: mol_type = unassigned DNA
tacgcggaagggactttcatcagtgactacagtattgccatggacaagattcaccaacaagactttgtgaactggctgctggcccaaaaggggaagaagaatgactggaaacacaacatcacccag

SEQ ID NO: 10 - DNA, 111 bp, Homo sapiens
  source 1..111: mol_type = unassigned DNA
cattcacagggcacattcaccagtgactacagcaagtatctggactccaggcgtgcccaagattttgtgcagtggttgatgaataccaagaggaacaggaataacattgcc

SEQ ID NO: 11 - DNA, 102 bp, Homo sapiens
  source 1..102: mol_type = unassigned DNA
atcaaacccgaggctccccgcgaagacgcctcgccggaggagctgaaccgctactacgcctccctgcgccactacctcaacctggtcacccggcagcggtat

SEQ ID NO: 12 - DNA, 87 bp, Homo sapiens
  source 1..87: mol_type = unassigned DNA
cattcacagggcacattcaccagtgactacagcaagtatctggactccaggcgtgcccaagattttgtgcagtggttgatgaatacc

SEQ ID NO: 13 - protein, 20 aa, Homo sapiens
MGWSCIILFLVATATGAHSA

SEQ ID NO: 14 - protein, 24 aa, Homo sapiens
MALWMRLLPLLALLALWGPDPAAA

SEQ ID NO: 15 - protein, 19 aa, Mus musculus
MAWVWTLLFLMAAAQSIQA

SEQ ID NO: 16 - protein, 26 aa, Homo sapiens
MATGSRTSLLLAFGLLCLPWLQEGSA

SEQ ID NO: 17 - protein, 26 aa, Mus musculus
MGVPERPTLLLLLSLLLIPLGLPVLC

SEQ ID NO: 18 - protein, 19 aa, Mus musculus
MLLWVLFVILILTSGSHCS

SEQ ID NO: 19 - protein, 18 aa, Homo sapiens
MKWVTFISLLFLFSSAYS

SEQ ID NO: 20 - protein, 28 aa, Homo sapiens
MQRVNMIMAESPGLITICLLGYLLSAEC

SEQ ID NO: 21 - DNA, 60 bp, Homo sapiens
  source 1..60: mol_type = unassigned DNA
atgggatggagctgtatcatcctcttcttggtagcaacagctacaggcgcgcacagtgca

SEQ ID NO: 22 - DNA, 72 bp, Homo sapiens
  source 1..72: mol_type = unassigned DNA
atggccctgtggatgcgcctcctgcccctgctggcgctgctggccctctggggacctgacccagccgcagcc

SEQ ID NO: 23 - DNA, 57 bp, Mus musculus
  source 1..57: mol_type = unassigned DNA
atggcctgggtctggacactcctgtttctgatggctgctgcccagtccattcaggcc

SEQ ID NO: 24 - DNA, 78 bp, Homo sapiens
  source 1..78: mol_type = unassigned DNA
atggctacaggctcccggacgtccctgctcctggcttttggcctgctctgcctgccctggcttcaagagggcagtgcc

SEQ ID NO: 25 - DNA, 78 bp, Mus musculus
  source 1..78: mol_type = unassigned DNA
atgggggtgcccgaacgtcccaccctgctgcttttactctccttgctactgattcctctgggcctcccagtcctctgt

SEQ ID NO: 26 - DNA, 57 bp, Mus musculus
  source 1..57: mol_type = unassigned DNA
atgctgctctgggtgctctttgtgatcctcatcctcaccagtggctcccactgctca

SEQ ID NO: 27 - DNA, 54 bp, Homo sapiens
  source 1..54: mol_type = unassigned DNA
atgaagtgggtaacctttatttcccttctttttctctttagctcggcttattcc

SEQ ID NO: 28 - DNA, 84 bp, Homo sapiens
  source 1..84: mol_type = unassigned DNA
atgcagcgcgtgaacatgatcatggcagaatcaccaggcctcatcaccatctgccttttaggatatctactcagtgctgaatgt

SEQ ID NO: 29 - protein, 64 aa, synthetic construct
MGWSCIILFLVATATGAHSAYPYDVPDYARKKRHAEGTFTSDVSSYLEGQAAKEFIAWLVKGRG

SEQ ID NO: 30 - protein, 68 aa, synthetic construct
MALWMRLLPLLALLALWGPDPAAAYPYDVPDYARKKRHAEGTFTSDVSSYLEGQAAKEFIAWLVKGRG

SEQ ID NO: 31 - protein, 63 aa, synthetic construct
MAWVWTLLFLMAAAQSIQAYPYDVPDYARKKRHAEGTFTSDVSSYLEGQAAKEFIAWLVKGRG

SEQ ID NO: 32 - protein, 78 aa, synthetic construct
MATGSRTSLLLAFGLLCLPWLQEGSAFPTIPLSRLFDNAMLRARKKRHAEGTFTSDVSSYLEGQAAKEFIAWLVKGRG

SEQ ID NO: 33 - protein, 76 aa, synthetic construct
MGVPERPTLLLLLSLLLIPLGLPVLCAPPRLICDSRVLERYRKKRHAEGTFTSDVSSYLEGQAAKEFIAWLVKGRG

SEQ ID NO: 34 - protein, 61 aa, synthetic construct
MLLWVLFVILILTSGSHCSLPPSPPFRMQRHAEGTFTSDVSSYLEGQAAKEFIAWLVKGRG

SEQ ID NO: 35 - protein, 55 aa, synthetic construct
MKWVTFISLLFLFSSAYSRGVFRRHAEGTFTSDVSSYLEGQAAKEFIAWLVKGRG

SEQ ID NO: 36 - protein, 77 aa, synthetic construct
MQRVNMIMAESPGLITICLLGYLLSAECTVFLDHENANKILNRPKRHAEGTFTSDVSSYLEGQAAKEFIAWLVKGRG

SEQ ID NO: 37 - DNA, 195 bp, synthetic construct
  source 1..195: mol_type = other DNA
atgggatggagctgtatcatcctcttcttggtagcaacagctacaggcgcgcacagtgcatacccatacgatgttccagattacgctagaaaaaagagacatgctgaagggacctttaccagtgatgtaagttcttatttggaaggccaagctgccaaggaattcattgcttggctggtgaaaggccgaggatga

SEQ ID NO: 38 - DNA, 207 bp, synthetic construct
  source 1..207: mol_type = other DNA
atggccctgtggatgcgcctcctgcccctgctggcgctgctggccctctggggacctgacccagccgcagcctacccatacgatgttccagattacgctagaaaaaagagacatgctgaagggacctttaccagtgatgtaagttcttatttggaaggccaagctgccaaggaattcattgcttggctggtgaaaggccgaggatga

SEQ ID NO: 39 - DNA, 192 bp, synthetic construct
  source 1..192: mol_type = other DNA
atggcctgggtctggacactcctgtttctgatggctgctgcccagtccattcaggcctacccatacgatgttccagattacgctagaaaaaagagacatgctgaagggacctttaccagtgatgtaagttcttatttggaaggccaagctgccaaggaattcattgcttggctggtgaaaggccgaggatga

SEQ ID NO: 40 - DNA, 237 bp, synthetic construct
  source 1..237: mol_type = other DNA
atggctacaggctcccggacgtccctgctcctggcttttggcctgctctgcctgccctggcttcaagagggcagtgccttcccaaccattcccttatccaggctttttgacaacgctatgctccgcgccagaaaaaagagacatgctgaagggacctttaccagtgatgtaagttcttatttggaaggccaagctgccaaggaattcattgcttggctggtgaaaggccgaggatga

SEQ ID NO: 41 - DNA, 231 bp, synthetic construct
  source 1..231: mol_type = other DNA
atgggggtgcccgaacgtcccaccctgctgcttttactctccttgctactgattcctctgggcctcccagtcctctgtgctcccccacgcctcatctgcgacagtcgagttctggagaggtacagaaaaaagagacatgctgaagggacctttaccagtgatgtaagttcttatttggaaggccaagctgccaaggaattcattgcttggctggtgaaaggccgaggatga

SEQ ID NO: 42 - DNA, 186 bp, synthetic construct
  source 1..186: mol_type = other DNA
atgctgctctgggtgctctttgtgatcctcatcctcaccagtggctcccactgctcactgcccccctcacctcccttcaggatgcagcgacatgctgaagggacctttaccagtgatgtaagttcttatttggaaggccaagctgccaaggaattcattgcttggctggtgaaaggccgaggatga

SEQ ID NO: 43 - DNA, 168 bp, synthetic construct
  source 1..168: mol_type = other DNA
atgaagtgggtaacctttatttcccttctttttctctttagctcggcttattccaggggtgtgtttcgtcgacatgctgaagggacctttaccagtgatgtaagttcttatttggaaggccaagctgccaaggaattcattgcttggctggtgaaaggccgaggatga

SEQ ID NO: 44 - DNA, 234 bp, synthetic construct
  source 1..234: mol_type = other DNA
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggcatgctgaagggacctttaccagtgatgtaagttcttatttggaaggccaagctgccaaggaattcattgcttggctggtgaaaggccgaggatga

SEQ ID NO: 45 - protein, 90 aa, synthetic construct
MKWVTFISLLFLFSSAYSRGVFRRHAEGTFTSDVSSYLEGQAAKEFIAWLVKGRGRKKRHAEGTFTSDVSSYLEGQAAKEFIAWLVKGRG

SEQ ID NO: 46 - protein, 124 aa, synthetic construct
MATGSRTSLLLAFGLLCLPWLQEGSAFPTIPLSRLFDNAMLRARKKRHAEGTFTSDVSSYLEGQAAKEFIAWLVKGRGRKKRYAEGTFISDYSIAMDKIHQQDFVNWLLAQKGKKNDWKHNITQ

SEQ ID NO: 47 - protein, 114 aa, synthetic construct
MALWMRLLPLLALLALWGPDPAAAYPYDVPDYARKKRHAEGTFTSDVSSYLEGQAAKEFIAWLVKGRGRKKRYAEGTFISDYSIAMDKIHQQDFVNWLLAQKGKKNDWKHNITQ

SEQ ID NO: 48 - protein, 107 aa, synthetic construct
MLLWVLFVILILTSGSHCSLPPSPPFRMQRHAEGTFTSDVSSYLEGQAAKEFIAWLVKGRGRKKRYAEGTFISDYSIAMDKIHQQDFVNWLLAQKGKKNDWKHNITQ

SEQ ID NO: 49 - protein, 101 aa, synthetic construct
MKWVTFISLLFLFSSAYSRGVFRRHAEGTFTSDVSSYLEGQAAKEFIAWLVKGRGRKKRYAEGTFISDYSIAMDKIHQQDFVNWLLAQKGKKNDWKHNITQ

SEQ ID NO: 50 - DNA, 273 bp, synthetic construct
  source 1..273: mol_type = other DNA
atgaagtgggtaacctttatttcccttctttttctctttagctcggcttattccaggggtgtgtttcgtcgacatgctgaagggacctttaccagtgatgtaagttcttatttggaaggccaagctgccaaggaattcattgcttggctggtgaaaggccgaggaagaaaaaagagacatgcagagggaacatttactagtgatgtcagttcatatcttgagggacaagctgctaaagaatttattgcttggcttgtgaagggaagaggatga

SEQ ID NO: 51 - DNA, 375 bp, synthetic construct
  source 1..375: mol_type = other DNA
atggctacaggctcccggacgtccctgctcctggcttttggcctgctctgcctgccctggcttcaagagggcagtgccttcccaaccattcccttatccaggctttttgacaacgctatgctccgcgccagaaaaaagagacatgctgaagggacctttaccagtgatgtaagttcttatttggaaggccaagctgccaaggaattcattgcttggctggtgaaaggccgaggaagaaaaaagagatacgcggaagggactttcatcagtgactacagtattgccatggacaagattcaccaacaagactttgtgaactggctgctggcccaaaaggggaagaagaatgactggaaacacaacatcacccagtga

SEQ ID NO: 52 - DNA, 345 bp, synthetic construct
  source 1..345: mol_type = other DNA
atggccctgtggatgcgcctcctgcccctgctggcgctgctggccctctggggacctgacccagccgcagcctacccatacgatgttccagattacgctagaaaaaagagacatgctgaagggacctttaccagtgatgtaagttcttatttggaaggccaagctgccaaggaattcattgcttggctggtgaaaggccgaggaagaaaaaagagatacgcggaagggactttcatcagtgactacagtattgccatggacaagattcaccaacaagactttgtgaactggctgctggcccaaaaggggaagaagaatgactggaaacacaacatcacccagtga

SEQ ID NO: 53 - DNA, 324 bp, synthetic construct
  source 1..324: mol_type = other DNA
atgctgctctgggtgctctttgtgatcctcatcctcaccagtggctcccactgctcactgcccccctcacctcccttcaggatgcagcgacatgctgaagggacctttaccagtgatgtaagttcttatttggaaggccaagctgccaaggaattcattgcttggctggtgaaaggccgaggaagaaaaaagagatacgcggaagggactttcatcagtgactacagtattgccatggacaagattcaccaacaagactttgtgaactggctgctggcccaaaaggggaagaagaatgactggaaacacaacatcacccagtga

SEQ ID NO: 54 - DNA, 306 bp, synthetic construct
  source 1..306: mol_type = other DNA
atgaagtgggtaacctttatttcccttctttttctctttagctcggcttattccaggggtgtgtttcgtcgacatgctgaagggacctttaccagtgatgtaagttcttatttggaaggccaagctgccaaggaattcattgcttggctggtgaaaggccgaggaagaaaaaagagatacgcggaagggactttcatcagtgactacagtattgccatggacaagattcaccaacaagactttgtgaactggctgctggcccaaaaggggaagaagaatgactggaaacacaacatcacccagtga

SEQ ID NO: 55 - protein, 66 aa, synthetic construct
HAEGTFTSDVSSYLEGQAAKEFIAWLVKGRGRKKRHAEGTFTSDVSSYLEGQAAKEFIAWLVKGRG

SEQ ID NO: 56 - protein, 77 aa, synthetic construct
HAEGTFTSDVSSYLEGQAAKEFIAWLVKGRGRKKRYAEGTFISDYSIAMDKIHQQDFVNWLLAQKGKKNDWKHNITQ

SEQ ID NO: 57 - DNA, 201 bp, synthetic construct
  source 1..201: mol_type = other DNA
catgctgaagggacctttaccagtgatgtaagttcttatttggaaggccaagctgccaaggaattcattgcttggctggtgaaaggccgaggaagaaaaaagagacatgcagagggaacatttactagtgatgtcagttcatatcttgagggacaagctgctaaagaatttattgcttggcttgtgaagggaagaggatga

SEQ ID NO: 58 - DNA, 234 bp, synthetic construct
  source 1..234: mol_type = other DNA
catgctgaagggacctttaccagtgatgtaagttcttatttggaaggccaagctgccaaggaattcattgcttggctggtgaaaggccgaggaagaaaaaagagatacgcggaagggactttcatcagtgactacagtattgccatggacaagattcaccaacaagactttgtgaactggctgctggcccaaaaggggaagaagaatgactggaaacacaacatcacccagtga

SEQ ID NO: 59 - protein, 125 aa, synthetic construct
MKWVTFISLLFLFSSAYSRGVFRRHAEGTFTSDVSSYLEGQAAKEFIAWLVKGRGRKKRHAEGTFTSDVSSYLEGQAAKEFIAWLVKGRGRKKRHAEGTFTSDVSSYLEGQAAKEFIAWLVKGRG

SEQ ID NO: 60 - protein, 138 aa, synthetic construct
MALWMRLLPLLALLALWGPDPAAAYPYDVPDYARKKRHAEGTFTSDVSSYLEGQAAKEFIAWLVKGRGRKKRHAEGTFTSDVSSYLEGQAAKEFIAWLVKGRGRKKRHAEGTFTSDVSSYLEGQAAKEFIAWLVKGRG

SEQ ID NO: 61 - protein, 131 aa, synthetic construct
MLLWVLFVILILTSGSHCSLPPSPPFRMQRHAEGTFTSDVSSYLEGQAAKEFIAWLVKGRGRKKRHAEGTFTSDVSSYLEGQAAKEFIAWLVKGRGRKKRHAEGTFTSDVSSYLEGQAAKEFIAWLVKGRG

SEQ ID NO: 62 - protein, 157 aa, synthetic construct
MATGSRTSLLLAFGLLCLPWLQEGSAFPTIPLSRLFDNAMLRARKKRHAEGTFTSDVSSYLEGQAAKEFIAWLVKGRGRRKRHSQGTFTSDYSKYLDSRRAQDFVQWLMNTKRNRNNIARKKRIKPEAPREDASPEELNRYYASLRHYLNLVTRQRY

SEQ ID NO: 63 - protein, 147 aa, synthetic construct
MALWMRLLPLLALLALWGPDPAAAYPYDVPDYARKKRHAEGTFTSDVSSYLEGQAAKEFIAWLVKGRGRRKRHSQGTFTSDYSKYLDSRRAQDFVQWLMNTKRNRNNIARKKRIKPEAPREDASPEELNRYYASLRHYLNLVTRQRY

SEQ ID NO: 64 - protein, 140 aa, synthetic construct
MLLWVLFVILILTSGSHCSLPPSPPFRMQRHAEGTFTSDVSSYLEGQAAKEFIAWLVKGRGRRKRHSQGTFTSDYSKYLDSRRAQDFVQWLMNTKRNRNNIARKKRIKPEAPREDASPEELNRYYASLRHYLNLVTRQRY

SEQ ID NO: 65 - protein, 134 aa, synthetic construct
MKWVTFISLLFLFSSAYSRGVFRRHAEGTFTSDVSSYLEGQAAKEFIAWLVKGRGRRKRHSQGTFTSDYSKYLDSRRAQDFVQWLMNTKRNRNNIARKKRIKPEAPREDASPEELNRYYASLRHYLNLVTRQRY

SEQ ID NO: 66 - protein, 157 aa, synthetic construct
MATGSRTSLLLAFGLLCLPWLQEGSAFPTIPLSRLFDNAMLRARKKRHAEGTFTSDVSSYLEGQAAKEFIAWLVKGRGRRKRHSQGTFTSDYSKYLDSRRAQDFVQWLMNTRKKRYAEGTFISDYSIAMDKIHQQDFVNWLLAQKGKKNDWKHNITQ

SEQ ID NO: 67 - DNA, 378 bp, synthetic construct
  source 1..378: mol_type = other DNA
atgaagtgggtaacctttatttcccttctttttctctttagctcggcttattccaggggtgtgtttcgtcgacatgctgaagggacctttaccagtgatgtaagttcttatttggaaggccaagctgccaaggaattcattgcttggctggtgaaaggccgaggaagaaaaaagagacatgcagagggaacatttactagtgatgtcagttcatatcttgagggacaagctgctaaagaatttattgcttggcttgtgaagggaagaggaagaaagaagagacatgctgaagggacatttacctcagatgtttcttcatacctggaaggacaggctgccaaggaatttattgcatggcttgtgaaaggcaggggctga

SEQ ID NO: 68 - DNA, 417 bp, synthetic construct
  source 1..417: mol_type = other DNA
atggccctgtggatgcgcctcctgcccctgctggcgctgctggccctctggggacctgacccagccgcagcctacccatacgatgttccagattacgctagaaaaaagagacatgctgaagggacctttaccagtgatgtaagttcttatttggaaggccaagctgccaaggaattcattgcttggctggtgaaaggccgaggaagaaaaaagagacatgcagagggaacatttactagtgatgtcagttcatatcttgagggacaagctgctaaagaatttattgcttggcttgtgaagggaagaggaagaaagaagagacatgctgaagggacatttacctcagatgtttcttcatacctggaaggacaggctgccaaggaatttattgcatggcttgtgaaaggcaggggctga

SEQ ID NO: 69 - DNA, 396 bp, synthetic construct
  source 1..396: mol_type = other DNA
atgctgctctgggtgctctttgtgatcctcatcctcaccagtggctcccactgctcactgcccccctcacctcccttcaggatgcagcgacatgctgaagggacctttaccagtgatgtaagttcttatttggaaggccaagctgccaaggaattcattgcttggctggtgaaaggccgaggaagaaaaaagagacatgcagagggaacatttactagtgatgtcagttcatatcttgagggacaagctgctaaagaatttattgcttggcttgtgaagggaagaggaagaaagaagagacatgctgaagggacatttacctcagatgtttcttcatacctggaaggacaggctgccaaggaatttattgcatggcttgtgaaaggcaggggctga

SEQ ID NO: 70 - DNA, 474 bp, synthetic construct
  source 1..474: mol_type = other DNA
atggctacaggctcccggacgtccctgctcctggcttttggcctgctctgcctgccctggcttcaagagggcagtgccttcccaaccattcccttatccaggctttttgacaacgctatgctccgcgccagaaaaaagagacatgctgaagggacctttaccagtgatgtaagttcttatttggaaggccaagctgccaaggaattcattgcttggctggtgaaaggccgaggaagacgtaagaggcattcacagggcacattcaccagtgactacagcaagtatctggactccaggcgtgcccaagattttgtgcagtggttgatgaataccaagaggaacaggaataacattgccagaaaaaagagaatcaaacccgaggctccccgcgaagacgcctcgccggaggagctgaaccgctactacgcctccctgcgccactacctcaacctggtcacccggcagcggtattga

SEQ ID NO: 71 - DNA, 444 bp, synthetic construct
  source 1..444: mol_type = other DNA
atggccctgtggatgcgcctcctgcccctgctggcgctgctggccctctggggacctgacccagccgcagcctacccatacgatgttccagattacgctagaaaaaagagacatgctgaagggacctttaccagtgatgtaagttcttatttggaaggccaagctgccaaggaattcattgcttggctggtgaaaggccgaggaagacgtaagaggcattcacagggcacattcaccagtgactacagcaagtatctggactccaggcgtgcccaagattttgtgcagtggttgatgaataccaagaggaacaggaataacattgccagaaaaaagagaatcaaacccgaggctccccgcgaagacgcctcgccggaggagctgaaccgctactacgcctccctgcgccactacctcaacctggtcacccggcagcggtattga

SEQ ID NO: 72 - DNA, 423 bp, synthetic construct
  source 1..423: mol_type = other DNA
atgctgctctgggtgctctttgtgatcctcatcctcaccagtggctcccactgctcactgcccccctcacctcccttcaggatgcagcgacatgctgaagggacctttaccagtgatgtaagttcttatttggaaggccaagctgccaaggaattcattgcttggctggtgaaaggccgaggaagacgtaagaggcattcacagggcacattcaccagtgactacagcaagtatctggactccaggcgtgcccaagattttgtgcagtggttgatgaataccaagaggaacaggaataacattgccagaaaaaagagaatcaaacccgaggctccccgcgaagacgcctcgccggaggagctgaaccgctactacgcctccctgcgccactacctcaacctggtcacccggcagcggtattga

SEQ ID NO: 73 - DNA, 405 bp, synthetic construct
  source 1..405: mol_type = other DNA
atgaagtgggtaacctttatttcccttctttttctctttagctcggcttattccaggggtgtgtttcgtcgacatgctgaagggacctttaccagtgatgtaagttcttatttggaaggccaagctgccaaggaattcattgcttggctggtgaaaggccgaggaagacgtaagaggcattcacagggcacattcaccagtgactacagcaagtatctggactccaggcgtgcccaagattttgtgcagtggttgatgaataccaagaggaacaggaataacattgccagaaaaaagagaatcaaacccgaggctccccgcgaagacgcctcgccggaggagctgaaccgctactacgcctccctgcgccactacctcaacctggtcacccggcagcggtattga

SEQ ID NO: 74 - DNA, 474 bp, synthetic construct
  source 1..474: mol_type = other DNA
atggctacaggctcccggacgtccctgctcctggcttttggcctgctctgcctgccctggcttcaagagggcagtgccttcccaaccattcccttatccaggctttttgacaacgctatgctccgcgccagaaaaaagagacatgctgaagggacctttaccagtgatgtaagttcttatttggaaggccaagctgccaaggaattcattgcttggctggtgaaaggccgaggaagacgtaagaggcattcacagggcacattcaccagtgactacagcaagtatctggactccaggcgtgcccaagattttgtgcagtggttgatgaataccagaaaaaagagatacgcggaagggactttcatcagtgactacagtattgccatggacaagattcaccaacaagactttgtgaactggctgctggcccaaaaggggaagaagaatgactggaaacacaacatcacccagtga

SEQ ID NO: 75 - protein, 101 aa, synthetic construct
HAEGTFTSDVSSYLEGQAAKEFIAWLVKGRGRKKRHAEGTFTSDVSSYLEGQAAKEFIAWLVKGRGRKKRHAEGTFTSDVSSYLEGQAAKEFIAWLVKGRG

SEQ ID NO: 76 - protein, 110 aa, synthetic construct
HAEGTFTSDVSSYLEGQAAKEFIAWLVKGRGRRKRHSQGTFTSDYSKYLDSRRAQDFVQWLMNTKRNRNNIARKKRIKPEAPREDASPEELNRYYASLRHYLNLVTRQRY

SEQ ID NO: 77 - protein, 110 aa, synthetic construct
HAEGTFTSDVSSYLEGQAAKEFIAWLVKGRGRRKRHSQGTFTSDYSKYLDSRRAQDFVQWLMNTRKKRYAEGTFISDYSIAMDKIHQQDFVNWLLAQKGKKNDWKHNITQ

SEQ ID NO: 78 - DNA, 306 bp, synthetic construct
  source 1..306: mol_type = other DNA
catgctgaagggacctttaccagtgatgtaagttcttatttggaaggccaagctgccaaggaattcattgcttggctggtgaaaggccgaggaagaaaaaagagacatgcagagggaacatttactagtgatgtcagttcatatcttgagggacaagctgctaaagaatttattgcttggcttgtgaagggaagaggaagaaagaagagacatgctgaagggacatttacctcagatgtttcttcatacctggaaggacaggctgccaaggaatttattgcatggcttgtgaaaggcaggggctga

SEQ ID NO: 79 - DNA, 333 bp, synthetic construct
  source 1..333: mol_type = other DNA
catgctgaagggacctttaccagtgatgtaagttcttatttggaaggccaagctgccaaggaattcattgcttggctggtgaaaggccgaggaagacgtaagaggcattcacagggcacattcaccagtgactacagcaagtatctggactccaggcgtgcccaagattttgtgcagtggttgatgaataccaagaggaacaggaataacattgccagaaaaaagagaatcaaacccgaggctccccgcgaagacgcctcgccggaggagctgaaccgctactacgcctccctgcgccactacctcaacctggtcacccggcagcggtattga

SEQ ID NO: 80 - DNA, 333 bp, synthetic construct
  source 1..333: mol_type = other DNA
catgctgaagggacctttaccagtgatgtaagttcttatttggaaggccaagctgccaaggaattcattgcttggctggtgaaaggccgaggaagacgtaagaggcattcacagggcacattcaccagtgactacagcaagtatctggactccaggcgtgcccaagattttgtgcagtggttgatgaataccagaaaaaagagatacgcggaagggactttcatcagtgactacagtattgccatggacaagattcaccaacaagactttgtgaactggctgctggcccaaaaggggaagaagaatgactggaaacacaacatcacccagtga

SEQ ID NO: 81 - DNA, 39 bp, synthetic construct
  source 1..39: mol_type = other DNA
tacccatacgatgttccagattacgctagaaaaaagaga

SEQ ID NO: 82-83 - skipped (2 entries)

SEQ ID NO: 84 - DNA, 63 bp, synthetic construct
  source 1..63: mol_type = other DNA
ttcccaaccattcccttatccaggctttttgacaacgctatgctccgcgccagaaaaaagaga

SEQ ID NO: 85 - DNA, 57 bp, synthetic construct
  source 1..57: mol_type = other DNA
gctcccccacgcctcatctgcgacagtcgagttctggagaggtacagaaaaaagaga

SEQ ID NO: 86 - DNA, 33 bp, synthetic construct
  source 1..33: mol_type = other DNA
ctgcccccctcacctcccttcaggatgcagcga

SEQ ID NO: 87 - DNA, 18 bp, synthetic construct
  source 1..18: mol_type = other DNA
aggggtgtgtttcgtcga

SEQ ID NO: 88 - DNA, 54 bp, synthetic construct
  source 1..54: mol_type = other DNA
acagtttttcttgatcatgaaaacgccaacaaaattctgaatcggccaaagagg

SEQ ID NO: 89 - protein, 13 aa, synthetic construct
YPYDVPDYARKKR

SEQ ID NO: 90-91 - skipped (2 entries)

SEQ ID NO: 92 - protein, 21 aa, synthetic construct
FPTIPLSRLFDNAMLRARKKR

SEQ ID NO: 93 - protein, 19 aa, synthetic construct
APPRLICDSRVLERYRKKR

SEQ ID NO: 94 - protein, 11 aa, synthetic construct
LPPSPPFRMQR

SEQ ID NO: 95 - protein, 6 aa, synthetic construct
RGVFRR

SEQ ID NO: 96 - protein, 18 aa, synthetic construct
TVFLDHENANKILNRPKR

SEQ ID NO: 97 - protein, 4 aa, synthetic construct
RKKR

SEQ ID NO: 98 - protein, 4 aa, synthetic construct
RMQR

SEQ ID NO: 99 - protein, 4 aa, synthetic construct
VFRR

SEQ ID NO: 100 - protein, 4 aa, synthetic construct
RPKR

SEQ ID NO: 101 - DNA, 316 bp, synthetic construct
  source 1..316: mol_type = other DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgccgagtaacgctgtttctctaacttgtaggaatgaattcagatatttccagagaatgaaaaaaaaatcttcagtagaaggtaatgt

SEQ ID NO: 102 - skipped

SEQ ID NO: 103 - DNA, 50 bp, synthetic construct
  source 1..50: mol_type = other DNA
gaatgaattcagatatttccagagaatgaaaaaaaaatcttcagtagaag

SEQ ID NO: 104 - DNA, 49 bp, synthetic construct
  source 1..49: mol_type = other DNA
gaatgaattcagatatttccagagaatgaaaaaaaatcttcagtagaag

SEQ ID NO: 105-106 - skipped (2 entries)

SEQ ID NO: 107 - DNA, 10 bp, synthetic construct
  source 1..10: mol_type = other DNA
caggtaagta

SEQ ID NO: 108 - RNA, 10 nt, synthetic construct
  source 1..10: mol_type = other RNA
caggtaagta

SEQ ID NO: 109-110 - skipped (2 entries)

SEQ ID NO: 111 - protein, 75 aa, synthetic construct
MGWSCIILFLVATATGAHSAYPYDVPDYARKKRYAEGTFISDYSIAMDKIHQQDFVNWLLAQKGKKNDWKHNITQ

SEQ ID NO: 112 - protein, 88 aa, synthetic construct
MQRVNMIMAESPGLITICLLGYLLSAECTVFLDHENANKILNRPKRYAEGTFISDYSIAMDKIHQQDFVNWLLAQKGKKNDWKHNITQ

SEQ ID NO: 113 - protein, 66 aa, synthetic construct
MKWVTFISLLFLFSSAYSRGVFRRYAEGTFISDYSIAMDKIHQQDFVNWLLAQKGKKNDWKHNITQ

SEQ ID NO: 114 - protein, 89 aa, synthetic construct
MATGSRTSLLLAFGLLCLPWLQEGSAFPTIPLSRLFDNAMLRARKKRYAEGTFISDYSIAMDKIHQQDFVNWLLAQKGKKNDWKHNITQ

SEQ ID NO: 115 - DNA, 108 bp, synthetic construct
  source 1..108: mol_type = other DNA
agaaagaagagacatgctgaagggacatttacctcagatgtttcttcatacctggaaggacaggctgccaaggaatttattgcatggcttgtgaaaggcaggggctga

SEQ ID NO: 116 - DNA, 246 bp, synthetic construct
  source 1..246: mol_type = other DNA
acattacgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 117 - DNA, 651 bp, synthetic construct
  source 1..651: mol_type = other DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgccgagtaacgctgtttctctaacttgtaggaatgaattcagatatttccagagaatgaaaaaaaaatcttcagtagaaggtaatgtctggggagtccttcatgcggggctgagaggatggaagcaatcgaccatcgacccattgcacctgatccggtatgtcccggcgcagggagacattacgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag